SEQUENCE LISTING (65 sequences)

SEQ ID NO: 1 - DNA, 1398 bp, Agrobacterium tumefaciens
attaaaagcatcgggaaaggaaggaaaatttatggctttttctaatgctgccccgattgctaacagacggtggtctgaccccgctaatccatcaatttccattccaacaggcaagcgatcaggtgtaaggcaggcaggaaggctcaacccaggtaggcctgcgttgctgcttgggtccacatttcgcacgtagatcttgaaagtgttcatcattgagccattgtggatgactgacgactcctgacctatggctttggccgctaagggtgcagttgggaaaaggattgcatctaactgatagagtctgaagtaattccgataagtggcctggagccttggcctgaaggattgacgcgccagttcatattcatcgttggaaatttgatgcccatcaatttgcgcactgacaatgttcgctacatcggggctacgaattctttgaaacgtcagaaaaagaaatgttcccacaaaatcgtccagatacttttttagagcgtgtggaaattcgtaaagcgcaattggcaaacttgccccactattcagttcctctaggtgggggatgtcggcttcaacaaaggttacgcctctgttggctagcaagcgaatcgtcgtttcagctgcgaaggccacatcagcatcaaggtcatcgtaaaagtaggtagtggggaggccgatccgaagccccttcagcggcatgggtaaaattttcgccgaccgtccggaaatcacctggtcgaggattataacatcggctacgcactgcgctatgattccggcggtgtcccgggtggggctgaccggtattatccgatctcttggatatcgagcaagcgtcggtcgaaatcctactacgccacacagggctgcgggtaggcgaacagatgcaccggtatcggtgcctatgccgcctaacatcaatcggcttgccaccgcagcagccacaccaccgcttgaacctcctggtatcaaacttggattccacgggttccgcaccgcaccggtggcatagttgttgctcgtaattccaaacgataattcatgcatgtttcccgaggcacccggcagtggtccagctgaaaaaagtgtttgtgggacgcgggatggtatgtttggcaagtggtttatcagcgccggagtagcagcgcttgtaggaaatatgccggtcgcgatgttccccttaaaacagagtggaatgccgcaaagacctaatccggcgtttccatgacgatcaatttttttggcggttcgccgcaagccatcccagtctgtagccagaagggcatttaatggttttgcagcttggcaacgcgctatcagagtttttactagttctaagcaggagtagtctttccgtctcaggcgttctagggtttgtgctaacgaggtaatgggcaccat

SEQ ID NO: 2 - protein, 466 aa, Agrobacterium tumefaciens
MVPITSLAQTLERLRRKDYSCLELVKTLIARCQAAKPLNALLATDWDGLRRTAKKIDRHGNAGLGLCGIPLCFKGNIATGIFPTSAATPALINHLPNIPSRVPQTLFSAGPLPGASGNMHELSFGITSNNYATGAVRNPWNPSLIPGGSSGGVAAAVASRLMLGGIGTDTGASVRLPAALCGVVGFRPTLARYPRDRIIPVSPTRDTAGIIAQCVADVIILDQVISGRSAKILPMPLKGLRIGLPTTYFYDDLDADVAFAAETTIRLLANRGVTFVEADIPHLEELNSGASLPIALYEFPHALKKYLDDFVGTFLFLTFQRIRSPDVANIVSAQIDGHQISNDEYELARQSFRPRLQATYRNYFRLYQLDAILFPTAPLAAKAIGQESSVIHNGSMMNTFKIYVRNVDPSSNAGLPGLSLPACLTPDRLPVGMEIDGLAGSDHRLLAIGAALEKAINFPSFPDAFN

SEQ ID NO: 3 - DNA, 2268 bp, Agrobacterium tumefaciens
atgtcagcttcacctctccttgataaccagtgcgatcatctcccaaccaaaatggtggatctgacaatggtcgataaggcggatgaattggaccgcagggtttccgatgccttcttagaacgagaagcttctaggggaaggaggattactcaaatctccaccgagtgcagcgctgggttagcttgcaaaaggctggccgatggtcgcttccccgagatctcagctggtggaaaggtagcagttctctccgcttatatctatattggcaaagaaattctggggcggatacttgaatcgaaaccttgggcgcgggcaacagtgagtggtctcgttgccatcgacttggcaccattttgcatggatttctccgaagcacaactaatccaagccctgtttttgctgagcggtaaaagatgtgcaccgattgatcttagtcatttcgtggccatttcaatctctaagactgccggctttcgaaccctgccaatgccgctgtacgagaatggcacgatgaaatgcgttaccgggtttaccataacccttgaaggggccgtgccatttgacatggtagcttatggtcgaaacctgatgctgaagggttcggcaggttcctttccaacaatcgacttgctctacgactacagaccgttttttgaccaatgttccgatagtggacggatcggcttctttccggaggatgttcctaagccgaaagtggcggtcattggcgctggcatttccggactcgtggtggcaaacgaactgcttcatgctggggtagacgatgttacaatatatgaagcaagtgatcgtgttggaggcaagctttggtcacatgctttcagggacgctcctagtgtcgtggccgaaatgggggcgatgcgatttcctcctgctgcattctgcttgtttttcttcctcgagcgttacggcctgtcttcgatgaggccgttcccaaatcccggcacagtcgacacttacttggtctaccaaggcgtccaatacatgtggaaagccgggcagctgccaccgaagctgttccatcgcgtttacaacggttggcgtgcgttcttgaaggacggttttcatgagcgagatattgtgttggcttcgcctgtcgctattactcaggccttgaaatcaggagacattaggtgggctcatgactcctggcaaatttggctgaaccgtttcgggagggagtccttctcttcagggatagagaggatctttctgggcacacatcctcctggtggtgaaacatggagttttcctcatgattgggacctattcaagctaatgggaataggatctggcgggtttggtccagtttttgaaagcgggtttattgagatcctccgcttggtcatcaacggatatgaagaaaatcagcggatgtgccctgaaggaatctcagaacttccacgtcggatcgcatctgaagtggttaacggtgtgtctgtgagccagcgcatatgccatgttcaagtcagggcgattcagaaggaaaagacaaaaataaagataaggcttaagagcgggatatctgaactttatgataaggtggtggtcacatctggactcgcaaatatccaactcaggcattgcctgacatgcgataccaatatttttcaggcaccagtgaaccaagcggttgataacagccatatgacaggatcgtcaaaactcttcctgatgactgaacgaaaattctggttagaccatatcctcccgtcttgtgtcctcatggacgggatcgcaaaagcagtgtattgcctggactatgagtcgcaggatccgaatggtaaaggtctagtgctcatcagttatacatgggaggacgactcccacaagctgttggcggtccccgacaaaaaagagcgattatgtctgctgcgggacgcaatttcgagatctttcccggcgtttgcccagcacctatttcctgcctgcgctgattacgaccaaaatgttattcaacatgattggcttacagacgagaatgccgggggagctttcaaactcaaccggcgtggtgaggatttttattctgaagaacttttctttcaagcactggacacggctaatgataccggagtttacttggcgggttgcagttgttccttcacaggtggatgggtggagggtgctattcagaccgcgtgtaacgccgtctgtgcaattatccacaattgtggaggcattttggcaaagggcaatcctctcgaacactcttggaagagatataactaccgcactagaaattag

SEQ ID NO: 4 - protein, 755 aa, Agrobacterium tumefaciens
MSASALLDNQCDHFSTKMVDLIMVDKADELDRRVSDAFSEREASRGRMITQISGECSAGLACKRLADGRFPEISAGEKVAALSAYIYVGKEILGRILESEPWARARVSGLVAIDLAPFCMDFSEAQLLQTLFLLSGKRCASSDLSHFVAISISKTARSRTLQMPPYEKGTTKRVTGFTLTLEEAVPFDMVAYGRNLMLKASAGSFPTIDLLYDYRSFFDQCSDSGRIGFFPEDVPKPKVAIIGAGISGLVVASELLHAGVDDVTIYEASDRVGGKLWSHAFKDAPSVVAEMGAMRFPPAASCLFFFLERYGLSSMRPFPNPGTVDTNLVYQGLRYMWKAGQQPPKLFHRVYSGWRAFLKDGFHEGDIVLASPVAITQALKSGDIRRAHDSWQTWLNRFGRESFSSAIERIFLGTHPPGGETWSFPHDWDLFKLMGIGSGGFGPVFESGFIEILRLVINGYEENQRMCSEGISELPRRIATQVVNGVSVSQRIRHVQVRAIEKEKTKIKIRLKSGISELYDKVVVTSGLANIQLRHCLTCDTTIFRAPVNQAVDNSHMTGSSKLFLLTERKFWLDHILPSCVLMDGIAKAVYCLDYEPQDPNGKGLVLISYTWEDDSHKLLAVPDKKERFCLLRDAISRSFPAFAQHLVPACADYDQNVVQHDWLTDENAGGAFKLNRRGEDFYSEELFFQALDMTNDTGVYLAGCSCSFTGGWVEGAIQTACNAVCAIIHNCGGILAKDNPLEHSWKRYNYRNRN

SEQ ID NO: 5 - DNA, 663 bp, Agrobacterium fabrum
atgtatcacagtcgaccgatattcaacattatcgacagctcgaatatacaagatcggcgtgaacttaaactcgtcctacgtcacacagagattgcttatcgcagctttgcacaagaggatctcatacctgctcaacgttcctggatgaattccatcattaatactgatgttcccattgatccagctatagatgaagtggtcaagcgcttttgcgaagttgcttgcttgccaggcccagcaggtatacccctgaatattatactcaatgattctctaacgtacgtttattgttcgtttcaagcaatgcgaaagtacgcacacaagcgattttatgacggcgtttccgacgaaggcgtagtcatctccaccgttccaccctatgcggaaggaataacaaaagaaactatgaggtcgtggcacaacaacgtctgtcagaatacaagcaatgaaacacatgatttggatgcttatattgctttccttcctacttcgcttcaaaatccaagtttctcgcatatgaagatcggctgcgatagcttcttagcgccatcccgggttgatcctttctgtgttgaaataatcgcggtcggcaaagccctctttcatgataacgggctgaaaaagaacccaaggtgtggtggtcaatggctcccaccttcttgt

SEQ ID NO: 6 - protein, 221 aa, Agrobacterium fabrum
MYHSRPIFNIIDSSNIQDRRELKLVLRHTEIAYRSFAQEDLIPAQRSWMNSIINTDVPIDPAIDEVVKRFCEVACLPGPAGIPLNIILNDSLTYVYCSFQAMRKYAHKRFYDGVSDEGVVISTVPPYAEGITKETMRSWHNNVCQNTSNETHDLDAYIAFLPTSLQNPSFSHMKIGCDSFLAPSRVDPFCVEIIAVGKALFHDNGLKKNPRCGGQWLPPSC

SEQ ID NO: 7 - protein, 455 aa, Pseudomonas savastanoi
MHEIITLESLCQALADGEIAAAELRERALDTEARLARLNCFIREGDAVSQFGEADHAMKGTPLWGMPVSFKDNICVRGLPLTAGTRGMSGFVSDQDAAIVSQLRALGAVVAGKNNMHELSFGVTSINPHWGTVGNPVAPGYCAGGSSGGSAAAVASGIVPLSVGTDTGGSIRIPAAFCGITGFRPTTGRWSTAGIIPVSHTKDCVGLLTRTAGDAGFLYGLLSGKQQSFPLSRTAPCRIGLPVSMWSDLDGEVERACVNALSLLRKTGFEFIEIDDADIVELNQTLTFTVPLYEFFADLAQSLLSLGWKHGIHHIFAQVDDANVKGIINHHLGEGAIKPAHYLSSLQNGELLKRKMDELFARHNIELLGYPTVPCRVPHLDHADRPEFFSQAIRNTDLASNAMLPSITIPVGPEGRLPVGLSFDALRGRDALLLSRVSAIEQVLGFVRKVLPHTT

SEQ ID NO: 8 - protein, 755 aa, Agrobacterium fabrum
MSASALLDNQCDHFSTKMVDLIMVDKADELDRRVSDAFSEREASRGRRITQISGECSAGLACKRLADGRFPEISAGEKVAALSAYIYVGKEILGRILESEPWARARVSGLVAIDLAPFCMDFSEAQLLQTLFLLSGKRCASSDLSHFVAISISKTARSRTLQMPPYEKGTTKRVTGFTLTLEEAVPFDMVAYGRNLMLKASAGSFPTIDLLYDYRSFFDQCSDSGRIGFFPEDVPKPKVAIIGAGISGLVVASELLHAGVDDVTIYEASDRVGGKLWSHAFKDAPSVVAEMGAMRFPPAASCLFFFLERYGLSSMRPFPNPGTVDTNLVYQGLRYMWKAGQQPPKLFHRVYSGWRAFLKDGFHEGDIVLASPVAITQALKSGDIRRAHDSWQTWLNRFGRESFSSAIERIFLGTHPPGGETWSFPHDWDLFKLMGIGSGGFGPVFESGFIEILRLVINGYEENQRMCSEGISELPRRIATQVVNGVSVSQRIRHVQVRAIEKEKTKIKIRLKSGISELYDKVVVTSGLANIQLRHCLTCDTTIFRAPVNQAVDNSHMTGSSKLFLLTERKFWLDHILPSCVLMDGIAKAVYCLDYEPQDPNGKGLVLISYTWEDDSHKLLAVPDKKERFCLLRDAISRSFPAFAQHLVPACADYDQNVVQHDWLTDENAGGAFKLNRRGEDFYSEELFFQALDMTNDTGVYLAGCSCSFTGGWVEGAIQTACNAVCAIIHNCGGILAKDNPLEHSWKRYNYRNRN

SEQ ID NO: 9 - protein, 467 aa, Agrobacterium tumefaciens
MVAITSLAQSLEHLKRKDYSCLELVETLIARCEAAKSLNALLATDWDGLRRSAKKIDRHGNAGVGLCGIPLCFKANIATGVFPTSAATPALINHLPKIPSRVAERLFSAGALPGASGNMHELSFGITSNNYATGAVRNPWNPDLIPGGSSGGVAAAVASRLMLGGIGTDTGASVRLPAALCGVVGFRPTLGRYPGDRIIPVSPTRDTPGIIAQCVADVVILDRIISGTPERIPPVPLKGLRIGLPTTYFYDDLDADVALAAETTIRLLANKGVTFVEANIPHLDELNKGASFPVALYEFPHALKQYLDDFVKTVSFSDVIKGIRSPDVANIANAQIDGHQISKAEYELARHSFRPRLQATYRNYFKLNRLDAILFPTAPLVARPIGQDSSVIHNGTMLDTFKIYVRNVDPSSNAGLPGLSIPVCLTPDRLPVGMEIDGLADSDQRLLAIGGALEEAIGFRYFAGLPN

SEQ ID NO: 10 - protein, 733 aa, Agrobacterium fabrum
MVDKADELDRRVSDAFSEREASRGRRITQISGECSAGLACKRLADGRFPEISAGEKVAALSAYIYVGKEILGRILESEPWARARVSGLVAIDLAPFCMDFSEAQLLQTLFLLSGKRCASSDLSHFVAISISKTARSRTLQMPPYEKGTTKRVTGFTLTLEEPVPFDMVAYGRNLMLKASAGSFPTIDLLYDYRSFFDQCSDSGRIGFFPEDVPKPKVAIIGAGISGLVVASELLHAAVDDVTIYEASDRVGGKLWSHAFKDAPSVVAEMGAMRFPPAASCLFFFLERYGLSSMRPFPNPGTVDTNLVYQGLRYMWKAGQQPPKLFHRVYSGWRAFLKDGFHEGDIVLASPVAITQALKSGDIRRAHDSWQTWLNRFGRESFSSAIERIFLGTHPPGGETWSFPHDWDLFKLMGIGSGGFGPVFESGFIEILRLVINGYEENQRMCSEGISELPRRIATQVVNGVSVSQRIRHVQVRAIEKEKTKIKIRLKSGISELYDKVVVTSGLANIQLRHCLTCDTTIFRAPVNQAVDNSHMTGSSKLFLLTERKFWLDHILPSCVLMDGIAKAVYCLDYEPQDPNGKGLVLISYTWEDDSHKLLAVPDKKERFCLLRDAISRSFPEFAQHLVPACADYDQNVVQHDWLTDENAGGAFKLNRRGEDFYSEELFFQALDMTNDTGVYLAGCSCSFTGGWVEGAIQTACNAVCAIIHNCGGILAKDNPLEHSWKRYNYRNRN

SEQ ID NO: 11 - protein, 467 aa, Agrobacterium fabrum
MVPITSLAQTLERLRRKDYSCLELVETLIARCQAAKPLNALLATDWDGLRRSAKKIDRHGNAGLGLCGIPLCFKANIATGIFPTSAATPALINHLPKIPSRVAERLFSAGALPGASGNMHELSFGITSNNYATGAVRNPWNPSLIPGGSSGGVAAAVASRLMLGGIGTDTGASVRLPAALCGVVGFRPTLARYPRDRIIPVSPTRDTAGIIAQCVADVIILDQVISGRSAKISPMPLKGLRIGLPTTYFYDDLDADVAFAAETTIRLLANRGVTFVEADIPHLEELNSGASLPIALYEFPHALKKYLDDFVGTVSFSDVIKGIRSPDVANIVSAQIDGHQISNDEYELARQSFRPRLQATYRNYFRLYQLDAILFPTAPLAAKAIGQESSVIHNGSMMNTFKIYVRNVDPSSNAGLPGLSLPACLTPDRLPVGMEIDGLAGSDHRLLAIGAALEKAINFPSFPDAFN

SEQ ID NO: 12 - protein, 749 aa, Agrobacterium rhizogenes
MAGSSFTLPSTGSAPLDMMLIDDSDLLQLGLQQVFSKRYTETPQSRYKLTRRASPDVSSGEGNVHALAFIYVNAETLQMIKNARSLTEANGVKDLVAIDVPPFRNDFSRALLLQVINLLGNNRNADDDLSHFIAVALPNSARSKILTTAPFEGSLSENFRGFPITREGNVACEVLAYGNNLMPKACSDSFPTVDLLYDYGKFFESCAADGRIGYFPEGVTKPKVAIIGAGFSGLVAASELLHAGVDDVTVYEASDRLGGKLWSHGFKSAPNVIAEMGAMRFPRSESCLFFYLKKHGLDSVGLFPNPGSVDTALFYRGRQYIWKAGEEPPELFRRVHHGWRAFLQDGYLHDGVMLASPLAIVDALNLGHLQQAHGFWQSWLTYFERESFSSGIEKMFLGNHPPGGEQWNSLDDLDLFKALGIGSGGFGPVFESGFIEILRLVVNGYEDNVRLSYEGISELPHRIASQVINGRSIRERTIHVQVEQIDREEDKINIKIKGGKVEVYDRVLVTSGFANIEMRHLLTSSNAFFHADVSHAIGNSHMTGASKLFLLTNEKFWLQHHLPSCILTTGVAKAVYCLDYDPRDPSGKGLVLISYTWEDDSHKLLAVPDKRERFASLQRDIGRAFPDFAKHLTPADGNYDDNIVQHDWLTDPHAGGAFKLNRRGNDVYSERLFFQPFDVMHPADDKGLYLAGCSCSFTGGWVHGAIQTACNATCAIIYGSGHLQELIHWRHLKEGNPLAHAWKRYRYQA

SEQ ID NO: 13 - protein, 466 aa, Agrobacterium rhizogenes
MVTLSSITETLKCLRERKYSCFELIETIIARCEAARSLNAFLETDWAHLRWTASKIDQHGGAGVGLAGVPLCFKANIATGRFAATAGTPGLQNHKPKTPAGVARQLLAAGALPGASGNMHELSFGITSNNFATGAVRNPWNPSLIPGGSSGGVAAAVAGRLMLGGVGTDTGASVRLPAALCGVVGFRPTVGRYPTDGIVPVSPTRDTPGVIAQNVPDVILLDGIICGRPPVNQTVRLKGLRIGLPTAYFYNDLEPDVALAAETIIRVLARKDVTFVEADIPDLAHHNEGVSFPTAIYEFPLSLEHYIQNFVEGVSFSEVVRAIRSPDVASILNAQLSDNLISKSEYCLARRFFRPRLQAAYHSYFKAHQLDAILFPTAPLTAKPIGHDLSVIHNGSMTDTFKIFVRNVDPSSNAGLPGLSLPVSLSSNGLPIGMEIDGSASSDERLLAIGLAIEEAIDFRHRPTLS

SEQ ID NO: 14 - protein, 425 aa, Arabidopsis thaliana
MATNNDFGAFIEKVTISPTSTSSSPPSLQGLTFAIKDIFDVEGRVTGFGNPDWLRTHSAATSTAPVVSSLLEAGATALGITIMDEMAYSINGENAHYGTPRNPIAFDRVPGGSSSGSAVAVAARLVDFSIGTDTGGSVRVPASYCGIFGFRPSHGAVSTVGLTPMAQSFDTVGWFARDTATLKRVGCVLLQQHHLNPIEPSQLIIADDCFKLCSVPHDLLVQPLVGSVEKSFGGNTVVKKVNLGEYIGQNVPSLKHFMTSDDVTTQQEFCIPSLMALSSSMRLLQRHEFKINHGAWISSVKPEFGPGISERIEEAIRTSDEKIDHCRSVKSELITALSTLLGEKGVLVIPTVPGPPPHLQANVAALESFRSRAFSLLSIAGVSGFCQVSIPLGLHENLPVSVSLVAKYGSDGFLLSLVDSLAAFI

SEQ ID NO: 15 - protein, 391 aa, Arabidopsis thaliana
MVKLENSRKPEKISNKNIPMSDFVVNLDHGDPTAYEEYWRKMGDRCTVTIRGCDLMSYFSDMTNLCWFLEPELEDAIKDLHGVVGNAATEDRYIVVGTGSTQLCQAAVHALSSLARSQPVSVVAAAPFYSTYVEETTYVRSGMYKWEGDAWGFDKKGPYIELVTSPNNPDGTIRETVVNRPDDDEAKVIHDFAYYWPHYTPITRRQDHDIMLFTFSKITGHAGSRIGWALVKDKEVAKKMVEYIIVNSIGVSKESQVRTAKILNVLKETCKSESESENFFKYGREMMKNRWEKLREVVKESDAFTLPKYPEAFCNYFGKSLESYPAFAWLGTKEETDLVSELRRHKVMSRAGERCGSDKKHVRVSMLSREDVFNVFLERLANMKLIKSIDL

SEQ ID NO: 16 - protein, 388 aa, Arabidopsis thaliana
MMVGCENSKKSDSGSNEDKSLSDDIINLDQGDPTAFQEYWMKKKDRCTVVIPAWDLMSYFSDTKNVCWFLEPELEKAIKALHGAIGNAATEERYIVVGTGSSQLCQAALFALSSLSEVKPVSIVAAVPYYSTYVEEASYLQSTLYKWEGDARTFDKKGPYIELVTSPNNPDGIMREPVVNRREGGKVIHDLAYYWPHYTPITRRQDHDLMLFTFSKITGHAGSRIGWALVKDIEVAKKMVHYLTINSIGVSKESQTRATTILNELTKTCRTQSESFFEYGYEKMKSRWERLREVVESGDAFTLPNYPQDFCNFFGKTLSTSPAFAWLGYKEERDLGSLLKEKKVLTRGGDRCGCNKRYVRVSMLSRDDDFDVSLQRLATIKDLKCVEP

SEQ ID NO: 17 - protein, 1358 aa, Zea mays
MGKEAGAAESSTVVLAVNGKRYEAAGVAPSTSLLEFLRTQTPVRGPKLGCGEGGCGACVVLVSKYDPATDEVTEFSASSCLTLLHSVDRCSVTTSEGIGNTRDGYHPVQQRLSGFHASQCGFCTPGMCMSIFSALVKADNKSDRPDPPAGFSKITTSEAEKAVSGNLCRCTGYRPIVDTCKSFASDVDLEDLGLNCFWKKGEEPAEVSRLPGYNSGAVCTFPEFLKSEIKSTMKQVNDVPIAASGDGWYHPKSIEELHRLFDSSWFDDSSVKIVASNTGSGVYKDQDLYDKYIDIKGIPELSVINKNDKAIELGSVVSISKAIEVLSDGNLVFRKIADHLNKVASPFVRNTATIGGNIMMAQRLPFESDVATVLLAAGSTVTVQVASKRLCFTLEEFLEQPPCDSRTLLLSIFIPEWGSDYVTFETFRAAPRPFGNAVSYVNSAFLARTSGSLLIEDICLAFGAYGVDHAIRAKKVEDFLKGKSLSSFVILEAIKLLKDTVSPSEGTTHHEYRVSLAVSFLFSFLSSLANSSSAPSNIDTPNGSYTHETGSNVDSPERHIKVDSNDLPIRSRQEMVFSDEYKPVGKPIKKVGAEIQASGEAVYVDDIPAPKDCLYGAFIYSTHPHAHVRSINFKSSLASQKVITVITAKDIPSGGENIGSSFLMQGEALFADPIAEFAGQNIGVVIAETQRYANMAAKQAVVEYSTENLQPPILTIEDAIQRNSYIQIPPFLAPKPVGDYNKGMAEADHKILSAEVKLESQYYFYMETQAALAIPDEDNCITIYSSTQMPELTQNLIARCLGIPFHNVRVISRRVGGGFGGKAMKATHTACACALAAFKLRRPVRMYLDRKTDMIMAGGRHPMKAKYSVGFKSDGKITALHLDLGINAGISPDVSPLMPRAIIGALKKYNWGTLEFDTKVCKTNVSSKSAMRAPGDVQGSFIAEAIIEHVASALALDTNTVRRKNLHDFESLEVFYGESAGEASTYSLVSMFDKLALSPEYQHRAAMIEQFNSSNKWKKRGISCVPATYEVNLRPTPGKVSIMNDGSIAVEVGGIEIGQGLWTKVKQMTAFGLGQLCPDGGECLLDKVRVIQADTLSLIQGGMTAGSTTSETSCETVRQSCVALVEKLNPIKESLEAKSNTVEWSALIAQASMASVNLSAQPYWTPDPSFKSYLNYGAGTSEVEVDILTGATTILRSDLVYDCGQSLNPAVDLGQIEGCFVQGIGFFTNEDYKTNSDGLVIHDGTWTYKIPTVDNIPKEFNVEMFNSAPDKKRVLSSKASGEPPLVLATSVHCAMREAIRAARKEFSVSTSPAKSAVTFQMDVPATMPVVKELCGLDVVERYLENVSAASAGPNTAKA

SEQ ID NO: 18 - protein, 1368 aa, Arabidopsis thaliana
MGEKAIDEDKVEAMKSSKTSLVFAINGQRFELELSSIDPSTTLVDFLRNKTPFKSVKLGCGEGGCGACVVLLSKYDPLLEKVDEFTISSCLTLLCSIDGCSITTSDGLGNSRVGFHAVHERIAGFHATQCGFCTPGMSVSMFSALLNADKSHPPPRSGFSNLTAVEAEKAVSGNLCRCTGYRPLVDACKSFAADVDIEDLGFNAFCKKGENRDEVLRRLPCYDHTSSHVCTFPEFLKKEIKNDMSLHSRKYRWSSPVSVSELQGLLEVENGLSVKLVAGNTSTGYYKEEKERKYERFIDIRKIPEFTMVRSDEKGVELGACVTISKAIEVLREEKNVSVLAKIATHMEKIANRFVRNTGTIGGNIMMAQRKQFPSDLATILVAAQATVKIMTSSSSQEQFTLEEFLQQPPLDAKSLLLSLEIPSWHSAKKNGSSEDSILLFETYRAAPRPLGNALAFLNAAFSAEVTEALDGIVVNDCQLVFGAYGTKHAHRAKKVEEFLTGKVISDEVLMEAISLLKDEIVPDKGTSNPGYRSSLAVTFLFEFFGSLTKKNAKTTNGWLNGGCKEIGFDQNVESLKPEAMLSSAQQIVENQEHSPVGKGITKAGACLQASGEAVYVDDIPAPENCLYGAFIYSTMPLARIKGIRFKQNRVPEGVLGIITYKDIPKGGQNIGTNGFFTSDLLFAEEVTHCAGQIIAFLVADSQKHADIAANLVVIDYDTKDLKPPILSLEEAVENFSLFEVPPPLRGYPVGDITKGMDEAEHKILGSKISFGSQYFFYMETQTALAVPDEDNCMVVYSSTQTPEFVHQTIAGCLGVPENNVRVITRRVGGGFGGKAVKSMPVAAACALAASKMQRPVRTYVNRKTDMITTGGRHPMKVTYSVGFKSNGKITALDVEVLLDAGLTEDISPLMPKGIQGALMKYDWGALSFNVKVCKTNTVSRTALRAPGDVQGSYIGEAIIEKVASYLSVDVDEIRKVNLHTYESLRLFHSAKAGEFSEYTLPLLWDRIDEFSGFNKRRKVVEEFNASNKWRKRGISRVPAVYAVNMRSTPGRVSVLGDGSIVVEVQGIEIGQGLWTKVKQMAAYSLGLIQCGTTSDELLKKIRVIQSDTLSMVQGSMTAGSTTSEASSEAVRICCDGLVERLLPVKTALVEQTGGPVTWDSLISQAYQQSINMSVSSKYMPDSTGEYLNYGIAASEVEVNVLTGETTILRTDIIYDCGKSLNPAVDLGQIEGAFVQGLGFFMLEEFLMNSDGLVVTDSTWTYKIPTVDTIPRQFNVEILNSGQHKNRVLSSKASGEPPLLLAASVHCAVRAAVKEARKQILSWNSNKQGTDMYFELPVPATMPIVKEFCGLDVVEKYLEWKIQQRKNV

SEQ ID NO: 19 - protein, 514 aa, Oryza sativa
MGSLDTNPTAFSAFPAGEGETFQPLNADDVRSYLHKAVDFISDYYKSVESMPVLPNVKPGYLQDELRASPPTYSAPFDVTMKELRSSVVPGMTHWASPNFFAFFPSTNSAAAIAGDLIASAMNTVGFTWQASPAATEMEVLALDWLAQMLNLPTSFMNRTGEGRGTGGGVILGTTSEAMLVTLVAARDAALRRSGSDGVAGLHRLAVYAADQTHSTFFKACRLAGFDPANIRSIPTGAETDYGLDPARLLEAMQADADAGLVPTYVCATVGTTSSNAVDPVGAVADVAARFAAWVHVDAAYAGSACICPEFRHHLDGVERVDSISMSPHKWLMTCLDCTCLYVRDTHRLTGSLETNPEYLKNHASDSGEVTDLKDMQVGVGRRFRGLKLWMVMRTYGVAKLQEHIRSDVAMAKVFEDLVRGDDRFEVVVPRNFALVCFRIRAGAGAAAATEEDADEANRELMERLNKTGKAYVAHTVVGGRFVLRFAVGSSLQEEHHVRSAWELIKKTTTEMMN

SEQ ID NO: 20 - protein, 497 aa, Oryza sativa
MEGVGGGGGGEEWLRPMDAEQLRECGHRMVDFVADYYKSIEAFPVLSQVQPGYLKEVLPDSAPRQPDTLDSLFDDIQQKIIPGVTHWQSPNYFAYYPSNSSTAGFLGEMLSAAFNIVGFSWITSPAATELEVIVLDWFAKMLQLPSQFLSTALGGGVIQGTASEAVLVALLAARDRALKKHGKHSLEKLVVYASDQTHSALQKACQIAGIFSENVRVVIADCNKNYAVAPEAVSEALSIDLSSGLIPFFICATVGTTSSSAVDPLPELGQIAKSNDMWFHIDAAYAGSACICPEYRHHLNGVEEADSFNMNAHKWFLTNFDCSLLWVKDRSFLIQSLSTNPEFLKNKASQANSVVDFKDWQIPLGRRFRSLKLWMVLRLYGVDNLQSYIRKHIHLAEHFEQLLLSDSRFEVVTPRTFSLVCFRLVPPTSDHENGRKLNYDMMDGVNSSGKIFLSHTVLSGKFVLRFAVGAPLTEERHVDAAWKLLRDEATKVLGKMV

SEQ ID NO: 21 - DNA, 720 bp, Agrobacterium fabrum
atggatctgcgtctaattttcggtccaacttgcacaggaaagacgtcgaccgcggtagctcttgcccagcagactgggcttccagtcctttcgctcgatcgggtccaatgttgtcctcagctgtcaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattcttgagggaggatctatctcgttgctcaagtgcatggcgcaaagcagttattggagtgcggattttcgttggcatattattcgccacgagttagcagacgaggagaccttcatgaacgtggccaaggccagagttaagcagatgttacgccctgctgcaggcctttctattatccaagagttggttgatctttggaaagagcctcggctgaggcccatactgaaagagatcgatggatatcgatatgccatgttgtttgctagccagaaccagatcacatccgatatgctattgcagcttgacgcagatatggaggataagttgattcatgggatcgctcaggagtatctcatccatgcacgccgacaagaacagaaattccctcgagttaacgcagccgcttacgacggattcgaaggtcatccattcggaatgtat

SEQ ID NO: 22 - protein, 240 aa, Agrobacterium fabrum
MDLRLIFGPTCTGKTSTAVALAQQTGLPVLSLDRVQCCPQLSTGSGRPTVEELKGTSRLYLDDRPLVKGIIAAKQAHERLMGEVYNYEAHGGLILEGGSISLLKCMAQSSYWSADFRWHIIRHELADEETFMNVAKARVKQMLRPAAGLSIIQELVDLWKEPRLRPILKEIDGYRYAMLFASQNQITSDMLLQLDADMEDKLIHGIAQEYLIHARRQEQKFPRVNAAAYDGFEGHPFGMY

SEQ ID NO: 23 - protein, 243 aa, Agrobacterium rhizogenes
MLLYLIYGPTCSGKTDIAIQIAQKTGWPVVALDRVQCCPQIATGSGRPLPSELQSTRRIYLDSRRLTKGIIDAEGAHRRLILEVDWQESEEGLILEGGSVSLLNCMAKSPYWKSGFQWHVKRLRLGDSDAFLARAKQRVTEMFAIREDRPSLLEELAELWNYPATRPILEDIDGYRCAIRFARKHDLAINQLPDIDAERQQDLIEAIAKEYLEHAIMQERDFPQWPEDGARQPVGPATLMRIQ

SEQ ID NO: 24 - DNA, 282 bp, Artificial
  polynucleotide Jaburetox
atgggtccagttaatgaagccaattgtaaagcagctatggagattgtgtgcagaagggaatttggacataaggaagaagaagatgcaagtgagggtgttaccacaggagaccctgattgtcctttcaccaaagccattcctcgtgaagaatatgctaacaagtatggtccgactattggtgacaaaatccgtcttggtgacactgatttgattgctgaaattgaaaaggattttgccctttatggtgatgaaagtgtttttggaggtggaaaagttatatag

SEQ ID NO: 25 - protein, 93 aa, Artificial
  polypeptide Jaburetox
MGPVNEANCKAAMEIVCRREFGHKEEEDASEGVTTGDPDCPFTKAIPREEYANKYGPTIGDKIRLGDTDLIAEIEKDFALYGDESVFGGGKVI

SEQ ID NO: 26 - DNA, 36 bp, Aedes aegypti
atgtacgatcctgctccacccccaccgccaccttaa

SEQ ID NO: 27 - protein, 10 aa, Aedes aegypti
YDPAPPPPPP

SEQ ID NO: 28 - protein, 6 aa, Neobellieria bullata
NPTNLH

SEQ ID NO: 29 - DNA, 45 bp, Oncopeltus fasciatus
atggtcgataagccaccgtatttaccgaggccgagaccgccgtaa

SEQ ID NO: 30 - protein, 13 aa, Oncopeltus fasciatus
VDKPPYLPRPRPP

SEQ ID NO: 31 - protein, 19 aa, Artificial
  Oncocin analogue
  MISC_FEATURE (15)..(15): wherein Xaa is ornithine
  MISC_FEATURE (19)..(19): wherein Xaa is ornithine
VDKPPYLPRPRPPRXIYNX

SEQ ID NO: 32 - protein, 19 aa, Artificial
  Oncocin analogue
  MISC_FEATURE (15)..(15): wherein r is D-arginine
  MISC_FEATURE (19)..(19): wherein r is D-arginine
VDKPPYLPRPRPPRRIYNR

SEQ ID NO: 33 - DNA, 1863 bp, Bacillus thuringiensis
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagacgcgtattcaattcaatgacatgaacagtgcccttacaaccgctattcctcttttggcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtacgctggtacaatacgggattagaacgtgtatggggaccggattctagagattgggtaaggtacaatcaatttaggagagaattaacgctaactgtattagatatcgttgctctgttcccgaattatgatagtagaagatatccaattcgaacagtttcccaattaacaagagaaatttatacaaacccagtattagaaaattttgatggtagttttcgaggctcggctcagggcatagaaagaagtattaggagtccacatttgatggatatacttaacagtataaccatctatacggatgctcataggggttattattattggtcagggcatcaaataatggcttctcctgtcggtttttcggggccagaattcacgtttccgctatatggaaccatgggaaatgcagctccacaacaacgtattgttgctcaactaggtcagggcgtgtatagaacattatcctctactttttatagaagaccttttaatatagggataaataatcaacaactatctgttcttgacgggacagaatttgcttatggaacctcctcaaatttgccatccgctgtatacagaaaaagcggaacggtagattcgctggatgaaataccaccacagaataacaacgtgccacctaggcaaggatttagtcatcgattaagccatgtttcaatgtttcgttcaggctctagtaatagtagtgtaagtataataagagctcctatgttctcttggatacatcgtagtgctgaatttaataatataattgcatcggatagtattactcaaatccctgcagtgaagggaaactttctttttaatggttctgtaatttcaggaccaggatttactggtggggacttagttagattaaatagtagtggaaataacattcagaatagagggtatattgaagttccaattcacttcccatcgacatctaccagatatcgagttcgtgtacggtatgcttctgtaaccccgattcacctcaacgttaattggggtaattcatccattttttccaatacagtaccagctacagctacgtcattagataatctacaatcaagtgattttggttattttgaaagtgccaatgcttttacatcttcattaggtaatatagtaggtgttagaaattttagtgggactgcaggagtgataatagacagatttgaatttattccagttactgcaacactcgaggctgaatataatctggaaagatag

SEQ ID NO: 34 - protein, 620 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREETRIQFNDMNSALTTAIPLLAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTFYRRPFNIGINNQQLSVLDGTEFAYGTSSNLPSAVYRKSGTVDSLDEIPPQNNNVPPRQGFSHRLSHVSMFRSGSSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAEYNLER

SEQ ID NO: 35 - protein, 16 aa, Bacillus brevis
AVGALAVVVWLWLWLW

SEQ ID NO: 36 - protein, 23 aa, Artificial
  Magainin 2
GIGKFLHSAKKFGKAFVGEIMNS

SEQ ID NO: 37 - protein, 35 aa, Artificial
  cathelicidin
LGDFFRKSKEKIGKEFKRIVQRIKFLRNLVPRTES

SEQ ID NO: 38 - protein, 20 aa, Pyrrhocoris apterus
VDKGSYLPRPTPPRPIYNRN

SEQ ID NO: 39 - protein, 34 aa, Lactococcus lactis
ITSISLCTPGCKTGALMGCNMKTATCHCSIHVSK

SEQ ID NO: 40 - protein, 30 aa, Homo sapiens
ACYCRIPACIAGERRYGTCIYQGRLWAFCC

SEQ ID NO: 41 - protein, 38 aa, Bos taurus
NPVSCVRNKGICVPIRCPGSMKQIGTCVGRAVKCCRKK

SEQ ID NO: 42 - protein, 40 aa, Pseudoplectania nigrella
GFGCNGPWDEDDMQCHNHCKSIKGYKGGYCAKGGFVCKCY

SEQ ID NO: 43 - protein, 10 aa, Paenibacillus polymyxa
  MISC_FEATURE (1)..(1): wherein Xaa is 2,4-diaminobutanoic acid
  MISC_FEATURE (3)..(4): wherein Xaa is 2,4-diaminobutanoic acid
  MISC_FEATURE (8)..(9): wherein Xaa is 2,4-diaminobutanoic acid
XTXXKLLXXT

SEQ ID NO: 44 - protein, 13 aa, Streptomyces roseosporus
WNDTGKDADGSEY

SEQ ID NO: 45 - protein, 21 aa, Escherichia coli
VGIGTPIFSYGGGAGHVPEYF

SEQ ID NO: 46 - protein, 18 aa, Trichoderma permeabilises
  MISC_FEATURE (2)..(2): wherein B is alpha-aminoisobutyric acid
  MISC_FEATURE (4)..(4): wherein B is alpha-aminoisobutyric acid
  MISC_FEATURE (7)..(7): wherein B is alpha-aminoisobutyric acid
  MISC_FEATURE (9)..(9): wherein B is alpha-aminoisobutyric acid
  MISC_FEATURE (12)..(12): wherein B is alpha-aminoisobutyric acid
  MISC_FEATURE (15)..(16): wherein B is alpha-aminoisobutyric acid
PBABAQBVBGLBPVBBEQ

SEQ ID NO: 47 - protein, 11 aa, Bacillus brevis
SVKLFPVKLFP

SEQ ID NO: 48 - protein, 35 aa, Bacillus subtilis
NKGCATCSIGAACLVDGPIPDFEIAGATGLFGLWG

SEQ ID NO: 49 - protein, 29 aa, Oldenlandia affinis
GLPVCGETCVGGTCNTPGCTCSWPVCTRN

SEQ ID NO: 50 - protein, 18 aa, Rhesus macaques
GFCRCLCRRGVCRCICTR

SEQ ID NO: 51 - protein, 221 aa, Ficus carica
LPETVDWRIQGAVNPIRNQGRCGSCWAFSVVVVVEGITKIVTDELPSLSEQQLVDCATSYKNLGCSGGWMKAYDYIIKNGGITSQSNYPYTAKKGECNKDLASQIVATIDSYEHVPRNNENALKNAVANQPVSVTIEAGGRAFELYKSGVFVGSCGTKLDHAVVAIGYGSENDVDYWLVRNSWGTNWGERGYIKLQRNVAEPTGKCGIAMQSTYPVKKTSA

SEQ ID NO: 52 - protein, 57 aa, Nicotiana tabacum
MASSVLSSAAVATRSNVAQANMVAPFTGLKSAASFPVSRKQNLDITSIASNGGRVQC

SEQ ID NO: 53 - protein, 26 aa, Arabidopsis thaliana
MRILPKSGGGALCLLFVFALCSVAHS

SEQ ID NO: 54 - protein, 9 aa, Unknown
  PTS-2
  MISC_FEATURE (3)..(7): wherein Xaa can be any naturally occurring amino acid
RLXXXXXHL

SEQ ID NO: 55 - protein, 13 aa, Unknown
  PTS-2
MRLSIHAEHLSKL

SEQ ID NO: 56 - protein, 85 aa, Arabidopsis thaliana
MLRTVSCLASRSSSSLFFRFFRQFPRSYMSLTSSTAALRVPSRNLRRISSPSVAGRRLLLRRGLRIPSAAVRSVNGQFSRLSVRA

SEQ ID NO: 57 - protein, 35 aa, Chlamydomonas reinhardtii
MALVARPVLSARVAASRPRVAARKAVRVSAKYGEN

SEQ ID NO: 58 - protein, 29 aa, Chlamydomonas reinhardtii
MQALSSRVNIAAKPQRAQRLVVRAEEVKA

SEQ ID NO: 59 - protein, 35 aa, Chlamydomonas reinhardtii
MQTLASRPSLRASARVAPRRAPRVAVVTKAALDPQ

SEQ ID NO: 60 - protein, 31 aa, Chlamydomonas reinhardtii
MQALATRPSAIRPTKAARRSSVVVRADGFIG

SEQ ID NO: 61 - protein, 51 aa, Chlamydomonas reinhardtii
MAFALASRKALQVTCKATGKKTAAKAAAPKSSGVEFYGPNRAKWLGPYSEN

SEQ ID NO: 62 - protein, 50 aa, Chlamydomonas reinhardtii
MAAVIAKSSVSAAVARPARSSVRPMAALKPAVKAAPVAAPAQANQMMVWT

SEQ ID NO: 63 - protein, 40 aa, Chlamydomonas reinhardtii
MAAMLASKQGAFMGRSSFAPAPKGVASRGSLQVVAGLKEV

SEQ ID NO: 64 - protein, 4 aa, Arabidopsis thaliana
CVVQ

SEQ ID NO: 65 - protein, 9 aa, Unknown
  PTS-2
RLAVAVAHL

(19) United States
(12) Patent Application Publication
(10) Pub. No.: US 2022/0304273 A1
(43) Pub. Date: Sep. 29, 2022

(54) COMPOSITIONS AND METHODS FOR MODIFYING A PLANT CHARACTERISTIC WITHOUT MODIFYING THE PLANT GENOME

(71) Applicants: THE UNITED STATES OF AMERICA, AS REPRESENTED BY THE SECRETARY OF AGRICULTURE, Washington, DC (US); AGROSOURCE, INC., Jupiter, FL (US)

(72) Inventors: Robert G. Shatters, Fort Pierce, FL (US); Eddie W. Stover, Fort Pierce, FL (US); Randall P. Niedz, Fort Pierce, FL (US); Michelle L. Heck, lthaca, NY (US); Marco Pitino, Port St. Lucie, FL (US); Magali Ferrari Grando, Fort Pierce, FL (US); Joseph Krystel, Fort Pierce, FL (US)

(21) Appl. No.: 17/635,494

(22) PCT Filed: Sep. 18, 2020
(86) PCT No.: PCT/US2020/051356

§ 371 Date: Feb. 15, 2022

Related U.S. Application Data
(60) Provisional application No. 62/903,183, filed on Sep. 20, 2019.

(51) Int. Cl. A01H 17/00; C12N 1/20; C12N 9/02

(52) U.S. Cl. CPC A01H 17/00; C12N 1/20; C12Y 102/03007; C12N 9/0008

(57) ABSTRACT

The invention relates to methods and compositions for modifying a characteristic of a plant without modifying the plant's genome using one or more cells comprising one or more phytohormone genes and at least one polynucleotide of interest, which one or more phytohormone genes and the at least one polynucleotide of interest are expressed in the one or more cells.

### STATEMENT REGARDING ELECTRONIC FILING OF A SEQUENCE LISTING

A Sequence Listing in ASCII text format, submitted under 37 C.F.R. § 1.821, entitled 1554-3WO_ST25.txt, 126,446 bytes in size, generated on Sep. 17, 2020 and filed via EFS-Web, is provided in lieu of a paper copy. This Sequence Listing is hereby incorporated herein by reference into the specification for its disclosures.

### STATEMENT OF PRIORITY

This application claims the benefit, under 35 U.S.C. § 119 (e), of U.S. Provisional Application No. 62/903,183 filed on Sep. 20, 2019, the entire contents of which is incorporated by reference herein.

### FIELD OF THE INVENTION

The invention relates to symbiont forming inoculum and symbionts that comprise polynucleotides encoding one or more phytohormone genes and at least one polynucleotide of interest, which can be used to modify a characteristic of a host plant without modifying the host plant's genome.

### BACKGROUND OF THE INVENTION

Bacteria in the genus Agrobacterium have been studied for decades as a plant pathogen causing crown gall disease. The disease results in the formation of a plant mass (or gall) growing on the plant at the site of infection by Agrobacterium spp. Galls that occur on mature plants can result in few or no phenotypic responses or effects on plant growth depending on the pathogen and host genotype and age of the host on infection. However, galls on younger plants can severely adversely affect growth and other characteristics of the plant. Gall formation is induced as a result of the bacterium's ability to enter wound sites in plants and transfer a portion of DNA (called T-DNA, or transfer DNA, that is located on an Agrobacterium spp. plasmid called the Ti-plasmid) to the neighboring plant cells. Once inside the plant cell, the T-DNA is directed toward the nucleus where it is inserted in the genome of the plant.

In the 1940's, before Agrobacterium spp. was known to transfer DNA into the plant genome, researchers discovered that the bacterium could stably alter plant cells to become “immortal” and grow in in vitro culture independent of the need for plant hormones. It is now known that tumor formation is the result of the T-DNA containing genes for phytohormone synthesis that are expressed when inserted into the plant cells genome. The subsequently produced phytohormones cause the plant cell to initiate cell division no longer under the control of the plant-produced cell division signals.

Since the 1980's, Agrobacterium spp. has been used in research and applications to transform entire plants due to its ability to insert T-DNA into the targeted plant's genome. Such T-DNA can be engineered to deliver genes that impart a desired trait into the target plant. To achieve the transformation, “disarmed” strains of Agrobacterium spp. were developed which do not form galls and thus the resulting plant only realizes the direct effect of the genes of interest delivered to produce a desired phenotype.

However, transgenesis and transformed plants are not always desirable for several reasons. First, plant transformation is a laborious process with a low frequency of successful transformation of plant germline tissue. Successful transgenic gene expression in plants may be influenced by the expression of neighboring genes and the copy number of transgene insertion into the plant genome. Second, the traditional process of transgenesis does not facilitate a real-time response to an environmental stress, pest, or pathogen. Instead, the process is done in a lab setting, and thus, cannot be used as a dynamic response to temporal stimuli. Third, the transformed genome with heterogeneous DNA may be present in the harvested material from the plant (for example, in the harvested fruit or vegetable) and there is a desire in most markets to not have such transformed DNA present in the resulting edible foods. In addition, there is a desire not to have pollen from transgenic plants in the environment.

Thus, there is a need for a method which is capable of imparting one or more desired traits into a target plant without introducing heterogeneous DNA into the entire plant.

### SUMMARY OF THE INVENTION

One aspect of the invention provides a symbiont forming inoculum comprising a polynucleotide encoding a phytohormone biosynthetic enzyme and a polynucleotide of interest, wherein the phytohormone biosynthetic enzyme is at least one cytokinin biosynthetic enzyme and/or an auxin biosynthetic enzyme.

A second aspect provides a symbiont comprising a plant cell comprising and expressing a polynucleotide encoding a phytohormone biosynthetic enzyme and a polynucleotide of interest, wherein the phytohormone biosynthetic enzyme is at least one cytokinin biosynthetic enzyme and/or an auxin biosynthetic enzyme and the plant cell of the symbiont autonomously divides. In some aspects, the plant cell comprises at least two cells.

A third aspect of the invention provides a method of producing a symbiont forming inoculum, the method comprising: introducing into a cell a polynucleotide encoding a phytohormone biosynthetic enzyme and a polynucleotide of interest or introducing a polynucleotide encoding a phytohormone biosynthetic enzyme into a transgenic cell that comprises a polynucleotide of interest, wherein the phytohormone biosynthetic enzyme is at least one cytokinin biosynthetic enzyme and/or an auxin biosynthetic enzyme, thereby producing the symbiont forming inoculum.

A fourth aspect of the invention provides a method of producing a symbiont forming inoculum, the method comprising (a) (i) introducing into/onto at least one site (e.g., 1, 2, 3, 4, 5, 6, 7, 8, 9, 10, or more) on a plant (or a part thereof (e.g., explant)) a polynucleotide encoding a phytohormone biosynthetic enzyme and a polynucleotide sequence of interest or transplanting a plant cell or inoculating bacterial cell comprising the same (e.g., a polynucleotide encoding a phytohormone biosynthetic enzyme and a polynucleotide sequence of interest) onto at least one site on the plant (or a part thereof), or (ii) introducing a polynucleotide encoding a phytohormone biosynthetic enzyme into/onto at least one site on a plant (or a part thereof) or transplanting a plant cell or inoculating bacterial cell comprising the same onto at least one site on the plant (or a part thereof), wherein the plant (or a part thereof) of (ii) comprises a polynucleotide sequence of interest, wherein the phytohormone biosynthetic enzyme of (a) is at least one cytokinin biosynthetic enzyme and/or an auxin biosynthetic enzyme, thereby producing a symbiont on the plant (or part thereof) that comprises the polynucleotide encoding a phytohormone biosynthetic enzyme and the polynucleotide sequence of interest; and (b) selecting one or more cells (e.g., 1, 2, 3, 4, 5, 6, 7, 8, 9, 10, 20, 30, 40, 50, 60, 70, 80, 90, 100 or more cells) from the symbiont on the plant, to provide one or more cells comprising the polynucleotide encoding a phytohormone biosynthetic enzyme and the polynucleotide sequence of interest, thereby producing the symbiont forming inoculum.

A fifth aspect of the invention provides a method of modifying a host plant characteristic without modifying the host plant genome, the method comprising transplanting the symbiont forming inoculum of the invention or the symbiont of the invention onto at least one site (e.g., 1, 2, 3, 4, 5, 6, 7, 8, 9, 10 or more sites) on a host plant; and culturing the symbiont forming inoculum or symbiont at the at least one site on the host plant to form a symbiont on the host plant at the at least one site, wherein the polynucleotide of interest is expressed in the symbiont on the host plant and an expression product of the polynucleotide of interest and/or a product made using the expression product of the polynucleotide of interest is transported into the host plant, thereby modifying the host plant characteristic.

A sixth aspect of the invention provides a method of producing a biomolecule or a bioactive molecule, comprising providing a symbiont of the invention, wherein the polynucleotide of interest encodes a bioactive molecule and collecting the bioactive molecule produced by the symbiont; and/or providing a host plant of the invention, wherein the polynucleotide of interest encodes a bioactive molecule and collecting the bioactive molecule produced in the symbiont and host plant.

A seventh aspect of the invention provides a method of delivering a compound of interest to a host plant, comprising transplanting onto at least one site (e.g., 1, 2, 3, 4, 5, 6, 7, 8, 9, 10 or more sites) on a host plant a symbiont forming inoculum of the invention or a symbiont of the invention and culturing the symbiont forming inoculum or symbiont at the at least one site on the host plant to form a symbiont on the host plant at the at least one site, wherein the polynucleotide of interest is expressed in the symbiont and an expression product of the polynucleotide of interest and/or a product made using the expression product of the polynucleotide of interest is transported into the host plant, thereby delivering the compound of interest to a plant.

An eighth aspect of the invention provides a method of producing a host plant comprising a modified characteristic(s) without modifying the host plant's genotype, comprising: transplanting onto at least one site (e.g., 1, 2, 3, 4, 5, 6, 7, 8, 9, 10 or more sites) on a host plant a symbiont forming inoculum of the invention or a symbiont of the invention; and culturing the symbiont forming inoculum or symbiont at the at least one site on the host plant to form a symbiont on the host plant at the at least one site, wherein the polynucleotide of interest is expressed in the symbiont and an expression product of the polynucleotide of interest and/or a product made using the expression product of the polynucleotide of interest is transported into the host plant, thereby producing the plant comprising a modified phenotype without a modified genotype.

Further provided are symbiont forming inoculum, symbionts, host plants, plants and cells and/or protoplasts produced by the methods of the invention as well as the nucleic acids, expression cassettes and vectors comprising the same for carrying out the methods.

These and other aspects of the invention are set forth in more detail in the description of the invention below.

### BRIEF DESCRIPTION OF THE DRAWINGS

FIG. 1. Demonstration of symbiont formation using co-inoculation and single-strain inoculation (Agrobacterium), and gene gun methods of deliver genes encoding phytohormone production (PH) and polynucleotides of interest (POI) into plant cells.

FIG. 2. Example of a plasmid map (“pSYM”) encoding at least one phytohormone polypeptide (plant growth regulator (PGR) expression cassette) and a polynucleotide of interest (POI). AscI, XmaI and SpeI are restriction sites, NosT is a nopaline synthase terminator, and Kan represents kanamycin selection markers.

FIG. 3. Illustration of different example pathways for generating a symbiont. DNA delivery can be done using any method, for example, bacteria, bombardment, electroporation, whiskers, protoplast fusion, and the like. “Activated Tissue” is tissue that has been immortalized with phytohormone (PH) genes; “Mixed Culture” is a collection of cells with a variety of different gene insertions and expression; “Symbiont Forming Inoculum” is an inoculum that may be used to form a symbiont on a plant (e.g., DNA, bacterial cells, plant cells, and the like); Symbiont is plant tissue (e.g., one or more plant cells) having both PH gene(s) and polynucleotide(s) of interest (POI)), optionally located on a plant.

FIG. 4. Symbiont formation on citrus after 60 days post-inoculation. Panels A and B show symbionts formed using co-inoculation (e.g., more than one Agrobacterium strain). Panels C and D show symbionts formed using single-strain inoculation.

FIG. 5. Examples of inoculation techniques with Agrobacterium spp. Panel A shows the use of tweezers on Citrus. Panels B and C show the use of two example needle types on tomatoes plants, a tattoo needle (Panel B) and a hypodermic needle (Panel C).

FIG. 6. Examples of symbionts on different crop types. Panel A. Pecan; Panel B. Tomato; Panel C. Citrus; Panel D. Tobacco (Nicotiana benthamiana).

FIG. 7. Symbiont forming inoculum (in the form of plant callus tissue) growing on a solid media exhibiting a high level of mCherry production.

FIG. 8. Examples of different types of symbiont forming inoculum grown on solid media (Panels A and B) and liquid media (Panels C and D). Tomato (Panels A and C) and Citrus (Panels B and D).

FIG. 9. Examples of symbiont transplantation on Citrus at 1 and 6 weeks (Panels A and B); and tomato at 2 and 6 weeks (Panels D and E). Panels C and F illustrate the vascularization (C) and Green Fluorescent Protein (GFP) production (Panel F) of a transplanted citrus symbiont. Silicon tape (Panel A) or parafilm (Panel D) is used initially to control humidity at the transplantation site.

FIG. 10. Plasmid map of an example pSYM plasmid having multiple (e.g., “stacked”) polynucleotides of interest (POIs) encoding product(s) of interest.

FIG. 11. Examples of symbiont stacking and POI stacking. Autofluorescence (Panel A) and GFP fluorescence (Panel B) of multiple individual small GFP pSYM on tomato. Panels C-E show stacking of two pSYM plasmids with different polynucleotides of interest (POI) on a single plant: autofluorescence (Panel C), mCherry (Panel D) and GFP (Panel E). Panels F-H show stacking of multiple polynucleotides of interest (POI) in a single pSYM Autofluorescence (Panel F), mCherry (Panel G), GFP (Panel H).

FIG. 12. Tomato and citrus symbionts expressing high levels of green fluorescent protein (GFP). Panel A. GFP protein accumulation in tomato symbiont at such levels it can be viewed with the naked eye. Panels B and C show a cross-section of a citrus symbiont established with single-strain inoculation (Agrobacterium spp.). Arrows indicate areas with high accumulation of GFP inside the symbiont

FIG. 13. Immunodetection of mCherry produced in a symbiont formed using single-strain inoculation (Agrobacterium spp.) on tomato using western blot detection method. mCherry was detectable out to 10⁷ dilution of the original protein extract

FIG. 14. Microscopic view of a symbiont containing a polynucleotide of interest encoding mCherry florescent protein on a tomato plant. Panel A shows UV autofluorescence of growing plant vascular tissue beginning to extend into the symbiont tissue. Panel B shows Red mCherry production and accumulation inside the symbiont as well as accumulation in the vascular tissue that has grown into the symbiont tissue. Panel C shows vascular tissue developing in the symbiont. Panel D shows mCherry fluorescence detection in the stem vascular tissue demonstrating the export of mCherry protein outside of the symbiont.

FIG. 15. Cross-section of a tomato stem 1-2 cm above a symbiont expressing GFP illustrating export of POI products. Arrows indicate GFP accumulation.

FIG. 16. Detection of mCherry in different parts of tomato host plant with two attached symbionts, both containing polynucleotides coding for mCherry protein production. Panel A: Symbiont 1; Panel B: Stem above Symbiont 1; Panel C: Symbiont 2; Panel D: Stem above Symbiont 2; Panel E: Below Symbiont 1; Panel F: Below Symbiont 2; Panel G: Control.

FIG. 17. PCR detection of the polynucleotide of interest (GFP, GFP+) in a symbiont (“Sym”) versus the tomato host plant stem illustrating that only the symbiont is genetically transformed “GFP+” is GFP linked to a secretory pathway targeting sequence (endoplasmic reticulum (ER) targeting sequence).

FIG. 18. Expression of citrus FLOWER LOCUS T gene (FT3) in tomato symbionts induces dwarfing in the host plant (Panel A) compared to tomatoes inoculated with wild-type Agrobacterium spp. only (Panel B).

FIG. 19. Citron plants infected with Candidatus Liberibacter asiaticus, the causal agent of Citrus Greening. Panels A, C and E illustrate citron with symbiont producing an antimicrobial peptide. Panels B, D and F are citron with a wild type Agrobacterium spp. as a control.

FIG. 20. Percent relative reduction in Candidatus Liberibacter asiaticus (CLas) in leaves of citrus that have 4 month-old symbionts formed on the host citrus plant by co-inoculation (see FIG. 19) and expressing the antimicrobial peptide oncocin with the oncocin operably linked to an ER targeting sequence (oncocin+), compared to citrus inoculated with a wild-type Agrobacterium spp.

FIG. 21. Graph illustrating efficacy of the expression of a product of interest against Candidatus Liberibacter asiaticus (CLas). Percent relative reduction in CLas in symbiont tissues expressing antimicrobial peptides (oncocin or TMOF) with (+) and without signal sequence compared to tissues inoculated with wildtype Agrobacterium spp. GFP+ is tissue expressing a green florescent protein with signal sequence without an antimicrobial peptide. TMOF=trypsin modulating oostatic factor.

FIG. 22. Tobacco (Nicotiana benthamiana) co-inoculated with a symbiont forming inoculum comprising a polynucleotide of interest encoding a bacterial effector protein previously shown to induce effector triggered immunity in N. benthamiana. Panel A. Pre-inoculation healthy plant. Panel B. 1-week post-inoculation, bottom arrow indicates inoculation site; Panel C. plant death 2-weeks post-inoculation.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Mention of trade names or commercial products in this publication is solely for the purpose of providing specific information and does not imply recommendation or endorsement by the U.S. Department of Agriculture.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, 5%, 1%, 0.5%, or even ±0.1% of the specified value as well as the specified value. For example, “about X” where X is the measurable value, is meant to include X as well as variations of ±10%, ±5%, 1%, 0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur, and that the description includes instances in which said event or circumstance occurs and instances where it does not. For example, the phrase “optionally comprising X” means that the composition may or may not contain X.

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control. For example, a host plant having a modified characteristic may exhibit increased tolerance or increase resistance to an insect pest, where in the increased tolerance or resistance is increased by about 5% to about 500% as compared to a control plant.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount.

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence. Thus, as used herein, the term “heterologous” refers to a nucleotide/polypeptide that originates from a foreign species, or, if from the same species, is substantially modified from its native form in composition and/or genomic locus by deliberate human intervention. As an example, a heterologous polynucleotide may encode a nucleotide sequence that is native to an organism, but which nucleotide sequence is operably linked to a heterologous promoter, thereby providing the heterologous polynucleotide.

A “native” or “wild type” nucleic acid, nucleotide sequence, polypeptide or amino acid sequence refers to a naturally occurring or endogenous nucleic acid, nucleotide sequence, polypeptide or amino acid sequence.

As used herein, the terms “nucleic acid,” “nucleic acid molecule,” “nucleotide sequence” and “polynucleotide” refer to RNA or DNA that is linear or branched, single or double-stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single-stranded or double-stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length (e.g., reduced by 1, 2, 3, 4, 5, 6, 7, 8, 9, 10, 11, 12, 13, 14, 15, 16, 17, 18, 19, 20, 20, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, or 900 or more nucleotides or any range or value therein) relative to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 260, 270, 280, 290, or more consecutive amino acids of a reference polypeptide. The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

As used herein with respect to nucleic acids, the term “functional fragment” refers to a nucleic acid that encodes a functional fragment of a polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncation of a polypeptide can be the result of a deletion of the corresponding 5′ end or 3′ end of the gene encoding the polypeptide. A frameshift mutation can occur when deletions or insertions of one or more base pairs are introduced into a gene. Frameshift mutations in a gene can result in the production of a polypeptide that is longer, shorter or the same length as the wild type polypeptide depending on when the first stop codon occurs following the mutated region of the gene. A deletion can cause a mutation in a non-coding part of the gene such as a promoter.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5). Complementarity between two single-stranded molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent sequence identity” can refer to the percentage of identical amino acids in an amino acid sequence as compared to a reference polypeptide.

As used herein, the phrase “substantially identical,” or “substantial identity” in the context of two nucleic acid molecules, nucleotide sequences, or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more in length, or any range therein, up to the full length of the sequence.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences are considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences that have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1×SSC at 45° C. for 15 minutes. An example of a low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

Any polynucleotide and/or recombinant nucleic acid molecule of this invention can be codon optimized for expression in any species of interest. Codon optimization is well known in the art and involves modification of a nucleotide sequence for codon usage bias using species specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. As is understood in the art, codon optimization of a nucleotide sequence results in a nucleotide sequence having less than 100% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original, native nucleotide sequence. Thus, in some embodiments of the invention, a polynucleotide of interest and/or a polynucleotide encoding a phytohormone biosynthetic enzyme and/or nucleic acid constructs comprising the same can be codon optimized for expression in the particular species of interest.

In some embodiments, the recombinant nucleic acid molecules, nucleotide sequences and polypeptides of the invention are “isolated.” An “isolated” nucleic acid molecule, an “isolated” nucleotide sequence or an “isolated” polypeptide is a nucleic acid molecule, nucleotide sequence or polypeptide that, by the hand of man, exists apart from its native environment and is therefore not a product of nature. An isolated nucleic acid molecule, nucleotide sequence or polypeptide may exist in a purified form that is at least partially separated from at least some of the other components of the naturally occurring organism or virus, for example, the cell or viral structural components or other polypeptides or nucleic acids commonly found associated with the polynucleotide. In some embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95%, or more pure.

In some embodiments, an isolated nucleic acid molecule, nucleotide sequence or polypeptide may exist in a non-native environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the recombinant nucleic acid construct, polynucleotides and their encoded polypeptides are “isolated” in that, by the hand of a human, they exist apart from their native environment and therefore are not products of nature, however, in some embodiments, they can be introduced into and exist in a recombinant host cell.

In any of the embodiments described herein, a polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences.

By “operably linked” or “operably associated” as used herein in reference to polynucleotides, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus and/or the C-terminus) directly (e.g., via a peptide bond) or through a linker. As an example, a polypeptide may be linked to a targeting sequence, optionally at the N-terminus or the C-terminus or both. As used herein, a “linker” may refer to a chemical group or a molecule that links two molecules or moieties.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Ann. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, Cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and Arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions. An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. An expression cassette and/or vector may comprise one or more than one polynucleotide and/or nucleic acid construct of the invention. When more than one polynucleotide and/or a nucleic acid construct is comprised in an expression cassette or vector, the more than one polynucleotide and/or a nucleic acid construct may be considered to be “stacked” in the expression cassette/nucleic acid construct. In some embodiments, a host plant may also have multiple symbionts attached that deliver expression products of the expression cassette(s) to the host plant in any combination and may also considered to be “stacked”. These could include the use of expression cassettes that have one or more polynucleotide and/or nucleic acid constructs used to generate one or more expression products to the host plant in any combination of the of stacked configuration(s).

As used herein, “expression cassette” means a recombinant nucleic acid molecule comprising a nucleotide sequence of interest (e.g., the nucleic acid constructs of the invention (e.g., a synthetic tracr nucleic acid construct, a synthetic CRISPR nucleic acid construct, a synthetic CRISPR array, a chimeric nucleic acid construct; a nucleotide sequence encoding a polypeptide of interest, a nucleotide sequence encoding a cas9 nuclease)), wherein said nucleotide sequence is operably associated with at least a control sequence (e.g., a promoter). Thus, some aspects of the invention provide expression cassettes designed to express the nucleotides sequences of the invention.

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression.

An expression cassette also can optionally include a transcriptional and/or translational termination region (i.e., termination region) that is functional in the selected host cell. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the host cell, or may be derived from another source (i.e., foreign or heterologous to the promoter, to the nucleotide sequence of interest, to the host, or any combination thereof).

An expression cassette also can include a nucleotide sequence for a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Of course, many examples of suitable selectable markers are known in the art and can be used in the expression cassettes described herein.

In addition to expression cassettes, the nucleic acid molecules and nucleotide sequences described herein can be used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Vectors for use in transformation of host organisms are well known in the art. Non-limiting examples of general classes of vectors include but are not limited to a viral vector, a plasmid vector, a phage vector, a phagemid vector, a cosmid vector, a fosmid vector, a bacteriophage, an artificial chromosome, or an Agrobacterium spp. binary vector in a double- or single-stranded linear or circular form which may or may not be self transmissible or mobilizable. A vector as defined herein can transform prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells). In some representative embodiments, the nucleic acid in the vector is under the control of, and operably linked to, an appropriate promoter or other regulatory elements for transcription in a host cell. The vector may be a bi-functional expression vector that functions in multiple hosts. In the case of genomic DNA, this may contain its own promoter or other regulatory elements and in the case of cDNA, this may be under the control of an appropriate promoter or other regulatory elements for expression in the host cell. Accordingly, the nucleic acid molecules of this invention and/or expression cassettes can be comprised in vectors as described herein and as known in the art.

As used herein, “modifying” or “modification” and grammatical variations thereof, in reference to a host plant means a change in at least one host plant characteristic without a concurrent change in the host plant genome or genotype.

The term “inoculate,” “inoculating”, “inoculated,” and grammatical variations thereof, as used herein refers to the act of contacting a biological entity (i.e. a plant) to a composition having biological activity (e.g., a symbiont forming inoculum). The composition having biological activity may be referred to as an inoculum (e.g., a symbiont forming inoculum).

As used herein, “contact”, contacting”, “contacted,” and grammatical variations thereof, refers to placing the components of a desired reaction together under conditions suitable for carrying out the desired reaction (e.g., inoculation, introducing, transformation, transfection, transplantation and the like) “Introducing,” “introduce,” “introduced” (and grammatical variations thereof) in the context of a polynucleotide (e.g., a polynucleotide encoding a phytohormone biosynthetic gene, a polynucleotide of interest) means presenting the polynucleotide to the host organism or cell of said organism (e.g., host cell) in such a manner that the polynucleotide gains access to the interior of a cell. Where more than one polynucleotide is to be introduced these polynucleotides can be assembled as part of a single polynucleotide or nucleic acid construct, or as separate polynucleotides or nucleic acid constructs, and can be located on the same or different expression constructs or transformation vectors. Accordingly, these polynucleotides can be introduced into cells in a single transformation event, in separate transformation/transfection events, or, for example, they can be incorporated into an organism by conventional breeding protocols. Thus, in some aspects of the present invention, one or more polynucleotides or nucleic acid constructs of this invention (e.g., a polynucleotide encoding a phytohormone biosynthetic enzyme and/or a polynucleotide of interest) can be introduced into a bacterial cell or a plant cell for use as a symbiont forming inoculum to generate a symbiont.

The terms “transplant” “transplanting,” or “transplantation” (and grammatical variations thereof) as used herein refers to the process of insertion into/onto at least one site on a host plant at least one plant cell (e.g., 1, 2, 3, 4, 6, 7, 8, 9, 10, 20, 30, 40, 50, 100, 200, 300, 400, 500, 1000, 5000, 10,000, 100000 or more cells) comprising one or more polynucleotides encoding at least one phytohormone biosynthetic enzyme and one or more polynucleotides of interest.

The term “transformation” or “transfection” as used herein refers to the introduction of a heterologous nucleic acid into a cell. Transformation of a cell may be stable or transient. Thus, in some embodiments, a host cell or host organism is stably transformed with a nucleic acid molecule of the invention. In other embodiments, a host cell or host organism is transiently transformed with a recombinant nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein also includes the nuclear and the plastid genome, and therefore includes integration of the nucleic acid into, for example, the chloroplast or mitochondrial genome. Stable transformation as used herein can also refer to a transgene that is maintained extrachromosomally, for example, as a minichromosome or a plasmid.

Transient transformation may be detected by, for example, an enzyme-linked immunosorbent assay (ELISA) or Western blot, or mass spectrometry, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant, a mammal, an insect, an archaea, a bacterium, and the like). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

As described herein, the polynucleotides, nucleic acid constructs, expression cassettes of this invention are stably incorporated into the genome of a symbiont or a cell of a symbiont forming inoculum.

A recombinant nucleic acid molecule/polynucleotide of the invention can be introduced into a cell by any method known to those of skill in the art. The methods of the invention do not depend on a particular method for introducing one or more nucleotide sequences into the organism, only that they gain access to the interior of at least one cell of the organism.

In some embodiments of the invention, transformation of a cell comprises nuclear transformation. In other embodiments, transformation of a cell comprises plastid transformation (e.g., chloroplast transformation.

Procedures for transforming both prokaryotic and eukaryotic organisms, including plants and bacterial cells, are well known and routine in the art and are described throughout the literature (See, for example, Jiang et al. 2013. Nat. Biotechnol. 31:233-239; Ran et al. Nature Protocols 8:2281-2308 (2013)). Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)). General guides to the transformation of yeast include Guthrie and Fink (1991) (Guide to yeast genetics and molecular biology. In Methods in Enzymology, (Academic Press, San Diego) 194:1-932) and guides to methods related to the transformation of bacteria include Aune and Aachmann (Appl. Microbiol Biotechnol 85:1301-1313 (2010)).

Where more than one polynucleotide is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, or in separate transformation events, or, alternatively, where relevant, a nucleotide sequence can be incorporated into a plant, as part of a breeding protocol.

The term “T-DNA” in the present invention refers to transfer DNA, a DNA segment in Agrobacterium species well-known in the art to be transferred to the genome of (transformed into) a plant infected by the Agrobacterium.

As used herein, the term “single-strain inoculation” refers to inoculation of a plant cell with a single bacterial strain, wherein the polynucleotide encoding a phytohormone biosynthetic enzyme and at least one polynucleotide of interest desired for transforming the plant cell are present in a single bacterial strain.

As used herein, the term “co-inoculation” refers to inoculation of a plant cell with at least two bacterial strains, wherein one strain carries the polynucleotide encoding a phytohormone biosynthetic enzyme and a separate strain carries the at least one polynucleotide of interest required for transforming a plant cell.

“Symbiont forming inoculum” as used herein refers to a composition that may be used to inoculate a host plant to produce a symbiont as described herein. In some embodiments, the “symbiont forming inoculum” may comprise a nucleic acid construct comprising a polynucleotide encoding a phytohormone biosynthetic enzyme as described herein and a polynucleotide of interest as described herein. In some embodiments, the “symbiont forming inoculum” may comprise a cell (e.g., a bacterial cell or a plant cell) comprising the polynucleotide encoding a phytohormone biosynthetic enzyme and the polynucleotide of interest. In some embodiments, a “symbiont forming inoculum” may be taken from a symbiont and may comprise a single cell or more than one cell of a symbiont (e.g., a portion of a symbiont, e.g., about 0.005 microgram to about 1 gram of a symbiont; e.g., about 0.005, 0.01, 0.05, 0.1, 0.2, 0.3, 0.4, 0.5, 1, 2, 3, 4, 5, 10, 15, 20, 25, 30, 35, 40, 45, 50, 100, 200, 300, 400, 500, 1000, 2000, 3000, 4000, 5000 micrograms to about 1, 2, 3, 4, 5, 10, 20, 30, 40, 50, 100, 200, 300, 400, 500, 600, 700, 800, 900, or 1000 milligrams, or any range or value therein).

A “polynucleotide encoding a phytohormone biosynthetic enzyme” refers to one or more than one polynucleotide (e.g., 1, 2, 3, 4, 5 or more) encoding one or more than one phytohormone biosynthetic enzyme (e.g., 1, 2, 3, 4, 5 or more), wherein the one or more than one phytohormone biosynthetic enzyme may be any cytokinin biosynthetic enzyme and/or auxin biosynthetic enzyme as described herein. In some embodiments, a phytohormone biosynthetic enzyme or a polynucleotide encoding the same may be from a bacterial species, e.g., a bacterial auxin biosynthetic enzyme or a bacterial cytokinin biosynthetic enzyme (e.g., an Agrobacterium spp. (e.g., A. tumefaciens, A. fabrum, A. rhizogenes, A. vitis), Rhizobium spp. (R. tumerigenes, R. skierniewicense, R. lusitanum), Pseudomonas savastanoi). In some embodiments, a phytohormone biosynthetic enzyme or a polynucleotide encoding the same may be from a plant species, e.g., a plant auxin biosynthetic enzyme or a plant cytokinin biosynthetic enzyme (e.g., Oryza sativa, Zea mays, Arabidopsis thaliana). In some embodiments, a phytohormone biosynthetic enzyme or a polynucleotide encoding the same may be from an insect species or may be an analog of a phytohormone. Example polynucleotides encoding a phytohormone biosynthetic enzyme include, but are not limited to, any one of the nucleotide sequences of SEQ ID NOs:1, 3, 5 or 21 or a nucleotide sequence having at least about 80% identity to the same (e.g., 80, 81, 82, 83, 84, 85, 86, 87, 88, 89, 90, 91, 92, 93, 94, 95, 96, 97, 98, 99 or 100% identity). In some embodiments, a polynucleotide encoding a phytohormone biosynthetic enzyme useful with this invention encodes any one of the amino acid sequences of SEQ ID NOs:2, 4, 6-20, 22 or 23 or an amino acid sequences having at least about 80% identity to the same (e.g., 80, 81, 82, 83, 84, 85, 86, 87, 88, 89, 90, 91, 92, 93, 94, 95, 96, 97, 98, 99 or 100% identity). Example phytohormone biosynthetic polypeptides useful with the invention includes, but are not limited to, any one of the amino acid sequences of SEQ ID NOs:2, 4, 6-20, 22 or 23 or an amino acid sequence having at least about 80% identity to the same (e.g., 80, 81, 82, 83, 84, 85, 86, 87, 88, 89, 90, 91, 92, 93, 94, 95, 96, 97, 98, 99 or 100% identity). In some embodiments, the phytohormone biosynthetic enzyme is an auxin biosynthetic enzyme. An auxin biosynthetic enzyme useful with this invention includes, but is not limited to, indole-3-acetamide hydrolase (e.g., iaaH, TMS2, AUX2) (E.C. Number: EC 3.5.1.4), amidase 1 (e.g., AtAMI1) (EC 3.5.1.4), tryptophan 2-monooxygenase (e.g., iaaM, TMS1, AUX1) (EC 1.13.12.3), indole-3-lactate synthase (EC 1.1.1.110), L-tryptophan—pyruvate aminotransferase 1 (e.g., TAA1, TIR2, CKRC1, SAV3, WEI8) (EC 2.6.1.99), tryptophan aminotransferase-related protein 1 (e.g., TAR1) (EC 2.6.1.27), indole-3-acetaldehyde oxidase (e.g., IAA oxidase, AO1, Ao-1, AtAO-1, ZmAO1, NtAO1, AtAO1) (EC 1.2.3.7), and/or tryptophan decarboxylase 1 (TDC1)/tryptophan decarboxylase 2 (TDC2) (EC4.1.1.105). In some embodiments, the phytohormone biosynthetic enzyme is a cytokinin biosynthetic enzyme. A cytokinin biosynthetic enzyme useful with this invention includes, but is not limited to, isopentenyl transferase (Ipt) (synonyms: adenosine phosphate-isopentenyltransferase; adenylate dimethylallyltransferase; (dimethylallyl)adenosine tRNA methylthiotransferase) (E.C. Number: 2.5.1.27 or 2.5.1.75 or 2.5.1.112) and/or Tzs (synonyms: dimethyl transferase, isopentenyl transferase, trans-zeatin producing protein, adenylate dimethylallyltransferase) (EC 2.5.1.27). Any combination of phytohormone biosynthetic enzymes may be used that can initiate the autonomous dividing of a plant cell to form symbiont forming inoculum and symbionts as described herein. In some embodiments, phytohormone biosynthetic enzyme combinations that may be utilized with this invention include but are not limited to, SEQ ID NO:1/2 and SEQ ID NO:3/4 and optionally, SEQ ID NO:5/6; SEQ ID NO:8 and SEQ ID NO:9; SEQ ID NO:10 and SEQ ID NO:11; and/or SEQ ID NO:12 and SEQ ID NO:13. Any combination of polynucleotides encoding auxin phytohormone biosynthetic enzymes and polynucleotides encoding cytokinin phytohormone biosynthetic enzymes that can initiate autonomous replication in a plant cell may be used to generate symbionts and symbiont forming inoculum as described herein.

A “polynucleotide of interest” refers to a polynucleotide encoding a molecule (e.g., one or more than one polypeptide, peptide, coding RNA or non-coding RNA; e.g., a bioactive molecule) for expression in a symbiont, and optionally transported from the symbiont into a host plant on which the symbiont is affixed at one or more than one site. In some embodiments, a polynucleotide of interest may encode a bioactive molecule or may encode a biosynthetic enzyme for a bioactive molecule (e.g., a polypeptide involved in the biosynthesis of a bioactive molecule).

“Modifying host plant characteristic” as used herein means altering at least one aspect or response of a host plant by growth of a symbiont of the invention on the host plant. Such aspects can include the presence of a biomolecule (produced in the symbiont and transported to the host plant) that is not otherwise found in the host plant or is found in the host plant in a reduced amount (e.g., not found in or is present in a reduced amount in the host plant not comprising the symbiont), including but not limited to, an insecticidal biomolecule, an antimicrobial biomolecule (antibacterial, antifungal), a nematicidal biomolecule, an antiviral biomolecule, an herbicidal biomolecule, a biomolecule that confers herbicide resistance/tolerance, a biomolecule that confers disease resistance/tolerance, a biomolecule that confers abiotic stress resistance/tolerance, a biomolecule that modifies plant structure and growth/morphology (e.g., nucleic acids which encode polypeptides and other factors (e.g., non-coding nucleic acids) that affect growth/morphology, phytohormones, and the like), a biostimulant, an RNA, an aptamer, and/or a pharmaceutical. In some embodiments, a “modified host plant characteristic” comprises an increased (e.g., increased by about 5, 10, 15, 20, 25, 30, 35, 40, 45, 50, 55, 60, 65, 70, 75, 80, 85, 90, 95, or 100%) amount of a biomolecule over the amount that may normally be found in the host plant. In some embodiments, a “modified host plant characteristic” includes an altered response to, for example, an insect, an herbicide, a plant pathogen (e.g., a plant pathogenic bacterium, fungus, and/or virus), a nematode, an environmental factor (e.g., heat, cold, salinity, and the like). In some embodiments, the host plant having a modified characteristic may comprise a symbiont that produces and transports to the host plant an herbicide, thereby killing the host plant. Thus, in some embodiments, a modified host plant characteristic maybe the presence of the herbicidal biomolecule and death of the host plant. In some embodiments, “modifying a host plant characteristic” can comprise modifying two or more characteristics of the host plant (e.g., 2, 3, 4, 5, 6, 7, 8, 9, 10 or more characteristics). Thus, a modified characteristic of a host plant comprising a symbiont of the invention may be the presence of two or more biomolecules (e.g., 2, 3, 4, 5, 6, 7, 8, 9, 10 or more) not otherwise present (or present at a reduced amount) in the host plant not comprising a symbiont of the invention and/or a modified characteristic of a host plant comprising a symbiont of the invention may comprise two or more altered or modified responses not otherwise observed in the host plant not comprising a symbiont of the invention. To obtain two or more modified characteristics (e.g., 2, 3, 4, 5, 6, 7, 8, 9, or 10 or more modified characteristics) in a host plant comprising a symbiont of the invention, a symbiont on a plant may comprise two or more POIs and/or a symbiont may comprise two or more symbionts (e.g., 2, 3, 4, 5, 6, 7, 8, 9, or 10 or more symbionts), wherein at least two of the two or more symbionts each comprise at least one POI that is different from a POI comprised in another symbiont.

As used herein, “symbiont” refers to a plant cell or a plurality of plant cells comprising a polynucleotide encoding a phytohormone biosynthetic enzyme (e.g., at least on polynucleotide encoding one or more phytohormone biosynthetic enzymes) and a polynucleotide of interest, wherein the one or more phytohormone biosynthetic enzymes are a cytokinin biosynthetic enzyme and/or an auxin biosynthetic enzyme, wherein the symbiont is growing on a host plant. The cell(s) of a “symbiont” autonomously divide due to the expression of the polynucleotide encoding a phytohormone biosynthetic enzyme. A “symbiont” may comprise 1, 2, 3, 4, 5, 6, 7, 8, 9, 10, 15, 20, 25, 30, 35, 40, 45, 50, 100, 150, 200, 250, 300, 350, 400, 450, 500, 600, 700, 800, 900, 1000, 1500, 2000, 2500, 3000, 3500, 4000, 4500, 5000, 6000, 7000, 8000, 9000 or 10,000, 20,000, 30,000, 40,000, 50,000, 60,000, 70,000, 80,000, 90,000 or 100,000 or more cells. Thus, in some embodiments, a symbiont may be a single plant cell that comprises at least one pSYM, a plasmid comprising at least one polynucleotide (e.g., at least 1, 2, 3, 4, 5, 6, 7, 8, 9, or 10 or more polynucleotides) encoding one or more (e.g., at least 1, 2, 3, 4, 5, 6, 7, 8, 9, or 10 or more) phytohormone biosynthetic enzymes and at least one (e.g., at least 1, 2, 3, 4, 5, 6, 7, 8, 9, or 10 or more) polynucleotide(s) of interest (POI) or it may comprise two or more cells each of which comprises at least one pSYM, a plasmid comprising at least one polynucleotide encoding one or more phytohormone biosynthetic enzymes and at least one polynucleotide of interest (POI). The cells of a symbiont autonomously divide, which form an undifferentiated multi-cellular structure on a plant. In some embodiments, the undifferentiated multi-cellular structure (e.g., symbiont) that is formed may be visually similar to, for example, a burl, a plant food body, a dormatia, an extrafloral nectary, a nodule, plant neoplasm or gall, but which are biochemically/genetically distinct by at least the transgenes expressed in the symbiont.

In some embodiments, a symbiont may be removed from the original host plant, cultured in a laboratory setting, and/or transplanted onto another plant (e.g., may be used as symbiont forming inoculum). In cases where the symbiont or at least one cell from a symbiont is cultured, “the child symbiont material” may be used to refer to the new symbiont material formed over time and propagated from the original material removed from the host plant.

The present invention is directed to a host plant comprising at least one modified characteristic without modifying the genome of the host plant. The present invention is further directed to methods and compositions for making a host plant comprising at least one modified characteristic without modifying the genome of the host plant.

The present invention takes advantage of the understanding that auxin and cytokinin genes when expressed in a plant cell can cause the plant cell to autonomously divide forming undifferentiated multicellular structures. In nature, such structures include, for example, galls that are initiated by infection of a plant by Agrobacterium spp. This ability to generate autonomously dividing cells is utilized by the present inventors along with the expression of polynucleotides of interest (POIs) in the autonomously dividing cells to generate undifferentiated multicellular structures (symbionts) that produce products through the expression of the POI(s). Understanding that such undifferentiated multicellular structures may be grown on host plants, the present inventors have now uniquely shown that the undifferentiated multicellular structures of the invention expressing POIs (symbionts of the invention) may be used to deliver products to a host plant and to modify characteristic(s) of the host plant without modifying the genome of the host plant. Such plants (e.g., host plants) and related products (symbionts and symbiont forming inoculum) are produced using various embodiments of the methods and compositions as described herein and the numerous variations and additions to the various embodiments provided herein that will be apparent to those skilled in the art in light of the instant disclosure and which do not depart from the instant invention.

In some embodiments, the present invention provides a symbiont forming inoculum, the symbiont forming inoculum comprising a polynucleotide encoding a phytohormone biosynthetic enzyme and a polynucleotide of interest, wherein the phytohormone biosynthetic enzyme is at least one cytokinin biosynthetic enzyme and/or an auxin biosynthetic enzyme.

In some embodiments, a symbiont forming inoculum may be a nucleic acid composition comprising a polynucleotide encoding a phytohormone biosynthetic enzyme and a polynucleotide of interest (e.g., a pSYM) that may be delivered to a host plant to produce a symbiont as described herein. In some embodiments, a symbiont forming inoculum may be a cell (e.g., a bacterial cell or a plant cell) comprising a polynucleotide encoding a phytohormone biosynthetic enzyme and a polynucleotide of interest (e.g., comprising a pSYM) that may be transplanted onto at least one site of a plant (e.g., a host plant) to produce a symbiont as described herein.

In some embodiments, a nucleic acid construct of this invention comprises a polynucleotide encoding a phytohormone biosynthetic enzyme and at least one polynucleotide of interest, wherein the phytohormone biosynthetic enzyme is a cytokinin biosynthetic enzyme and/or an auxin biosynthetic enzyme. As described herein, a polynucleotide encoding a phytohormone biosynthetic enzyme may encode one or more phytohormone biosynthetic enzymes. In some embodiments, the one or more phytohormone biosynthetic enzymes may be encoded by more than one polynucleotide. That is, when more than one phytohormone biosynthetic enzyme is comprised in a nucleic acid construct, it may be encoded on the same polynucleotide or on separate polynucleotides.

A phytohormone biosynthetic enzyme useful with a symbiont forming inoculum of this invention may be any auxin or cytokinin biosynthetic enzyme that can be expressed in a plant cell to produce a plant cell that autonomously divides or replicates, optionally to produce a callus culture, a suspension culture and/or an undifferentiated multi-cellular structure. In some embodiments, a phytohormone biosynthetic enzyme or polynucleotide encoding the same may be from a bacterial species, e.g., a bacterial auxin biosynthetic enzyme or a bacterial cytokinin biosynthetic enzyme. In some embodiments, a phytohormone biosynthetic enzyme or polynucleotide encoding the same may be from a plant species, e.g., a plant auxin biosynthetic enzyme or a plant cytokinin biosynthetic enzyme. Example polynucleotides encoding a phytohormone biosynthetic enzyme useful with the invention include, but are not limited to, any one of the nucleotide sequences of SEQ ID NOs:1, 3, 5 or 21 or a nucleotide sequence having at least about 80% identity to the same (e.g., 80, 81, 82, 83, 84, 85, 86, 87, 88, 89, 90, 91, 92, 93, 94, 95, 96, 97, 98, 99 or 100% identity). In some embodiments, a polynucleotide encoding a phytohormone biosynthetic enzyme useful with this invention encodes any one of the amino acid sequences of SEQ ID NOs:2, 4, 6-20, 22 or 23 or an amino acid sequences having at least about 80% identity to the same (e.g., 80, 81, 82, 83, 84, 85, 86, 87, 88, 89, 90, 91, 92, 93, 94, 95, 96, 97, 98, 99 or 100% identity). Example phytohormone biosynthetic polypeptides useful with the invention includes, but are not limited to, any one of the amino acid sequences of SEQ ID NOs:2, 4, 6-20, 22 or 23 or an amino acid sequence having at least about 80% identity to the same (e.g., 80, 81, 82, 83, 84, 85, 86, 87, 88, 89, 90, 91, 92, 93, 94, 95, 96, 97, 98, 99 or 100% identity). In some embodiments, the phytohormone biosynthetic enzyme is an auxin biosynthetic enzyme. An auxin biosynthetic enzyme useful with this invention includes, but is not limited to, indole-3-acetamide hydrolase (iaaH) (E.C. Number: EC 3.5.1.4), amidase 1 (EC 3.5.1.4), tryptophan 2-monooxygenase (IaaM) (EC 1.13.12.3), indole-3-lactate synthase (EC 1.1.1.110), L-tryptophan-pyruvate aminotransferase 1 (EC 2.6.1.99), tryptophan aminotransferase-related protein 1 (EC 2.6.1.27), indole-3-acetaldehyde oxidase (EC 1.2.3.7), and/or tryptophan decarboxylase 1/tryptophan decarboxylase 2 (EC4.1.1.105). In some embodiments, the phytohormone biosynthetic enzyme is a cytokinin biosynthetic enzyme. A cytokinin biosynthetic enzyme useful with this invention includes, but is not limited to, isopentenyl transferase (Ipt) (synonyms: adenosine phosphate-isopentenyltransferase; adenylate dimethylallyltransferase; (dimethylallyl)adenosine tRNA methylthiotransferase) (E.C. Number: 2.5.1.27 or 2.5.1.75 or 2.5.1.112) and/or Tzs (synonyms: dimethyl transferase, isopentenyl transferase, trans-zeatin producing protein, adenylate dimethylallyltransferase) (EC 2.5.1.27).

In some embodiments, a polynucleotide encoding an indole-3-acetamide hydrolase (e.g., iaaH, Aux2, Tms2) (E.C. Number: EC 3.5.1.4) includes, but is not limited to, a nucleotide sequence having at least 80% identity to SEQ ID NO:1. In some embodiments, an indole-3-acetamide hydrolase polynucleotide useful with the invention may encode an amino acid sequence having at least 80% identity to any one of SEQ ID NOs:2, 7, 9, 11, or 13. In some embodiments, an indole-3-acetamide hydrolase may comprise an amino acid sequence having at least 80% identity to any one of the amino acid sequences of SEQ ID NOs:2, 7, 9, 11, or 13. Accession Nos. (UniProt/NCBI) for further exemplary indole-3-acetamide hydrolases (and polynucleotides encoding the same) useful with embodiments of the invention include, but are not limited to, P06618, AAD30488.1, WP_010974823.1, WP_172691448.1, WP_172690897.1, WP_10891462.1, WP_172691118.1, NSZ87871.1, BAA76345.1, CAA39649.1 WP_070167543.1, P25016.1, WP_156536347.1, NSY72470.1, WP_156536347.1, WP_156638711.1, WP_045231698.1, WP_174183178.1, and/or AAB41868.1.

In some embodiments, an amidase 1 (e.g., AMI1, AtAMI1) (EC 3.5.1.4) may comprise an amino acid sequence having at least 80% identity to the amino acid sequence of SEQ ID NO:14 (At1GO8980). In some embodiments, an amidase 1 polynucleotide useful with this invention encodes an amino acid sequence having at least 80% identity to SEQ ID NO:14.

In some embodiments, a polynucleotide encoding a tryptophan 2-monooxygenase (e.g., IaaM, Tms1, Aux1) (EC 1.13.12.3) includes, but is not limited to, the nucleotide sequence of SEQ ID NO:3 or a nucleotide sequence having at least 80% identity to SEQ ID NO:3. In some embodiments, a tryptophan 2-monooxygenase polynucleotide useful with the invention may encode an amino acid sequence having at least 80% identity to any one of SEQ ID NOs:4, 8, 10, or 12. In some embodiments, a tryptophan 2-monooxygenase useful with the invention may comprise an amino acid sequence having at least 80% identity to any one of the amino acid sequences of SEQ ID NOs:4, 8, 10, or 12. Accession Nos. (UniProt/NCBI) for further exemplary tryptophan 2-monooxygenases (and polynucleotides encoding the same) include, but are not limited to, P25017, AAD30489.1, BAA76346.1, AYM09598.1, AYM14954.1, AYM61129.1 CAB44640.1, CUX71287.1, WP_040132230.1, AAF77123.1, WP_104680323.1, P25017.1, POA3V2.1, MBB3947410.1, WP_162163087.1, NSY99416.1, AKC10880.1, AVH45197.1, and/or AYD04913.1.

In some embodiments, an indole-3-lactate synthase (EC 1.1.1.110) may comprise an amino acid sequence having at least 80% identity to the amino acid sequence of SEQ ID NO:6. In some embodiments, an indole-3-lactate synthase polynucleotide useful with this invention can be the nucleotide sequence of SEQ ID NO:5 or a nucleotide sequence having at least 80% identity to SEQ ID NO:5. In some embodiments, an indole-3-lactate synthase polynucleotide encodes an amino acid sequence having at least 80% identity to SEQ ID NO:6. Accession Nos. (UniProt) for further exemplary indole-3-lactate synthases useful with this invention include, but are not limited to, WP_052675630.1, WP_083212579.1, WP_172691447.1 and/or WP_010891463.1.

In some embodiments, an L-tryptophan-pyruvate aminotransferase 1 (e.g., TAA1, TIR2, CKRC1, SAV3, WEI8) (EC 2.6.1.99) useful with the invention may comprise an amino acid sequence having at least 80% identity to the amino acid sequence of SEQ ID NO:15 (UniProt Q927N2). In some embodiments, an L-tryptophan-pyruvate aminotransferase 1 polynucleotide encodes an amino acid sequence having at least 80% identity to SEQ ID NO:15.

In some embodiments, a tryptophan aminotransferase-related protein 1 (e.g., TAR1) (EC 2.6.1.27) useful with the invention may comprise an amino acid sequence having at least 80% identity to the amino acid sequence of SEQ ID NO:16 (UniProt Q9LR29). In some embodiments, aa tryptophan aminotransferase-related protein 1 polynucleotide encodes an amino acid sequence having at least 80% identity to SEQ ID NO:16.

In some embodiments, an indole-3-acetaldehyde oxidase (e.g., IAA oxidase, AO-1, AO1, zmAO1, NtAO1, AtAO1, AtAO-1) (EC 1.2.3.7) useful with the invention may comprise an amino acid sequence having at least 80% identity to the amino acid sequence of SEQ ID NO:17 (UniProt 023887) and/or SEQ ID NO:18 (UniProt Q7G193). In some embodiments, an indole-3-acetaldehyde oxidase polynucleotide encodes an amino acid sequence having at least 80% identity to SEQ ID NO:17 and/or SEQ ID NO:18.

In some embodiments, a tryptophan decarboxylase 1 (e.g., TDC1) and/or a tryptophan decarboxylase 2 (e.g., TDC2) (EC4.1.1.105) may be used with the invention for initiating autonomous cellular division in a plant cell. A tryptophan decarboxylase 1 useful with the invention can include, but is not limited to, those comprising an amino acid sequence having at least 80% identity to the amino acid sequence of SEQ ID NO:19 (UniProt Q6ZJK7). In some embodiments, a tryptophan decarboxylase 1 polynucleotide encodes an amino acid sequence having at least 80% identity to SEQ ID NO:19. In some embodiments, a tryptophan decarboxylase 2 (e.g., TDC2) (EC4.1.1.105) can include, but is not limited to, those comprising amino acid sequence having at least 80% identity to the amino acid sequence of SEQ ID NO:20 (UniProt Q7XHL3). In some embodiments, a tryptophan decarboxylase 2 polynucleotide encodes an amino acid sequence having at least 80% identity to SEQ ID NO:20.

Cytokinin biosynthetic enzymes useful for initiating autonomous cellular division in a plant cell include, but are not limited to the cytokinin biosynthetic enzymes referred to as isopentenyl transferase (Ipt). Synonyms for Ipt enzymes include adenosine phosphate-isopentenyltransferase; adenylate dimethylallyltransferase; (dimethylallyl)adenosine tRNA methylthiotransferase) (E.C. Number: 2.5.1.27, 2.5.1.75 or 2.5.1.112). In some embodiments, a polynucleotide encoding an Ipt comprises the nucleotide sequence of SEQ ID NO:21 or a nucleotide sequence having at least 80% identity to SEQ ID NO:21. In some embodiments, an Ipt polynucleotide useful with the invention may encode an amino acid sequence having at least 80% identity to any one of SEQ ID NO:22. In some embodiments, a Ipt useful with the invention may comprise an amino acid sequence having at least 80% identity to SEQ ID NO:22. Accession Nos. (UniProt/NCBI) for further exemplary Ipt polypeptides include, but are not limited to, WP_010891460.1, NZ87873.1; WP_172690592.1; CAB44641.1, WP_172690722.1; BAA76344.1; WP_156638720.1, WP_104680324.1, NTA56762.1, WP_010892365.1, AAB41870.1; WP_032488312.1, WP_156536348.1, WP_065657522.1; WP_0324488268.1; AAZ50399.1, WP_080830665.1; AYM20353.1, WP_174005331.1, WP_173994930.1, WP_111221726.1, WP032489582.1, WP_174156215.1, WP_17404522.5.1, WP_070167542.1, WP_172691205.1 and/or CAA54540.1.

Additional cytokinin biosynthetic enzymes include adenylate dimethylallyltransferase enzymes (e.g., tzs) (EC 2.5.1.27). Synonyms for Tzs enzymes include dimethyl transferase, isopentenyl transferase, trans-zeatin producing protein, and adenylate dimethylallyltransferase. A Tzs polypeptide useful with the invention can include, but is not limited to, those comprising an amino acid sequence having at least 80% identity to the amino acid sequence of SEQ ID NO:23 (UniProt P14011). In some embodiments, a Tzs polynucleotide encodes an amino acid sequence having at least 80% identity to SEQ ID NO:23.

Any combination of auxin and cytokinin biosynthetic enzymes and/or polynucleotides encoding auxin and cytokinin biosynthetic enzymes such as those described herein may be used for the production of symbionts and/or symbiont forming inoculum. In some embodiments, the phytohormone biosynthetic enzyme encoded in a nucleic acid construct of this invention may be an indole-3-acetamide hydrolase (iaaH), a tryptophan 2-monooxygenase (IaaM), and/or an isopentenyl transferase (Ipt). In some embodiments, a nucleic acid construct of this invention may further comprise a polynucleotide encoding a phytohormone biosynthetic enzyme that is indole-3-lactate synthase.

The present inventors have shown that the expression of polynucleotides encoding phytohormone biosynthetic enzymes in plant cells as described herein can induce undifferentiated cell growth and symbiont formation on plants such as pecan, citrus, potato, tomato and Nicotiana benthamiana. It is understood from the literature related to crown gall and other similar disorders, that increased levels of cytokinins and auxins result from plant genome integration of the T-DNA containing phytohormone biosynthetic enzymes (e.g., IaaH, IaaM and Ipt) and that the elevated production of auxin and cytokinins by cells transformed with T-DNA promotes cell division. We have taken advantage of this knowledge in developing the present invention and thus production of symbionts and symbiont forming inoculum of the invention as well as host plants having a modified characteristic without a modification in its genome. Undifferentiated callus growth is the result of both elevated auxin and cytokinin levels and maintenance of a relatively high cytokinin to auxin ratio. Elevation of cytokinins and auxins are typically two-fold to over 100 times higher than that observed in non-tumorigenic tissue. For example, in tobacco cells the cytokinin to auxin ratio observed is about 40:1. In general, the ration of cytokinin to auxin ranges from about 5:1 to about 50:1 for the initiation of autonomous division and formation of an undifferentiated growth. As is known in the art, the ratio of cytokinin to auxin needed to produce undifferentiated growth can vary based on the plant species and the analytical methods used to detect different phytohormone levels.

In some embodiments, a plant cell that comprises a nucleic acid construct of this invention comprising a polynucleotide encoding a phytohormone biosynthetic enzyme, wherein the phytohormone biosynthetic enzyme is a cytokinin biosynthetic enzyme and an auxin biosynthetic enzyme, but does not comprise a polynucleotide of interest as described herein, may be referred to as an “activated cell.” Thus, an “activated cell” as used herein refers to a plant cell comprising a polynucleotide encoding a phytohormone biosynthetic enzyme, wherein the phytohormone biosynthetic enzyme is a cytokinin biosynthetic enzyme and an auxin biosynthetic enzyme which autonomously replicates. Such activated cells may be used to generate “activated tissue”. Once a polynucleotide of interest is introduced into an activated cell or the cells of activated tissue, the activated plant cell or tissue may then be referred to as symbiont forming inoculum. Symbionts are produced by transplanting a symbiont forming inoculum onto at least one site of a host plant. Since cells (one or more cells (e.g., tissue)) may be taken from symbionts for various purposes, these cells (or tissue) may be referred to as symbionts themselves or may be considered symbiont forming inoculum when transplanted onto at least one site on a host plant.

In some embodiments, a cell from a naturally formed gall, burl, a plant food body, a dormatia, an extrafloral nectary, a nodule, a plant neoplasm and/or an autonomously replicating endosperm may be used to generate a symbiont forming inoculum. Cells from such structures as these, which naturally comprise polynucleotides encoding phytohormone biosynthetic enzymes are autonomously replicating. Such cells may be used to generate symbiont forming inoculum by transforming the cell(s) with at least one POI, wherein a symbiont forming inoculum is produced that comprises cells having the polynucleotides encoding phytohormone biosynthetic enzymes and at least one POI. Similar to other symbiont forming inoculum, the symbiont forming inoculum generated in this manner may also be used to produce symbionts on host plants.

A polynucleotide of interest useful with a symbiont forming inoculum of this invention refers to a polynucleotide encoding a molecule as described herein (e.g., one or more than one polypeptide, peptide, coding RNA or non-coding RNA; e.g., a bioactive molecule) for expression in a symbiont, and optionally transported from the symbiont into a host plant on which the symbiont is affixed at one or more than one site, optionally wherein when transported into the host plant, the molecule can confer a new characteristic onto the host plant without altering the genotype or genome of the host plant. In some embodiments, a polynucleotide of interest may encode a biomolecule and/or a bioactive molecule and/or may encode a biosynthetic enzyme for a biomolecule and/or a bioactive molecule (e.g., a polypeptide involved in the biosynthesis of a biomolecule and/or bioactive molecule) as described herein. “A polynucleotide of interest” comprised in a symbiont forming inoculum may be one polynucleotide of interest or may be two or more polynucleotides of interest. When two or more polynucleotides of interest are comprised in a symbiont forming inoculum, the symbiont forming inoculum which may be referred to as a “stacked” symbiont forming inoculum. Stacked symbiont forming inoculum may be used to form one or more stacked symbionts on a host plant. As a further example of stacking, when a symbiont forming inoculum comprises bacterial cells, the bacterial cells may comprise at least two different POIs on one plasmid or at least two different plasmids.

In some embodiments, a nucleic acid construct of this invention may further comprise polynucleotide encoding a plast polypeptide (e.g., plasticity polypeptide). A plast polypeptide useful with this invention can be any plast polypeptide now known or later discovered that can confer a benefit on the morphology and structure of a symbiont that is formed using the nucleic acid constructs of this invention (see, e.g., Leon Otten, Curr Topics Microbiol Immunol 418:375-419 (2018)). Example plast polypeptides useful with nucleic acid constructs of this invention include, but are not limited to, those provided in Table 1. In some embodiments, a plast polypeptide may be a 6b, rolB, rolC, and/or orf13. In some embodiments, more than one polynucleotide encoding a plast polypeptide may be comprised in a nucleic acid construct of this invention.

In some embodiments, a polynucleotide encoding a phytohormone biosynthetic enzyme and/or a polynucleotide of interest of a symbiont forming inoculum may be operably linked to a regulatory element, including, but not limited to, a promoter sequence, a terminator sequence and/or an intron. In some embodiments, when the polynucleotide encoding a phytohormone biosynthetic enzyme and/or the polynucleotide of interest are both operably linked to a promoter, they may each be operably linked to the same promotor or separate promoters, in any combination. In some embodiments, when the polynucleotide encoding a phytohormone biosynthetic enzyme and/or the polynucleotide of interest are both operably linked to a terminator sequence, they may each be operably linked to the same terminator or separate terminators, in any combination.

A nucleic acid construct of the invention comprising a polynucleotide encoding a phytohormone biosynthetic enzyme may encode more than phytohormone biosynthetic enzyme. In some embodiments, the more than one phytohormone biosynthetic enzymes that are encoded may be operably linked to a single promoter or to separate promoters in any combination. As an example, when a polynucleotide encoding a phytohormone biosynthetic enzyme encodes a polynucleotide encoding indole-3-acetamide hydrolase (iaaH), a polynucleotide encoding tryptophan 2-monooxygenase (IaaM), and a polynucleotide encoding isopentenyl transferase (Ipt), the polynucleotide encoding iaaH, the polynucleotide encoding IaaM, the polynucleotide encoding Ipt (and/or a polynucleotide encoding indole-3-lactate synthase) and a polynucleotide of interest may each be operably linked to a single promoter or to at least two separate promoters, in any combination. In some embodiments, the polynucleotide encoding iaaH, the polynucleotide encoding IaaM, and the polynucleotide encoding Ipt (and/or a polynucleotide encoding indole-3-lactate synthase) may be operably linked to a single promoter and the at least one polynucleotide of interest may be operably linked to a separate promoter. In some embodiments, a polynucleotide encoding an indole-3-lactate synthase may be operably linked to a promoter, which may be the same promoter or a separate promoter from a promoter operably linked to any other polynucleotide encoding a phytohormone biosynthetic enzyme (e.g., a polynucleotide encoding iaaH, IaaM, and/or Ipt).

In some embodiments, a nucleic acid construct of the invention may further comprise a polynucleotide encoding a plast polypeptide, which may be operably linked to a promoter, wherein the promoter may be the same promoter or a separate promoter from the promoter operably linked to a polynucleotide operably linked to a phytohormone biosynthetic enzyme (e.g., a polynucleotide encoding iaaH, IaaM, and/or Ipt, or a polynucleotide encoding indole-3-lactate synthase). As one of skill in the art would understand any combination polynucleotides as described herein may be placed under the control of (operably linked to) one or more regulatory elements, including, but not limited to promoters and/or terminators, in any combination of separate or the same regulatory elements.

In some embodiments, a regulatory element (e.g., promoter, terminator, intron) may be endogenous to the polynucleotide to which it is operably linked or to the cell(s) of the symbiont or symbiont forming inoculum. In some embodiments, a regulatory element (e.g., promoter, terminator, intron) may be heterologous (e.g., recombinant; chimeric) to the polynucleotide to which it is operably linked or to the cell(s) of the symbiont or symbiont forming inoculum.

Any promoter functional in a plant that provides the desired expression level and location of expression in a plant cell may be used with this invention. Thus, for example, a promoter may be a constitutive promoter. In some embodiments, a promoter may be an inducible promoter. In some embodiments, a promoter that is inducible may be inducible for programmed cell death. Example promoters include but are not limited to a CaMV 35s promoter or a plant ubiquitin promoter (Ubi, e.g., Ubi-1). Additional promoters are disclosed above.

In some embodiments, for use in a symbiont forming inoculum, a polynucleotide of interest may encode a polypeptide that is operably linked to a targeting sequence so that the polypeptide may be translocated out of the symbiont into the host plant upon expression and/or located to a desired part of a host plant. Selection of the targeting sequence will depend upon the desired location of the polypeptide that is encoded by the polynucleotide of interest. In some embodiments, a targeting sequence may be used to target a protein to a membrane, a subcellular location or an extracellular location. In some embodiments, a targeting sequence is an endoplasmic reticulum targeting sequence, a mitochondrial targeting sequence, a chloroplast targeting sequence, nuclear targeting (nuclear localization) sequence, vacuolar targeting sequence, peroxisomal targeting sequence, lysosomal targeting sequence, a membrane targeting sequences, or a plant virus movement protein. In some embodiments, a polynucleotide of interest may encode a polypeptide that is operably linked to more than one (e.g., 1, 2, 3, 4, 5 or more) targeting sequence so that the polypeptide may be translocated out of the symbiont and/or located to, for example, more than one location in a host plant upon expression.

In some embodiments, a polynucleotide encoding a phytohormone biosynthetic enzyme and the polynucleotide of interest are comprised together or separately in one or more nucleic acid constructs (e.g., one or more expression cassettes) in any combination. In some embodiments, a polynucleotide encoding at least one plast polypeptide may be comprised in a nucleic acid construct, optionally wherein the polynucleotide encoding at least one plast polypeptide is comprised in the same or in a separate nucleic acid construct (e.g., expression cassette) as the polynucleotide encoding a phytohormone biosynthetic enzyme and/or the polynucleotide of interest. Nucleic acid constructs of the present invention may be comprised in or may be an expression cassette. In some embodiments, an expression cassette of the present invention may be comprised in a vector. Any vector appropriate for introducing the nucleic acid constructs into a cell may be used. As an example, a vector may include, but is not limited to, a plasmid, a T-DNA, a bacterial artificial chromosome, a viral vector, or a binary-bacterial artificial chromosome.

In some embodiments, a nucleic acid construct of the invention and/or expression cassette and/or vector comprising the same may be comprised in a cell, optionally a plant cell or a bacterial cell. Accordingly, a symbiont forming inoculum of the present invention may comprise a polynucleotide encoding a phytohormone biosynthetic enzyme and a polynucleotide of interest in a cell, wherein the phytohormone biosynthetic enzyme comprises at least one cytokinin biosynthetic enzyme and at least one auxin biosynthetic enzyme, optionally wherein the cell is a plant cell or a bacterial cell.

In some embodiments, a symbiont forming inoculum comprises a cell comprising a polynucleotide encoding a phytohormone biosynthetic enzyme and a polynucleotide of interest, wherein the cell may be a bacterial cell, optionally a bacterial cell comprising a Type IV Secretion System (T4SS, e.g., T4ASS, (e.g., VirB/D4 system), T4BSS) or a Type III Secretion System (T3SS). In some embodiments, the bacterial cell may be a cell of Agrobacterium spp., Rhizobium spp., Mesorhizobium spp., Sinorhizobium spp., Bradyrhizobium spp., Phyllobacterium spp., Ochrobactrum spp., Azobacter spp., Closterium spp., Klebsiella spp., Rhodospirillum spp., or Xanthomonas spp. In some embodiments, an Agrobacterium spp. cell may be a cell of A. tumefaciens (e.g., biovar 1), A. rhizogenes (e.g., biovar 2), A. vitis (e.g., biovar 3) or A. fabrum (e.g., strain C58). In some embodiments, a Pseudomonas spp. cell may be a cell of P. savastanoi pv. Savastanoi.

The ability of bacteria to transfer DNA to plant cells is well known both in natural settings (e.g., crown gall) and artificially (plant transformation). Researchers worldwide have taken advantage of the natural ability of Agrobacteria spp. to transfer DNA to plant cells and used this to expand well beyond the bacterium's natural host range. As one skilled in the art of plant disease and plant DNA transfer knows, the natural host range of Agrobacterium spp. is very broad. However, since at least the early 1980s, through human intervention, the ability of these bacteria to transfer DNA to plants has been extended even further to many other species that are not natural hosts. An exemplary list of plants that are natural hosts for Agrobacteria spp. and many that have been shown to be capable of being transformed using Agrobacteria spp. is provided in Table 2. These and other plant genera and species may be used as host plants or for generating symbiont forming inoculum as described herein. In some embodiments, the plant genera and species that may be used as host plants and from which symbiont forming inoculum may be made include but are not limited to those provided in Table 4 or the list of plants provided below in the paragraph prior to the examples section.

In some embodiments, a symbiont forming inoculum comprises a cell comprising a polynucleotide encoding a phytohormone biosynthetic enzyme and a polynucleotide of interest, wherein the cell may be a plant cell, optionally wherein the plant cell may be from any plant including but not limited to, an angiosperm (e.g., a dicot plant or a monocot plant), gymnosperm, an algae (e.g., a macroalgae, e.g., Rhodophyta (red algae), Phaeophyta (brown algae) and Chlorophyta (green algae), Chrysophyceae (gold algae)), a bryophyte, fern and/or fern ally (i.e., pteridophyte).

The symbiont forming inoculum comprising the polynucleotide encoding a phytohormone biosynthetic enzyme and the polynucleotide of interest, when comprised in plant cells may be in the form of a plant callus or callus culture or a suspension culture.

The present invention further provides a symbiont comprising a plant cell comprising and expressing a polynucleotide encoding a phytohormone biosynthetic enzyme and a polynucleotide of interest, wherein the phytohormone biosynthetic enzyme is at least one cytokinin biosynthetic enzyme and/or an auxin biosynthetic enzyme and the plant cell of the symbiont autonomously divides. In some embodiments, the plant cell comprises at least two plant cells (e.g., 2, 3, 4, 5, 6, 7, 8, 9, 10, 20, 30, 40, 50, 60, 70, 80, 90, 100, or more cells). A symbiont that comprises more than one cell may form a plant callus or callus culture or a suspension culture. A symbiont comprising more than one plant cell may form an undifferentiated multi-cellular structure.

A polynucleotide of interest useful with a symbiont of this invention refers to a polynucleotide encoding a molecule as described herein (e.g., one or more than one polypeptide, peptide, coding RNA or non-coding RNA; e.g., a biomolecule, a bioactive molecule) for expression in the symbiont, and optionally transported from the symbiont into a host plant on which the symbiont is affixed at one or more than one site, optionally wherein when transported into the host plant, the molecule can confer a new characteristic onto the host plant without altering the genotype or genome of the host plant. In some embodiments, a polynucleotide of interest may encode a biomolecule and/or bioactive molecule and/or may encode a biosynthetic enzyme for a biomolecule and/or bioactive molecule (e.g., a polypeptide involved in the biosynthesis of a bioactive molecule) as described herein. “A polynucleotide of interest” comprised in a symbiont may be one polynucleotide of interest or may be two or more (e.g., 2, 3, 4, 5, 6, 7, 8, 9, 10, 11, 12, 13, 14, or 15 or more) polynucleotides of interest. When two or more polynucleotides of interest are comprised in a symbiont, the symbiont may be referred to as a “stacked” symbiont. Additionally, one or more symbionts formed on a host plant, wherein at least two of the symbionts comprise a different POI, may be referred to as “stacked symbionts”. Stacking may also comprise forming one or more symbionts on a host plant, wherein all of the symbionts comprise the same POI(s).

In some embodiments, the polynucleotide encoding a phytohormone biosynthetic enzyme comprised in a symbiont may encode one or more than one phytohormone biosynthetic enzyme. In some embodiments, the one or more than one phytohormone biosynthetic enzyme may be encoded by one or more than one polynucleotide. That is, when a symbiont comprises a polynucleotide encoding more than one phytohormone biosynthetic enzyme, the more than one phytohormone biosynthetic enzyme may be encoded on the same polynucleotide as another phytohormone biosynthetic enzyme or on separate polynucleotides, in any combination.

A phytohormone biosynthetic enzyme useful with a symbiont of this invention may be any auxin or cytokinin biosynthetic enzyme that can be expressed in a plant cell to produce a plant cell that autonomously divides or replicates, optionally to produce an undifferentiated multi-cellular structure. These have been described in detail above and include auxin biosynthetic enzymes that include, but are not limited to, indole-3-acetamide hydrolase (iaaH) (E.C. Number: EC 3.5.1.4), amidase 1 (EC 3.5.1.4), tryptophan 2-monooxygenase (IaaM) (EC 1.13.12.3), indole-3-lactate synthase (EC 1.1.1.110), L-tryptophan-pyruvate aminotransferase 1 (EC 2.6.1.99), tryptophan aminotransferase-related protein 1 (EC 2.6.1.27), indole-3-acetaldehyde oxidase (EC 1.2.3.7), and/or tryptophan decarboxylase 1/tryptophan decarboxylase 2 (EC4.1.1.105). In some embodiments, the phytohormone biosynthetic enzyme is a cytokinin biosynthetic enzyme that can include, but is not limited to, isopentenyl transferase (Ipt) (synonyms: adenosine phosphate-isopentenyltransferase; adenylate dimethylallyltransferase; (dimethylallyl)adenosine tRNA methylthiotransferase) (E.C. Number: 2.5.1.27 or 2.5.1.75 or 2.5.1.112) and/or Tzs (synonyms: dimethyl transferase, isopentenyl transferase, trans-zeatin producing protein, adenylate dimethylallyltransferase) (EC 2.5.1.27). In some embodiments, the phytohormone biosynthetic enzyme may be an indole-3-acetamide hydrolase (iaaH), a tryptophan 2-monooxygenase (IaaM), and/or an isopentenyl transferase (Ipt). In some embodiments, a symbiont of this invention may further comprise a polynucleotide encoding a phytohormone biosynthetic enzyme that is indole-3-lactate synthase.

In some embodiments, a symbiont of this invention may further comprise polynucleotide encoding a plast polypeptide (e.g., plasticity polypeptide). A plast polypeptide useful with this invention can be any plast polypeptide now known or later discovered that can confer a benefit on the structure of a symbiont that is formed using the nucleic acid constructs of this invention. Example plast polypeptides useful with symbionts of this invention include, but are not limited to, those provided in Table 1. In some embodiments, a plast polypeptide may be a 6b, rolB, rolC, and/or orf13. In some embodiments, more than one polynucleotide encoding a plast polypeptide may be comprised in a symbiont of this invention.

For expression in a cell of a symbiont, a polynucleotide encoding a phytohormone biosynthetic enzyme and/or a polynucleotide of interest may be operably linked to a regulatory element, including, but not limited to, a promoter sequence, a terminator sequence and/or an intron. In some embodiments, when the polynucleotide encoding a phytohormone biosynthetic enzyme and/or the polynucleotide of interest are both operably linked to a promoter, they may each be operably linked to the same promotor or separate promoters, in any combination. In some embodiments, when the polynucleotide encoding a phytohormone biosynthetic enzyme and/or the polynucleotide of interest are both operably linked to a terminator sequence, they may each be operably linked to the same terminator or separate terminators, in any combination.

As an example, when the polynucleotide encoding a phytohormone biosynthetic enzyme encodes a polynucleotide encoding indole-3-acetamide hydrolase (iaaH), a polynucleotide encoding tryptophan 2-monooxygenase (IaaM), and a polynucleotide encoding isopentenyl transferase (Ipt), the polynucleotide encoding iaaH, the polynucleotide encoding IaaM, the polynucleotide encoding Ipt and a polynucleotide of interest may be operably linked to a single promoter or may be operably linked to at least two separate promoters, in any combination. In some embodiments, the polynucleotide encoding iaaH, the polynucleotide encoding IaaM, and the polynucleotide encoding Ipt may be operably linked to a single promoter and the at least one polynucleotide of interest may be operably linked to a separate promoter. In some embodiments, a polynucleotide encoding an indole-3-lactate synthase may be operably linked to a promoter, which may be the same promoter or a separate promoter from the promoter operably linked to any other polynucleotide operably linked to a phytohormone biosynthetic enzyme (e.g., a polynucleotide encoding iaaH, IaaM, and/or Ipt). In some embodiments, a polynucleotide encoding a plast polypeptide may be operably linked to a promoter, which may be the same promoter or a separate promoter from the promoter operably linked to a polynucleotide operably linked to a phytohormone biosynthetic enzyme (e.g., a polynucleotide encoding iaaH, IaaM, and/or Ipt, or a polynucleotide encoding indole-3-lactate synthase). As one of skill in the art would understand any combination polynucleotides as described herein may be placed under the control of (operably linked to) one or more regulatory elements, including, but not limited to promoters and/or terminators, in any combination of separate or the same regulatory elements.

In some embodiments, the regulatory element (e.g., promoter, terminator, intron) may be endogenous or heterologous (e.g., recombinant) to the polynucleotide to which it is operably linked or to the one or more plant cells of the symbiont.

Any promoter functional in a plant that provides the desired expression level and location of expression in a plant cell may be used with this invention. Thus, for example, a promoter may be a constitutive promoter. In some embodiments, a promoter may be an inducible promoter. In some embodiments, a promoter that is inducible may be inducible for programmed cell death. Example promoters include but are not limited to a CaMV 35s promoter or a plant ubiquitin promoter (Ubi, e.g., Ubi-1). Additional regulatory elements including promoters are as disclosed above.

In some embodiments, a polynucleotide encoding a phytohormone biosynthetic enzyme and the polynucleotide of interest are comprised together or separately in one or more nucleic acid constructs (e.g., one or more expression cassettes) in any combination. In some embodiments, a polynucleotide encoding at least one plast polypeptide may be comprised in a nucleic acid construct, optionally wherein the polynucleotide encoding at least one plast polypeptide is comprised in the same or in a separate nucleic acid construct (e.g., expression cassette) as the polynucleotide encoding a phytohormone biosynthetic enzyme and/or the polynucleotide of interest. Nucleic acid constructs of the present invention may be comprised in or may be an expression cassette. In some embodiments, an expression cassette of the present invention may be comprised in a vector. Any vector appropriate for introducing the nucleic acid constructs into a cell may be used. As an example, a vector may include, but is not limited to, a plasmid, a T-DNA, a bacterial artificial chromosome, a viral vector, or a binary-bacterial artificial chromosome.

In some embodiments, a polynucleotide of interest may encode a polypeptide that is operably linked to a targeting sequence so that the polypeptide may be located to a desired part of a host plant upon expression in the symbiont. Selection of the targeting sequence will depend upon the desired location of the polypeptide that is encoded by the polynucleotide of interest. In some embodiments, a targeting sequence may be used to target a protein to a membrane, a subcellular location or an extracellular location. In some embodiments, a targeting sequence is an endoplasmic reticulum targeting sequence, a mitochondrial targeting sequence, a chloroplast targeting sequence, nuclear targeting (nuclear localization) sequence, vacuolar targeting sequence, peroxisomal targeting sequence, lysosomal targeting sequence, or a plant virus movement protein. In some embodiments, a polynucleotide of interest may encode a polypeptide that is operably linked to more than one (e.g., 1, 2, 3, 4, 5 or more) targeting sequence so that the polypeptide may be located to more than one desired part of a host plant upon expression. In some embodiments, by operably linking a polypeptide to more than one targeting sequence, the polypeptide may be directed to sequentially to more than one location. For example, a polypeptide operably linked to a chloroplast targeting sequence and to a membrane targeting sequence may be first targeted to the chloroplast and then to the membrane. In some embodiments, a polynucleotide encoding a phytohormone biosynthetic enzyme (e.g., encoding iaaH, the polynucleotide encoding IaaM, and/or the polynucleotide encoding Ipt, and/or indole-3-lactate synthase) and/or a polynucleotide encoding at least one plast polypeptide may be operably linked to a nuclear targeting sequence.

In some embodiments, a symbiont comprising a polynucleotide encoding a phytohormone biosynthetic enzyme (e.g., encoding iaaH, the polynucleotide encoding IaaM, and/or the polynucleotide encoding Ipt, and/or indole-3-lactate synthase) and/or the polynucleotide encoding at least one plast polypeptide, the phytohormone biosynthetic enzyme and/or the polynucleotide encoding a plast polypeptide is/are operably linked to a nuclear targeting sequence.

In some embodiments, a symbiont may comprise a polynucleotide encoding a phytohormone biosynthetic enzyme (e.g., encoding iaaH, the polynucleotide encoding IaaM, the polynucleotide encoding Ipt) and a polynucleotide of interest, wherein the polynucleotide encoding a phytohormone biosynthetic enzyme and the polynucleotide of interest are operably linked to a single promoter or to at least two separate promoters, in any combination. In some embodiments, when the polynucleotide encoding a phytohormone biosynthetic enzyme encodes iaaH, IaaM, and Ipt, the polynucleotide(s) encoding iaaH, IaaM, and Ipt are operably linked to a single promoter and the polynucleotide of interest is operably linked to a separate promoter.

In some embodiments, a symbiont may comprise a polynucleotide encoding at least one plast polypeptide that is operably linked to a promoter, optionally wherein the polynucleotide encoding at least one plast polypeptide is operably linked to the same promoter or a separate promoter as/from the polynucleotide encoding a phytohormone biosynthetic enzyme and/or the polynucleotide of interest. In some embodiments, the promoter, the single promoter, the separate promoter and/or the two or more separate promoters are endogenous to the cells of the symbiont. In some embodiments, the promoter, the single promoter, the separate promoter and/or the two or more separate promoters are heterologous to the cells of the symbiont. In some embodiments, one or more of the promoter, the single promoter, the separate promoter and/or the two or more separate promoters may be endogenous to the cells of the symbiont, while at least one of the promoter, the single promoter, the separate promoter and/or the two or more separate promoters is heterologous to the cells of the symbiont. In some embodiments, a polynucleotide encoding a phytohormone biosynthetic enzyme may be heterologous to the plant cell of a symbiont. In some embodiments, a polynucleotide encoding a phytohormone biosynthetic enzyme may be endogenous to the plant cell of a symbiont. In some embodiments, the polynucleotide encoding a phytohormone biosynthetic enzyme may be operably linked to a heterologous promoter (e.g., heterologous to the polynucleotide encoding a phytohormone biosynthetic enzyme and/or to the plant cell of the symbiont) or to an endogenous promoter (e.g., endogenous to the polynucleotide encoding a phytohormone biosynthetic enzyme or to the plant cell symbiont).

A plant cell for use as a symbiont of this invention (e.g., a plant cell comprising a polynucleotide encoding a phytohormone biosynthetic enzyme and a polynucleotide of interest) can be any plant cell, including but not limited to, an angiosperm cell (e.g., a dicot plant or a monocot plant), gymnosperm cell, an algal cell (e.g., a macroalgae, e.g., Rhodophyta (red algae), Phaeophyta (brown algae) and Chlorophyta (green algae), Chrysophyceae (gold algae)), a bryophyte cell, fern and/or fern ally cell (i.e., pteridophyte). In some embodiments, a plant cell useful with this invention includes but is not limited to those listed in Table 2 or Table 4 or the list of plants provided below in the paragraph prior to the examples section. In some embodiments, the plant cell includes, but is not limited to, a citrus cell, a tomato cell, a corn cell, a pecan cell, and a tobacco cell.

A symbiont may be transplanted onto a plant (e.g. a host plant) at one or more locations on the plant. Accordingly, the present invention further provides a host plant comprising at least one symbiont of this invention, wherein the symbiont is located on at least one site (e.g., 1, 2, 3, 4, 5, 6, 7, 8, 9, 10, or more sites) on the plant. A plant (e.g. a host plant) of this invention may comprise more than one symbiont located on different sites of the plant or host plant. As used herein, a “site” on a plant can be any location on a plant or any plant part for growing a symbiont. Example sites or locations for a symbiont include, but are not limited to, an explant, embryo, leaf, shoot, stem, branch, kernel, ear, cob, husk, stalk, epidermal tissue, apical meristem tissue, floral tissue (e.g., pollen, pistil, ovule, anther, stamen, corolla, sepal, petal, receptacle, filament, style, stigma, etc.), fruit, seed, pod, capsule, cotyledon, hypocotyl, petiole, tuber, corm, root, root tip, symbiont, burl, plant food body, dormatia, extrafloral nectary, nodule, plant neoplasm or gall.

In some embodiments, when a symbiont is comprised on at least one site on a host plant, the polynucleotide of interest comprised in the symbiont is expressed in the symbiont and an expression product of the polynucleotide of interest and/or a product made using the expression product of the polynucleotide of interest is transported into the host plant. A host plant may be a wild type plant of any age or size (e.g., seedling, juvenile plant, or mature plant). A host plant includes, but is not limited to, an angiosperm (e.g., a dicot plant or a monocot plant), a gymnosperm, a macroalgae (e.g., Rhodophyta (red algae), Phaeophyta (brown algae) and Chlorophyta (green algae), Chrysophyceae (gold algae)), a bryophyte, and/or fern and/or fern ally (i.e., pteridophyte) as described herein. In some embodiments, a plant useful with this invention includes, but is not limited to, those listed in Table 2 and/or Table 4, and/or the list of plants provided below in the paragraph prior to the examples section. In some embodiments, example plants useful with this invention include a citrus plant (e.g., grapefruit, orange, lemon, lime, and the like), a tomato plant, a corn plant, a pecan plant, and a tobacco plant.

In some embodiments, a symbiont may be harvested from the host plant and products may be isolated/collected from the harvested symbiont, including biomolecule(s) and/or bioactive molecule(s). Any biomolecule or bioactive molecule such as those described herein may be produced in and collected/isolated from a symbiont of this invention. Product collected from symbionts and host plants comprising symbionts may be used for any purpose for which the product is suitable. Non-limiting examples of such uses include specialty chemicals, pharmaceuticals, cosmetics, lubricants, dyes/pigments, fuel, food and/or nutritional products, and the like.

The present invention further provides methods for making the compositions of this invention, including a symbiont forming inoculum, a symbiont, and a host plant comprising a symbiont of the invention. A symbiont forming inoculum of the invention can be a composition comprising one or more nucleic acid constructs (e.g., 1, 2, 3, 4, 5, or more) comprising at least one polynucleotide of interest and at least one polynucleotide encoding a phytohormone biosynthetic enzyme (e.g., encoding one or more polynucleotides (e.g., 1, 2, 3, 4, 5, or more) encoding one or more phytohormone biosynthetic enzymes (e.g., 1, 2, 3, 4, 5, or more)), wherein the biosynthetic enzyme(s) comprise(s) an auxin biosynthetic enzyme and/or a cytokinin biosynthetic enzyme. In some embodiments, a symbiont forming inoculum of the invention can comprise one or more cells (e.g., 1, 2, 3, 4, 5, 6, 7, 8, 9, 10, 15, 20, 25, 30, 35, 40, 45, 50, 100, 150, 200, 250, 300, 350, 400, 450, 500 or more cells) which comprise one or more nucleic acid constructs comprising a polynucleotide encoding phytohormone biosynthetic enzyme, wherein the biosynthetic enzyme comprises an auxin biosynthetic enzyme and/or a cytokinin biosynthetic enzyme, and a polynucleotide of interest. In some embodiments, the cell is a plant cell. In some embodiments, the cell is a bacterial cell.

Accordingly, a method of producing a symbiont forming inoculum is provided, the method comprising introducing into a cell a polynucleotide encoding a phytohormone biosynthetic enzyme and a polynucleotide of interest or introducing a polynucleotide encoding a phytohormone biosynthetic enzyme into a transgenic cell that comprises a polynucleotide of interest, wherein the phytohormone biosynthetic enzyme is at least one cytokinin biosynthetic enzyme and/or an auxin biosynthetic enzyme, thereby producing the symbiont forming inoculum. In some embodiments, the method of producing a symbiont forming inoculum further comprises culturing the cell to produce a population of cells comprising the polynucleotide encoding a phytohormone biosynthetic enzyme and the polynucleotide of interest.

The present invention further provides a method of producing a symbiont forming inoculum, the method comprising (a) (i) introducing into/onto at least one site (e.g., 1, 2, 3, 4, 5, 6, 7, 8, 9, 10 or more sites) on a plant (or a part thereof (e.g., explant, stem, and the like)) a polynucleotide encoding a phytohormone biosynthetic enzyme and a polynucleotide sequence of interest or transplanting a plant cell comprising the same (e.g., an activated plant cell comprising at least one polynucleotide encoding a phytohormone encoding enzyme) or inoculating a bacterial cell comprising the same (e.g., at least one polynucleotide encoding a phytohormone encoding enzyme) onto at least one site on the plant (or a part thereof), or (ii) introducing a polynucleotide encoding a phytohormone biosynthetic enzyme into/onto at least one site (e.g., 1, 2, 3, 4, 5, 6, 7, 8, 9, 10 or more sites) on a plant (or a part thereof (e.g., explant, stem, and the like)), the plant (or a part thereof) comprising a polynucleotide sequence of interest or transplanting a plant cell comprising the same or inoculating a bacterial cell comprising the same onto at least one site on the plant (or a part thereof), wherein the phytohormone biosynthetic enzyme is at least one cytokinin biosynthetic enzyme and/or an auxin biosynthetic enzyme, thereby producing a symbiont on the plant (or part thereof) that comprises the polynucleotide encoding a phytohormone biosynthetic enzyme and the polynucleotide sequence of interest; and (b) selecting one or more cells (e.g., 1, 2, 3, 4, 5, 6, 7, 8, 9, 10, 20, 30, 40, 50, 60, 70, 80, 90, 100, 200, 300, 400, 500, 1000, 2500, 5000, 10,000, 50,000, 100,000 or more cells; e.g., a portion, e.g., about 0.005 μg to about 1 g or more tissue from a symbiont) from the symbiont on the plant, to provide one or more cells comprising the polynucleotide encoding a phytohormone biosynthetic enzyme and the polynucleotide sequence of interest, thereby producing the symbiont forming inoculum. In some embodiments, when a method of producing a symbiont forming inoculum comprises first producing a symbiont on at least site on a plant, the at least one site on the plant is on an above ground part of the plant. In some embodiments, the at least one site on a plant is on a below ground part of the plant. In some embodiments, the method of producing a symbiont forming inoculum may further comprise (c) culturing the one or more cells from (b) to produce a population of plant cells (e.g., a callus, a callus culture and/or a suspension culture) comprising the polynucleotide encoding a phytohormone biosynthetic enzyme and the polynucleotide sequence of interest.

When a plant cell is used to produce a symbiont forming inoculum by transplanting the cell onto a plant or part thereof, or when a bacterial cell is used to produce a symbiont forming inoculum by inoculating the bacterial cell onto a plant or part thereof, the cell may be a single cell or may be two or more cells (e.g., at least 2, 3, 4, 5, 6, 7, 8, 9, 10 cells to about 100,000 cells or more). When the cell is a plant cell and the plant cell used to produce the symbiont forming inoculum comprises at least one polynucleotide encoding a phytohormone enzyme (e.g., a polynucleotide encoding an auxin biosynthetic enzyme and a polynucleotide encoding a cytokinin biosynthetic enzyme), but no polynucleotide of interest for use in modifying a host plant characteristic without modifying the host plant genome, the plant cell may be referred to an “activated” plant cell. An activated plant cell is modified with the at least one polynucleotide encoding a phytohormone enzyme allowing the cell to reproduce autonomously, thereby making the activated cell capable of forming an undifferentiated structure (a gall-like structure) when transplanted onto the plant or part thereof. When an activated plant cell autonomously divides to form tissue, the tissue can be referred to as “activated tissue.” Symbiont forming inoculum is generated from an activated plant cell or activated tissue only when the cell or tissue comprises a polynucleotide of interest for use in modifying a host plant characteristic without modifying the host plant genome.

A polynucleotide of interest useful in the methods of the invention for making compositions of this invention, including a symbiont forming inoculum, a symbiont, and a host plant comprising a symbiont of the invention includes any polynucleotide of interest that may be useful for modifying a host plant characteristic or useful in the production of a biomolecule in/by a symbiont and/or a host plant comprising at least one symbiont. A biomolecule is any molecule produced by a living organism and/or part thereof (e.g., a cell or cell free system)).

A polynucleotide of interest may encode any molecule as described herein (e.g., one or more than one polypeptide, peptide, coding RNA or non-coding RNA; e.g., a bioactive molecule), which may be expressed in a symbiont, and optionally transported from the symbiont into a host plant on which the symbiont is affixed at one or more than one site, optionally wherein when transported into the host plant, the molecule can confer a new characteristic onto the host plant without altering the genotype or genome of the host plant. In some embodiments, a polynucleotide of interest may encode a biomolecule and/or a bioactive molecule, and/or may encode a biosynthetic enzyme for a biomolecule and/or bioactive molecule (e.g., a polypeptide involved in the biosynthesis of a bioactive molecule) as described herein. As described herein, “a polynucleotide of interest” for use in making a symbiont forming inoculum as described herein may be one polynucleotide of interest or may be two or more (e.g., 2, 3, 4, 5, 6, 7, 8, 9, 10, 11, 12, 13, 14, or 15 or more) polynucleotides of interest. When two or more polynucleotides of interest are comprised in a symbiont forming inoculum, the symbiont forming inoculum may be referred to as a “stacked” symbiont forming inoculum. Stacked symbiont forming inoculum may be used to form one or more symbionts on a host plant, which may be referred to as stacked symbiont(s). As a further example of stacking, when a symbiont forming inoculum comprises bacterial cells, the bacterial cells may comprise at least two different POIs on one plasmid or at least two different plasmids.

As described herein, any auxin biosynthetic enzyme or cytokinin biosynthetic enzyme that can be expressed in a plant cell to produce a plant cell that autonomously divides or replicates as described herein may be used to make a symbiont forming inoculum. Exemplary auxin and cytokinin biosynthetic enzymes and polynucleotides encoding the same are described above in detail and include auxin biosynthetic enzymes that include, but are not limited to, indole-3-acetamide hydrolase (iaaH) (E.C. Number: EC 3.5.1.4), amidase 1 (EC 3.5.1.4), tryptophan 2-monooxygenase (IaaM) (EC 1.13.12.3), indole-3-lactate synthase (EC 1.1.1.110), L-tryptophan-pyruvate aminotransferase 1 (EC 2.6.1.99), tryptophan aminotransferase-related protein 1 (EC 2.6.1.27), indole-3-acetaldehyde oxidase (EC 1.2.3.7), and/or tryptophan decarboxylase 1/tryptophan decarboxylase 2 (EC4.1.1.105). In some embodiments, the phytohormone biosynthetic enzyme is a cytokinin biosynthetic enzyme. A cytokinin biosynthetic enzyme useful with this invention includes, but is not limited to, isopentenyl transferase (Ipt) (synonyms: adenosine phosphate-isopentenyltransferase; adenylate dimethylallyltransferase; (dimethylallyl)adenosine tRNA methylthiotransferase) (E.C. Number: 2.5.1.27 or 2.5.1.75 or 2.5.1.112) and/or Tzs (synonyms: dimethyl transferase, isopentenyl transferase, trans-zeatin producing protein, adenylate dimethylallyltransferase) (EC 2.5.1.27). In some embodiments, the phytohormone biosynthetic enzyme may be an indole-3-acetamide hydrolase (iaaH), a tryptophan 2-monooxygenase (IaaM), and/or an isopentenyl transferase (Ipt) and may optionally include indole-3-lactate synthase, and any combination thereof.

In some embodiments, a method of producing a symbiont forming inoculum may further comprise introducing into a cell or at least one site on a plant a polynucleotide encoding at least one a plast polypeptide (e.g., a plasticity polypeptide), optionally wherein the plast polypeptide includes, but is not limited to, the plast polypeptides provided in Table 1. In some embodiments, the plast polypeptide is 6b, rolB, rolC, and/or orf13.

A polynucleotide encoding a phytohormone biosynthetic enzyme and a polynucleotide of interest for introducing into a cell may be comprised together or separately in one or more nucleic acid constructs (e.g., one or more expression cassettes and/or vectors) (e.g., 1, 2, 3, 4, 5, 6, 7, 8, 9, 10, or more constructs). In some embodiments, a polynucleotide encoding a plast polypeptide (e.g., at least one plast polypeptide, e.g., 1, 2, 3, 4, 5, 6 or more) may be comprised in one or more nucleic acid constructs (e.g., 1, 2, 3, 4, 5, 6, 7, 8, 9, 10, or more constructs), optionally wherein the polynucleotide encoding at least one plast polypeptide is in the same or a separate nucleic acid construct (e.g., expression cassette) as the polynucleotide encoding a phytohormone biosynthetic enzyme and/or the polynucleotide of interest. In some embodiments, a nucleic acid construct comprising a polynucleotide encoding a phytohormone biosynthetic enzyme, a polynucleotide of interest and/or a polynucleotide encoding a plast polypeptide may be comprised in expression cassettes, which may be the same or separate expression cassettes. In some embodiments, the one or more nucleic acid constructs (or expression cassettes comprising the same) may be comprised in one or more vectors (e.g., 1, 2, 3, 4, 5, 6, 7, 8 or more). Any vector useful for transferring polynucleotides to a cell may be used with the nucleic acid constructs of the invention. In some embodiments, a vector may be a plasmid, a T-DNA, a bacterial artificial chromosome, viral vector or a binary-bacterial artificial chromosome, or any combination thereof for use with the polynucleotides, nucleic acid constructs, and/or expression cassettes of the invention.

In some embodiments, a polynucleotide of interest introduced into a cell according to the methods of this invention can encode a polypeptide operably linked to a targeting sequence. In some embodiments, the targeting sequence locates the protein to a membrane, a subcellular location or an extracellular location. In some embodiments, the targeting sequence can be, but is not limited to, a membrane targeting sequence, an endoplasmic reticulum targeting sequence, a mitochondrial targeting sequence, a chloroplast targeting sequence, or a plant virus movement protein.

In some embodiments, a polynucleotide may be targeted to the nucleus. Thus, a polynucleotide encoding a phytohormone biosynthetic enzyme as described herein and/or a polynucleotide encoding a plast polypeptide as described herein may be operably linked to a nuclear localization sequence for targeting to the nucleus of a cell.

In some embodiments, a polynucleotide encoding a phytohormone biosynthetic gene and/or a polynucleotide of interest may be operably linked to a regulatory element, including, but not limited to, a promoter sequence, a terminator sequence and/or an intron. In some embodiments, when the polynucleotide encoding a phytohormone biosynthetic gene and/or the polynucleotide of interest are both operably linked to a promotor, each may be operably linked to the same promotor or separate promoters, in any combination. In some embodiments, when the polynucleotide encoding a phytohormone biosynthetic gene and/or the polynucleotide of interest are both operably linked to a terminator sequence, each may be operably linked to the same terminator or separate terminators, in any combination. In some embodiments, a polynucleotide encoding a phytohormone biosynthetic enzyme and a polynucleotide of interest are each operably linked to a single promoter. In some embodiments, a polynucleotide encoding a phytohormone biosynthetic enzyme and a polynucleotide of interest are operably linked to at least two separate promoters, in any combination. In some embodiments, when the polynucleotide encoding a phytohormone biosynthetic enzyme encodes two or more phytohormone biosynthetic enzymes (e.g., iaaH, IaaM, and Ipt), the polynucleotide(s) encoding two or more phytohormone biosynthetic enzymes are operably linked to a single promoter and the polynucleotide of interest is operably linked to a separate promoter.

In some embodiments, when more than one polynucleotide encoding a phytohormone biosynthetic enzyme is introduced (e.g., a polynucleotide encoding iaaH, a polynucleotide encoding IaaM, a polynucleotide encoding Ipt, and/or a polynucleotide encoding an indole-3-lactate synthase), the more than one polynucleotide encoding a phytohormone biosynthetic enzyme may be operably linked to the same or to separate promoters, which may be the same or a separate promoter from a promoter operably linked to the polynucleotide of interest. In some embodiments, a polynucleotide encoding iaaH, a polynucleotide encoding IaaM, and a polynucleotide encoding Ipt are operably linked to a single promoter and the polynucleotide of interest is operably linked to a separate promoter. In some embodiments, the polynucleotide encoding a phytohormone biosynthetic enzyme (e.g., iaaH, IaaM, and Ipt and/or indole-3-lactate synthase) is operably linked to a single promoter and the polynucleotide of interest is operably linked to the same promoter.

In some embodiments, a polynucleotide encoding at least one plast polypeptide may be operably linked to a promoter. In some embodiments, a polynucleotide encoding at least one plast polypeptide is operably linked to the same promoter as that which is operably linked to the polynucleotide encoding a phytohormone biosynthetic enzyme and/or the polynucleotide of interest. In some embodiments, a polynucleotide encoding at least one plast polypeptide is operably linked to a separate promoter from the promoter that is operably linked to the polynucleotide encoding a phytohormone biosynthetic enzyme and/or the polynucleotide of interest.

Any promoter that allows the polynucleotide encoding a phytohormone enzyme and/or a polynucleotide of interest to be expressed may be used. As described herein, the selection of a promoter may vary depending on the temporal and spatial requirements for expression, and also may vary based on the host cell to be transformed. Promoters for many different organisms and having different expression patterns are well known in the art. A promoter useful in making a symbiont forming inoculum may be endogenous to the one or more cells of a symbiont forming inoculum or may be heterologous to the one or more cells of a symbiont forming inoculum, or any combination thereof. In some embodiments, the promoter may be endogenous or may be heterologous to the polynucleotide to which the promoter is operably linked

In some embodiments, a promoter useful in the making of a symbiont forming inoculum is a constitutive promoter. In some embodiments, a promoter useful in the making of a symbiont forming inoculum is an inducible promoter.

A bacterial cell useful for producing a symbiont forming inoculum may be any bacterial cell comprising a Type IV Secretion System (T4SS, e.g., T4ASS (e.g., VirB/D4 system), (T4BSS) or a Type III Secretion System (T3SS). Such bacterial systems are well known in the art and include, but are not limited to, those of Agrobacterium spp. (e.g., A. tumefaciens (e.g., biovar 1), A. rhizogenes (e.g., biovar 2), A. vitis (e.g., biovar 3), A. fabrum (e.g., strain C58), Rhizobium spp., Mesorhizobium spp., Sinorhizobium spp., Bradyrhizobium spp., Pseudomonas spp. (e.g., P. savastanoi pv. Savastanoi), Phyllobacterium spp., Ochrobactrum spp., Azobacter spp., Closterium spp., Klebsiella spp., Rhodospirillum spp., orXanthomonas spp.

Any plant cell that then may be used to form a symbiont on a plant may be used for producing symbiont forming inoculum. Such plant cells include, but are not limited to, those from an angiosperm (e.g., a dicot plant or a monocot plant), gymnosperm, an algae (e.g., a macroalgae, e.g., Rhodophyta (red algae), Phaeophyta (brown algae) and Chlorophyta (green algae), Chrysophyceae (gold algae)), a bryophyte, fern and/or fern ally (i.e., pteridophyte). The cell may be from a wild type plant or a transgenic plant of any age or size (e.g., seedling, juvenile plant, or mature plant). In some embodiments, a plant cell useful with this invention includes, but is not limited to, those listed in Table 2, Table 4 or the list of plants provided below in the paragraph prior to the examples section. In some embodiments, example plant cells useful with this invention include a citrus cell (e.g., grapefruit, orange, lemon, lime and the like), a tomato cell, a corn cell, a pecan cell, and a tobacco cell.

A plant cell useful for producing a symbiont forming inoculum can be from any plant part, including but not limited to, a plant cell culture (callus, callus culture or suspension culture), a protoplast, seedling, explant, embryo, leaf, shoot, stem, branch, kernel, ear, cob, husk, stalk, epidermal tissue, apical meristem tissue, floral tissue (e.g., pollen, pistil, ovule, anther, stamen, corolla, sepal, petal, receptacle, filament, style, stigma, etc.), fruit, seed, pod, capsule, cotyledon, hypocotyl, petiole, tuber, corm, root, root tip, symbiont, burl, plant food body, dormatia, extrafloral nectary, nodule, gall or plant neoplasm.

As described herein, in some embodiments, when producing a symbiont forming inoculum using plant cells, the at least one site on a plant can be any site on the plant including, but not limited to, an explant, embryo, leaf, shoot, stem, branch, kernel, ear, cob, husk, stalk, epidermal tissue, apical meristem tissue, floral tissue (e.g., pollen, pistil, ovule, anther, stamen, corolla, sepal, petal, receptacle, filament, style, stigma, etc.), fruit, seed, pod, capsule, cotyledon, hypocotyl, petiole, tuber, corm, root, root tip, symbiont, burl, plant food body, dormatia, extrafloral nectary, nodule, plant neoplasm or gall.

A nucleic acid construct (e.g., a polynucleotide, an expression cassette and/or a vector) may be introduced into a cell via any method known method. Procedures for transforming both prokaryotic and eukaryotic organisms, including plants, are well known and routine in the art and are described throughout the literature. In some embodiments nucleic acid construct of this invention (e.g., a polynucleotide encoding a phytohormone biosynthetic enzyme, a polynucleotide of interest and/or an expression cassette and/or a vector comprising the same) may be introduced into a cell via a method including but not limited to bacterial mediated transformation, agroinfiltration, viral-mediated transformation, particle bombardment (biolistics), electroporation, microinjection, lipofection (liposome mediated transformation), sonication, silicon fiber mediated transformation, chemically stimulated DNA uptake (e.g., polyfection; e.g., polyethylene glycol (PEG) mediated transformation), and/or laser microbeam (UV) induced transformation.

In some embodiments, when (i) the polynucleotide encoding a phytohormone biosynthetic enzyme and a polynucleotide sequence of interest or (ii) the polynucleotide encoding a phytohormone biosynthetic enzyme are comprised in at least one plant cell, the at least one plant cell may be transplanted onto at least one site (e.g., 1, 2, 3, 4, 5, 6, 7, 8, 9, 10 or more sites) on the plant. In some embodiments, the one or more cells (e.g., 1, 2, 3, 4, 5, 6, 7, 8, 9, 10, 20, 30, 40, 50, 60, 70, 80, 90, 100 or more cells) transplanted at the at least one site are cultured at the site to produce a population of plant cells comprising the polynucleotide encoding a phytohormone biosynthetic enzyme and the polynucleotide sequence of interest and form a symbiont, wherein one or more cells from the symbiont on the plant are selected to provide one or more cells comprising the polynucleotide encoding a phytohormone biosynthetic enzyme and the polynucleotide sequence of interest, thereby producing the symbiont forming inoculum.

In some embodiments, when producing a symbiont forming inoculum using a plant, the at least one site on the plant may be wounded at the site of inoculation prior to, concurrently with, or after the step of introducing at the at least one site (e.g., 1, 2, 3, 4, 5, 6, 7, 8, 9, 10 or more sites) on the plant. Similarly, when a symbiont is transplanted onto at least one site on a host plant, the at least one site on the host plant may be wounded prior to, concurrently with, or after the step of transplanting. Wounding for introducing or transplanting can be carried out in any manner that results in a breaking in the outer surface (epidermis, cuticle, bark) of the plant or part thereof at the site at which the introducing or transplanting is to occur. Such tools can include, but are not limited to, a tweezer or forceps, a knife, a needle e.g., (e.g., hypodermic, dissecting, tattoo, sewing, and the like), a toothpick, and/or a syringe. In addition, any standard grafting tools may be utilized for introducing or transplanting as described herein.

In some embodiments, introducing a polynucleotide of the invention (e.g., a polynucleotide encoding a phytohormone biosynthetic enzyme (e.g., at least one polynucleotide encoding at least one phytohormone biosynthetic enzyme), a polynucleotide of interest; expression cassette(s) or vector(s) comprising the same) into a plant cell, plant or part thereof is carried out via bacterial mediated transformation and comprises co-cultivating the plant cell or plant (or a part thereof, e.g., explant) with the cells of at least one bacterial species or strain (e.g., 1, 2, 3, 4, 5, or more), the bacterial cells comprising one or more of: the polynucleotide encoding a phytohormone biosynthetic enzyme, the polynucleotide of interest, and/or at least one polynucleotide encoding at least one plast polypeptide. In some embodiments, the plant (or part thereof; e.g., explant) may be wounded at the site of inoculation prior to or during co-cultivation with the cells of the at least one bacterial strain. In some embodiments, the cells of the at least one bacterial species or strain comprise cells of at least two bacterial species or strains and the polynucleotide encoding a phytohormone enzyme is comprised in a separate bacterial strain from the bacterial strain comprising the at least one polynucleotide of interest (e.g., dual bacterial transformation). As described herein, a bacterial cell useful for producing a symbiont forming inoculum may be any bacterial cell comprising a Type IV Secretion System (T4SS, e.g., T4ASS, (e.g., VirB/D4 system), T4BSS) or a Type III Secretion System (T3SS), and can include, but are not limited to, those of Agrobacterium spp. (e.g., A. tumefaciens (e.g., biovar 1), A. rhizogenes (e.g., biovar 2), A. vitis (e.g., biovar 3), A. fabrum (e.g., strain C58), Rhizobium spp., Mesorhizobium spp., Sinorhizobium spp., Bradyrhizobium spp., Pseudomonas spp., Phyllobacterium spp., Ochrobactrum spp., Azobacter spp., Closterium spp., Klebsiella spp., Rhodospirillum spp., or Xanthomonas spp. In some embodiments, a Pseudomonas spp. (e.g., P. savastanoi pv. Savastanoi). In some embodiments, a bacterial cell may be a Pseudomonas savastanoi pv. Savastanoi cell. The plant species to which this method may be applied is not limited. As discussed above, since at least the early 1980's, through human intervention, the ability of bacteria to transfer DNA to plants has been extended to many species, beyond those that are naturally infected by the bacteria. Non-limiting examples of plants that are natural hosts for Agrobacteria spp. and some plants that are not natural hosts but which have been shown to be capable of being transformed using Agrobacteria spp. are provided in Table 2. As would be readily understood by those of skill in the art, the plant genera and species set forth Table 2, as well as any other plant genera and species, may be used as host plants or for generating symbiont forming inoculum as described herein. In some embodiments, the plant genera and species that may be used as host plants and from which symbiont forming inoculum may be made include, but are not limited to, those provided in Table 4 or the list of plants provided below in the paragraph prior to the examples section.

In some embodiments, a method for producing a symbiont forming inoculum may further comprise editing at least one nucleic acid in at least one cell of the symbiont forming inoculum to produce at least one edited nucleic acid in the symbiont forming inoculum. Any known gene editing technology may be used including, but not limited to, a nuclease based editing system including but not limited to CRISPR-Cas technology, zinc finger nuclease (ZFN) technology; Transcription Activator-Like Effector Nuclease (TALEN) technology and engineered meganucleases technology. In some embodiments, the at least one edited nucleic acid has modified expression. In some embodiments, modified expression comprises increased expression as compared to the same nucleic acid that does not comprise the same modification. In some embodiments, modified expression comprises decreased expression as compared to the same nucleic acid that does not comprise the same modification.

The present invention further provides a symbiont forming inoculum produced by the methods of the invention. In some embodiments, the symbiont forming inoculum is a bacterial culture comprising polynucleotide encoding a phytohormone biosynthetic enzyme (e.g., one or more (e.g., 1, 2, 3, 4, 5, or more) polynucleotides encoding one or more (e.g., 1, 2, 3, 4, 5, or more) phytohormone biosynthetic enzymes) and a polynucleotide of interest (e.g., at least one polynucleotide of interest (e.g., 1, 2, 3, 4, 5, or more)). In some embodiments, the symbiont forming inoculum comprises two or more cells is in the form of a plant cell culture (e.g., a callus or a cell suspension) comprising polynucleotide encoding a phytohormone biosynthetic enzyme (e.g., one or more polynucleotides encoding one or more phytohormone biosynthetic enzymes) and a polynucleotide of interest (e.g., at least one polynucleotide of interest).

In some embodiments, the present invention provides a cell or protoplast from the symbiont forming inoculum of the invention, wherein the cell or protoplast comprises the polynucleotide encoding a phytohormone biosynthetic enzyme and the polynucleotide of interest.

Further provided is a method of modifying a host plant characteristic without modifying the plant genome, the method comprising transplanting a symbiont forming inoculum of the present invention or a symbiont of the present invention to at least one site (e.g., 1, 2, 3, 4, 5, 6, 7, 8, 9, 10 or more sites) on a host plant; and culturing the symbiont forming inoculum at the at least one site on the host plant to form a symbiont on the host plant at the at least one site, wherein the polynucleotide of interest is expressed in the symbiont on the host plant and an expression product of the polynucleotide of interest and/or a product made using the expression product of the polynucleotide of interest is transported into the host plant, thereby modifying the host plant characteristic. “Modifying a host plant characteristic without modifying the plant genome” refers to a change in the morphology, metabolism, biochemistry, and/or physiology of the host plant without changing the genotype of the host plant.

A polynucleotide of interest useful with a symbiont of this invention for modifying a plant host characteristic can include a polynucleotide encoding a molecule as described herein (e.g., one or more than one polypeptide, peptide, coding RNA or non-coding RNA; e.g., a biomolecule, a bioactive molecule) for expression in a symbiont affixed at one or more than one site on the host plant, which molecule when transported into the host plant, the molecule can confer a new characteristic onto the host plant without altering the genotype or genome of the host plant. In some embodiments, a polynucleotide of interest may encode a biomolecule or a bioactive molecule or may encode a biosynthetic enzyme for a biomolecule and/or bioactive molecule (e.g., a polypeptide involved in the biosynthesis of a biomolecule or a bioactive molecule) as described herein. As described herein, “a polynucleotide of interest” comprised in a symbiont formed on a host plant may be one polynucleotide of interest or may be two or more (e.g., 2, 3, 4, 5, 6, 7, 8, 9, 10, 11, 12, 13, 14, or 15 or more) polynucleotides of interest. When two or more polynucleotides of interest are comprised in a symbiont, the symbiont may be referred to as a “stacked” symbiont. Additionally, one or more symbionts formed on a host plant, wherein at least two of the symbionts comprise a different POI, may be referred to as “stacked symbionts”. Stacking may also comprise forming one or more symbionts on a host plant, wherein all of the symbionts comprise the same POI(s).

In some embodiments, the polynucleotide encoding a phytohormone biosynthetic enzyme comprised in a symbiont used to confer a modified host plant characteristic may encode one or more than one phytohormone biosynthetic enzyme. In some embodiments, the one or more than one phytohormone biosynthetic enzyme may be encoded by one or more than one polynucleotide. That is, when a symbiont comprises a polynucleotide encoding more than one phytohormone biosynthetic enzyme, the more than one phytohormone biosynthetic enzyme may be encoded on the same polynucleotide as another phytohormone biosynthetic enzyme or on separate polynucleotides, in any combination.

A phytohormone biosynthetic enzyme to be expressed in a symbiont of this invention may be any auxin or cytokinin biosynthetic enzyme that can be expressed in a plant cell to produce a plant cell that autonomously divides or replicates, optionally to produce an undifferentiated multi-cellular structure. As described herein, any auxin biosynthetic enzyme or cytokinin biosynthetic enzyme that can be expressed in a plant cell to produce a plant cell that autonomously divides or replicates as described herein may be used to make a symbiont forming inoculum. Exemplary auxin and cytokinin biosynthetic enzymes and polynucleotides encoding the same are described above in detail and include auxin biosynthetic enzymes that include, but are not limited to, indole-3-acetamide hydrolase (iaaH) (E.C. Number: EC 3.5.1.4), amidase 1 (EC 3.5.1.4), tryptophan 2-monooxygenase (IaaM) (EC 1.13.12.3), indole-3-lactate synthase (EC 1.1.1.110), L-tryptophan-pyruvate aminotransferase 1 (EC 2.6.1.99), tryptophan aminotransferase-related protein 1 (EC 2.6.1.27), indole-3-acetaldehyde oxidase (EC 1.2.3.7), and/or tryptophan decarboxylase 1/tryptophan decarboxylase 2 (EC4.1.1.105). In some embodiments, the phytohormone biosynthetic enzyme is a cytokinin biosynthetic enzyme. A cytokinin biosynthetic enzyme useful with this invention includes, but is not limited to, isopentenyl transferase (Ipt) (synonyms: adenosine phosphate-isopentenyltransferase; adenylate dimethylallyltransferase; (dimethylallyl)adenosine tRNA methylthiotransferase) (E.C. Number: 2.5.1.27 or 2.5.1.75 or 2.5.1.112) and/or Tzs (synonyms: dimethyl transferase, isopentenyl transferase, trans-zeatin producing protein, adenylate dimethylallyltransferase) (EC 2.5.1.27). In some embodiments, the phytohormone biosynthetic enzyme may be an indole-3-acetamide hydrolase (iaaH), a tryptophan 2-monooxygenase (IaaM), and/or an isopentenyl transferase (Ipt) and may optionally include indole-3-lactate synthase, and any combination thereof. In some embodiments, the phytohormone biosynthetic enzyme may be an indole-3-acetamide hydrolase (iaaH), a tryptophan 2-monooxygenase (IaaM), and/or an isopentenyl transferase (Ipt), in any combination. In some embodiments, a symbiont of this invention may further comprise a polynucleotide encoding a phytohormone biosynthetic enzyme that is indole-3-lactate synthase.

In some embodiments, a symbiont of this invention may further comprise and express a polynucleotide encoding a plast polypeptide (e.g., plasticity polypeptide). A plast polypeptide useful with this invention can be any plast polypeptide now known or later discovered that can confer a benefit on the structure of a symbiont that is formed using the nucleic acid constructs of this invention. Example plast polypeptides useful for symbionts of this invention, include but are not limited to those, provided in Table 1. In some embodiments, a plast polypeptide may be a 6b, rolB, rolC, and/or orf13. In some embodiments, more than one polynucleotide encoding a plast polypeptide may be comprised in a symbiont of this invention.

In some embodiments, culturing a symbiont forming inoculum, when comprised in a bacterial cell on a host plant, can further comprise culturing in the presence of acetosyringone at a concentration in a range from about 10 μM to about 200 μM or any range or value therein (e.g., about 10, 15, 20, 25, 30, 350 40, 45, 50, 60, 70, 80, 90, 100, 110, 120, 130, 140, or 150 μM, or any range or value therein)(e.g., about 50 μM-about 150 μM, about 75 μM to about 125 μM, about 85 μM to about 100 μM). In some embodiments, when culturing in the presence of acetosyringone, the acetosyringone is present at a concentration of about 100 μM.

In some embodiments, a symbiont forming inoculum comprising bacterial cells may be used to modify a host plant characteristic without modifying the host plant genome. In some embodiments, a symbiont forming inoculum containing Agrobacterium spp. may be delivered, for example, to a first plant. The Agrobacterium spp. may be in the form of one or multiple strains, where at least one strain contains a nucleic acid encoding at least one phytohormone biosynthetic enzyme (that may be provided, for example, in a T-DNA) that induces symbiont-formation and at least one strain contains a nucleic acid that comprises a polynucleotide of interest (that may be provided, for example, in a T-DNA) encoding a desired trait to be imparted to a host plant. The delivery of the inoculum may thus cause one or more (e.g., 1, 2, 3, 4, 5, 6, 7, 8, 9, 10, 12, 13, 14, 15, 16, 17, 18, 19, 20 or more) symbionts to form on the first plant, and the symbionts may express the nucleic acids delivered by the Agrobacterium spp. The symbionts have increased vascularization in the symbiont tissue, which itself supports rapid growth, more rapid metabolism, and an effective pathway for export and ultimately systemic movement of desired molecules throughout the plant. In some embodiments, a symbiont may then be removed from the first plant and affixed/transplanted onto a second plant (e.g., a host plant) so as to be in functional communication with the host plant, thus forming a plant tissue which supplies the host plant with the desired trait but without transforming or altering the genome of the host plant or introducing heterogeneous or xenobiotic DNA into the host plant. In some embodiments, prior to transplantation to the host plant, the removed symbiont, now symbiont forming inoculum may be cultured without Agrobacterium spp. to form a bacteria-free symbiont forming inoculum after which the symbiont forming inoculum may be transplanted to the host plant.

Regarding the choice of Agrobacterium spp. strain(s) to be used in the present invention, various single strains or combinations thereof are usable to achieve the desired results. According to one embodiment, the inoculum includes at least two strains where at least one strain used is an “activated strain” (such as a wild-type strain) that comprises at least one polynucleotide encoding a phytohormone biosynthetic enzyme, and at least one other strain is not an activated strain (e.g., “disarmed”, “trait inducing” strains) but comprises nucleic acid (e.g., T-DNA) that imparts a desired trait (polynucleotide of interest) in the host plant. The activated strain may be isolated from nature, such as the FL-F54 strain described herein, as wild-type Agrobacterium spp. are known to form galls. The desired trait may be, for example, having antimicrobial or anti-insect properties, a change in plant physiology, or others. The trait may be expressed or effected by one or more molecules (e.g., 1, 2, 3, 4, 5, 6, 7, 8, 9, 10 or more molecules), such as molecules encoded by the nucleic acid (e.g., T-DNA) in the trait-inducing Agrobacterium spp. These molecules may be small molecules, large molecules, proteins, polymers, or other, as desired. Multiple activated strains and/or multiple trait-inducing strains may be used, as desired for a particular application. Alternatively, a single strain may be used that both induces symbiont formation and also induces a desired trait in a host plant to which the symbiont is affixed without modifying the host plant genome. The plant species to which this method may be applied is not limited. Today, it is routine to transfer DNA to plants using Agrobacteria and other bacterial spp. Non-limiting examples of plants that are natural hosts for Agrobacteria spp. and some plants that are not natural hosts, but which have been shown to be capable of being transformed using Agrobacteria spp., are provided in Table 2. The plants listed in this table are from many different plant families, including both dicots and monocots, and demonstrate that the types of plants with which this method may be used are not limited. In some embodiments, the plant genera and species that may be used with this method include, but are not limited to, those provided in Table 4 or the list of plants provided below in the paragraph prior to the examples section.

The inoculum may contain one or more Agrobacterium spp. strain(s) (e.g., 1, 2, 3, 4, 5, or more strains) as described above in addition to a carrier, and other ingredients, as desired. If multiple strains are used, various ratios of strains may be used, as desired, for example, a 1:10 ratio of activated strain to trait-inducing strain. Agrobacterium spp. delivery inoculums are well-known in the art, and a suitable one can be chosen based on the desired outcome in a particular application. For example, the inoculum may contain an aqueous solution of a buffer, such as MES (2-ethanesulfonic acid), Tris (tris(hydroxymethyl)aminomethane), HEPES (4-(2-hydroxyethyl)-1-piperazineethanesulfonic acid), or a salt-based buffer such as PBS (phosphate-buffered saline); one or more salts, such as magnesium chloride, a transformation enhancer, such as acetosyringone or other phenolics that can enhance virulence and/or an adjuvant including, but not limited to, wetting/penetrating enhancing surfactant agents, including but not limited to anionic, cationic, and nonionic surfactants. The delivery of the inoculum may be achieved by any known method, such as via a needle, a puncture wound, or other direct delivery systems, i.e. use of drilling or air blasting, and may be automated or manual.

Symbiont formation can be observed by eye, and symbiont size can optionally be controlled via known means, such as chemical control (i.e. GALLEX® (AgBioChem Inc., Los Molinos, Calif.)). Symbiont formation may take various amounts of time, depending on the host plant species and the age of the plant used. For example, sufficient symbiont formation may take several days to several months to develop. In some embodiments, a symbiont or symbiont tissue can be collected from a first plant and then cultured for increased volume or storage purposes. In some embodiments, a symbiont may be moved directly from a first plant to a second plant (e.g., host plant) without culturing. However, it may be desired to culture the symbiont forming inoculum first to (a) remove residual bacteria, such as by attrition or by active sterilization, or (b) determine that the symbiont forming inoculum expresses the desired trait(s). Removal of residual Agrobacterium spp. may occur over time by attrition, such as by supplying a culture that does not support the bacteria and thus it dies off, or by active means, such as by sterilization with the application of bleach and/or antibiotics or other methods which actively kill bacterial cultures. The determination of whether the symbiont informing inoculum or a symbiont expresses a desired trait may be accomplished by simple observation, if the trait is phenotypically visible (such as a color), or by analysis of the culture medium/host plant for the target compound(s) being produced by the symbiont or symbiont forming inoculum, or by any other known means.

A symbiont may be removed from a plant and use as symbiont forming inoculum to affix (transplant) to a second plant (e.g., a host plant) by any known and applicable means. It is noted that the entirety of a removed symbiont, for use as a symbiont forming inoculum may not be necessary to achieve desired results. For example, only some stable material (e.g., one or more cells) from a symbiont may be removed and used for affixing to/transplanting to the host plant. In such a method, cells of a single symbiont removed and placed into culture may be propagated to provide material to transplant onto multiple host plants (e.g., as a symbiont forming inoculum). Techniques are well-known in the art for transplanting one plant or plant part such as a symbiont or symbiont forming inoculum onto another plant (e.g., a host plant) and can be used in the present invention. Preferentially, a symbiont forming inoculum may be affixed to a host plant such that the symbiont that is formed is in functional communication with the vascular system of the host plant, or such that functional communication with the vascular system of the host plant is achievable. Transplanting methods may allow for the symbiont tissue to develop the necessary vascular connections following transplantation, even if those connections are not established concurrently with transplantation. In this way, the desired trait/compound produced by the symbiont may travel through the vascular system of the second plant. In some embodiments, a desired trait/compound produced by the symbiont may be transported to the host plant via the apoplast and/or the symplast. In some embodiments, a desired trait/compound produced by a symbiont may be transported to a host plant via the apoplast, symplast or vascular system that develops between the host plant and symbiont, or via any combination thereof.

The first (original plant on which or from which the symbiont is grown or developed) and second plant (e.g., host plant onto which a symbiont forming inoculum may be transplanted) may be of the same species or a different species, depending on the specific interoperability (i.e. transplant compatibility) of plant material between the different species.

In some embodiments, activated cells/tissue may be formed by inoculation of a plant with at least one activated strain of Agrobacterium spp. as described above, the activated cells/tissue may be removed from a first plant and then cultured in a solution containing at least one trait-inducing Agrobacterium spp. strain. After sufficient uptake of the nucleic acid (e.g., T-DNA) from the trait-inducing strain, plant cells containing both polynucleotide(s) encoding a phytohormone biosynthetic enzyme and a trait-inducing nucleic acid (POI) (e.g., a symbiont forming inoculum) may exist in the culture. These cells (e.g., symbiont forming cells or inoculum) may be selected for by known methods, and then used as desired. For example, the cells may be selected for, removed and cultured to create more symbiont forming inoculum (e.g., bacterial cell population, callus tissue and/or a suspension culture comprising two or more cells) having the trait of interest. Alternatively, or additionally, the selected cells of a symbiont forming inoculum may be then used as described above (i.e. sterilized, transplanted onto a second plant (e.g., host plant, etc.).

In some embodiments, bacteria comprising at least one pSYM (e.g., a polynucleotide encoding at least one phytohormone biosynthetic enzyme and at least one POI) (e.g., one or more Agrobacterium spp. cells comprising at least one pSYM) may be directly delivered to (inoculated onto) a host plant. In some embodiments, the symbiont forming inoculum may include one Agrobacterium spp. strain or more than one Agrobacterium spp. strain as described above (e.g., one strain comprising one or more polynucleotides encoding at least one phytohormone biosynthetic enzyme and one strain comprising a POI or a single strain comprising both one or more polynucleotides encoding at least one phytohormone biosynthetic enzyme and a POI). In this way, the resulting symbiont tissue formed on the host plant would act as a beneficial biofactory for or on the host plant with regard to the desired molecule(s) without the need to transform the host plant. According to some embodiments, some or all of the inoculated strains may be engineered to be of low vitality such that once a useful symbiont (i.e. a symbiont that has hypervascularity and produces the desired molecule for the desired trait) forms on the plant, the bacteria die off and are no longer present in the symbiont.

In some embodiments, culturing a symbiont forming inoculum on a host plant can further comprise culturing under conditions that increase humidity. For example, a site on a host plant that is contacted with symbiont forming inoculum or onto which a symbiont is transplanted may be covered to increase humidity in the immediate area of the symbiont or symbiont forming inoculum. Any type of covering that retains humidity in the area surrounding the symbiont or symbiont forming inoculum that is transplanted onto the host plant may be used. As an example, symbiont or symbiont forming inoculum located on a site one a host plant may be encased or covered with a film to retain humidity. In some embodiments, the film can include, but is not limited to, a plastic film, silicon tape, and/or a parafilm. In some embodiments, a symbiont or symbiont forming inoculum on a host plant may be covered to increase humidity in the area of the symbiont or symbiont forming inoculum after (e.g., immediately after or within about 15 minutes to 5 hours after) the transplanting for about one hour to about 72 hours or more, about one hour to about 48 hours or more or about 1, 2, 3, 4, 5, 6, 7, 8, 9, 10 hours to about 11, 12, 13, 14, 15, 16, 17, 18, 19, 20, 21, 22, 23, 24, 25, 26, 27, 28, 29, 30, 31, 32, 33, 34, 35, 36 hours or more or any range or value therein. In some embodiments, a symbiont or symbiont forming inoculum on a host plant may be covered to increase humidity in the area of the symbiont or symbiont forming inoculum for about 10 hours to about 30 hours (e.g., about 10, 11, 12, 13, 14, 15, 16, 17, 18, 19, 20, 21, 22, 23, 24, 25, 26, 27, 28, 29, or 30 hours), optionally for about 24 hours after transplanting (host plant) or inoculating (inoculum formation).

In some embodiments, prior to or concurrently with transplanting at least one site a host plant, the host plant may be wounded at the least one site. Wounding can be carried out in any manner using any tools that are useful for breaking the outer surface (epidermis, cuticle, bark) of the plant or plant part at the site onto which the symbiont or symbiont forming inoculum is transplanted. Such tools can include, but are not limited to, a tweezer or forceps, a knife, a needle e.g., (e.g., hypodermic, dissecting, tattoo, sewing, and the like), a toothpick, and/or a syringe. In addition, any standard grafting tools may be utilized for introducing or transplanting as described herein.

In some embodiments, the at least one site on a host plant can be on an above ground part of the host plant and/or on a below ground part of the host plant.

In some embodiments, a symbiont is transplanted onto a host plant at least two times. In some embodiments, a symbiont forming inoculum is transplanted onto a host plant at least two times. In some embodiments, a symbiont and/or a symbiont forming inoculum is transplanted onto at least two sites on a host plant.

In some embodiments, an expression product of a polynucleotide of interest may be a transcription product or a translation product, or a modification thereof. As an example, the expression product of a polynucleotide of interest may be a methylation of a transcription product. In some embodiments, the expression product of a polynucleotide of interest may be, for example, a glycosylation of a translation product. A translation product may be a protein (polypeptide) or a peptide. A transcription product is a ribonucleic acid (RNA). In some embodiments, the RNA is a coding RNA (e.g., mRNA). In some embodiments, the RNA is a non-coding RNA including, but not limited to, transfer RNA (tRNA), ribosomal RNA (rRNA), small nuclear RNA (snRNA), small nucleolar RNA (snoRNA), piwi-interacting RNA (piRNA), microRNA (miRNA), long non-coding RNA (lncRNA), and/or small interfering RNA (siRNA).

In some embodiments, the expression product of the polynucleotide of interest may be a biosynthetic enzyme that may be used to make another product that can include, but is not limited to, a chemical, a protein (polypeptide/peptide) or a polynucleotide.

A modified host plant characteristic may include a modification of any plant characteristic including, but not limited to, a change in the metabolism of the host plant, a change in the structure of the host plant (e.g., morphology), and/or a change in the host plant's metabolism, biochemistry and/or physiology. In some embodiments, a modified host plant characteristic can be a change in the plant's response to, for example, a disease causing organisms such as, for example, a fungus, a bacteria, a virus and/or a protozoan. Thus, in some embodiments, a modified host plant characteristic can result in increased tolerance/resistance to a disease causing organism as compared to a plant not comprising the symbiont. A disease causing organism can include, but is not limited to, a fungus, a bacteria, a virus and/or a protozoan. In some embodiments, a modified host plant characteristic is increased (induced) expression of plant defense genes, thereby resulting in a host plant having increase disease resistance. In some embodiments, a plant defense gene that can be increased includes a “W-box” defense gene. A W-box defense gene can include, but not is limited to CAD1, NPR1, and/or PR2. In some embodiments, a plant defense gene can be increased in a host plant via a symbiont through the production of a chemical in the symbiont such as a chemical, which is transported to the host plant and which stimulates a systemic acquired resistance response in the host plant. In some embodiments, a plant defense gene can be increased in a host plant through the production in the host plant of a chemical that stimulates a systemic acquired resistance response in the host plant, wherein a biochemical pathway that produces the chemical in the host plant is modified by the product of the polynucleotide of interest in the symbiont that is transported to the host plant.

In some embodiments, a modified host plant characteristic can be a change in the plant's response to, for example, an insect or a nematode. Thus, in some embodiments, a modified host plant characteristic is increased insect tolerance/resistance as compared to a plant not comprising the symbiont. Insects for which tolerance or resistance can be increased include, but are not limited to, insects in the orders Lepidopteran, Coleopteran, Hemiptera, Thysanoptera, and Diptera. In some embodiments, a modified host plant characteristic is increased nematode tolerance/resistance as compared to a plant not comprising the symbiont. Nematodes for which tolerance or resistance can be increased include, but are not limited to, root knot nematode (Meloidogyne spp), cyst nematodes (Heterodera spp. and Globodera spp), root lesion nematodes (Pratylenchus spp.), burrowing nematode (Radopholus similis), reniform nematode (Rotylenchulus reniformis), grape nematode (Xiphinema index), and citrus nematode (Tylenchulus semipenetrans).

In some embodiments, a modified host plant characteristic can be a change in the plant's response to a bacterial pest. Thus, in some embodiments, a modified host plant characteristic is increased tolerance/resistance to a bacterial pest as compared to a plant not comprising the symbiont. The present invention provides methods and compositions that can increase resistance or tolerance to many bacterial pests including, but not limited to, Xanthomonas axonopodis, Xanthomonas campestris, Erwinia amylovora, Erwinia carotovora, Candidatus Liberibacter asiaticus, Candidatus Liberibacter solanacearum, Pseudomonas syringae, Xylella fastidiosa, Dickeya solani, Dickeya dadantii, Pectobacterium carotovorum and/or Raistonia solanacearum.

In some embodiments, a modified host plant characteristic can be a change in the plant's response to an herbicide. Thus, in some embodiments, a modified host plant characteristic is increased herbicide tolerance/resistance as compared to a plant not comprising the symbiont. Example herbicides for which a host plant characteristic may be modified to have resistance or tolerance includes but is not limited to glyphosate, triazine, dicamba, 2,4-D, clopyralid, flumioxazin, carfentrozone-ethyl, sulfentrozon, lactofen, fomesafen, acifluorfen, mesotrione, sulcotrione, tembotrione, topramezone, picolinafen, clomazone, isoxaflutole, mefenacet, flufenacet, imazamox, imazapyr, imazethapyr, rimsulfuron, tribenuron-methyl, triasulfuron, nicosulfuron, sulfosulfuron, sulfometuron-methyl, mesosulfuron-methyl, azimsulfuron, amidosulfuron, cyclosulfamuron, flumetsulam, metosulam, florasulam, diclosulam, and/or thiencarbazone-methyl. Thus, in some embodiments, a modified host plant characteristic is increased herbicide tolerance/resistance as compared to a plant not comprising the symbiont. Increased herbicide tolerance/resistance in a host plant may be to one herbicide or may be to two or more different herbicides.

In some embodiments, a modified host plant characteristic can be a change in the plant's response to abiotic stress. In some embodiments, a modified host plant characteristic is increased tolerance to an abiotic stress as compared to a plant that does not comprise a symbiont of this invention. In some embodiments, the host plant may show increased tolerance to more than one abiotic stress tolerance (e.g., 1, 2, 3, 4, 5, or more abiotic stresses). The term “abiotic stress” as used herein refers to outside, nonliving, factors that can cause harmful effects to plants. Thus, as used herein, abiotic stress includes, but is not limited to, cold temperature that results in freezing, chilling, heat or high temperatures, drought, excessive water, high light intensity, low light intensity, high ultra violet light, salinity, ozone, and/or combinations thereof. Parameters for the abiotic stress factors are species specific and even variety specific and therefore vary widely according to the species/variety exposed to the abiotic stress. Thus, while one species may be severely impacted by a high temperature of 23° C., another species may not be impacted until at least 30° C., and the like. Temperatures above 30° C. result in dramatic reductions in the yields of most important crops. This is due to reductions in photosynthesis that begin at approximately 20-25° C., and the increased carbohydrate demands of crops growing at higher temperatures. The critical temperatures are not absolute, but vary depending upon such factors as the acclimatization of the crop to prevailing environmental conditions. In addition, because most crops are exposed to multiple abiotic stresses at one time, the interaction between the stresses affects the response of the plant. For example, damage from excess light occurs at lower light intensities as temperatures increase beyond the photosynthetic optimum. Water stressed plants are less able to cool overheated tissues due to reduced transpiration, further exacerbating the impact of excess (high) heat and/or excess (high) light intensity. Thus, the particular parameters for high/low temperature, light intensity, drought and the like, which impact crop productivity will vary with species, variety, degree of acclimatization and the exposure to a combination of environmental conditions.

An “increased tolerance to abiotic stress” as used herein refers to the ability of a plant or part thereof comprising a symbiont of the present invention that is exposed to abiotic stress to withstand a given abiotic stress better than a control plant or part thereof (i.e., a plant or part thereof that has been exposed to the same abiotic stress and does not comprise the symbiont). Increased tolerance to abiotic stress can be measure using a variety of parameters including, but not limited to, the size and number of plants or parts thereof, and the like (e.g., number and size of fruits), the level or amount of cell division, the amount of floral abortion, the amount of sunburn damage, crop yield, and the like. Thus, in some embodiments of this invention, a plant or part thereof comprising a symbiont of the present invention, and having increased tolerance to the abiotic stress, for example, would have reduced flower abortion as compared to a plant or part thereof exposed to the same stress but which does not comprise the symbiont. Accordingly, in some embodiments, expression of a polynucleotide of interest in a symbiont can confer increased abiotic stress tolerance on a host plant. In some embodiments, the presence of a biomolecule and/or bioactive molecule produce by the symbiont and transported to the host plant can confer increase abiotic stress tolerance on the host plant, thereby modifying a host plant characteristic.

In some embodiments a modified host plant characteristic is a modification of host plant morphology. A symbiont as described herein comprising and expressing a polynucleotide of interest may be utilized to alter any plant structure including but not limited to leaves, stems, flowers, roots, buds, seeds, meristems, fruit, tubers, and the like. In some embodiments, a modified morphology comprises but is not limited to shortened internodes, increased lateral branching and/or increased flowering, as compared to a plant not comprising the symbiont.

In some embodiments, a modified host plant characteristic is the presence of a biomolecule, a bioactive molecule and/or a polypeptide involved in the biosynthesis of a biomolecule and/or bioactive molecule, wherein the biomolecule, the bioactive molecule and/or the polypeptide involved in the biosynthesis of a biomolecule and/or bioactive molecule is encoded by the polynucleotide of interest or results from the expression of the polynucleotide of interest (e.g., the polynucleotide of interest encodes a polypeptide or a regulatory nucleic acid that influences the production of the bioactive molecule in the plant) that can then be transported into the host plant, thereby modifying a host plant characteristic, wherein the modified host characteristic can include the presence of the biomolecule and/or bioactive molecule and/or can be a result of the presence of the biomolecule and/or bioactive molecule. As described herein, a symbiont formed on a plant develops a vascular system that connects with that of the host plant. In some embodiments, a biomolecule and/or bioactive molecule produced in the symbiont (e.g., expressed by the polynucleotide of interest) may be transported to the host plant via the connected vascular system or tissue. In some embodiments, transport of the biomolecule and/or bioactive molecule from the symbiont to the host plant may be systemic transport. In some embodiments, a biomolecule and/or bioactive molecule produced in a symbiont may be transported to the host plant via the apoplast and/or the symplast of the connected tissue between the symbiont and the host plant. In some embodiments, transport of a biomolecule and/or bioactive molecule from a symbiont to a host plant may be via any combination of the connected vascular system, the apoplastic pathway and/or the symplastic pathway of the symbiont and the host plant.

Thus, in some embodiments, a polynucleotide of interest that encodes a biomolecule and/or bioactive molecule is comprised in a symbiont of this invention that is transplanted onto a host plant, wherein the polynucleotide of interest is expressed in the symbiont and the biomolecule and/or bioactive molecule is transported to the host plant.

In some embodiments, a biomolecule and/or bioactive molecule can include, but is not limited to, a pharmaceutical, a biostimulant, a biofungicide, a bioherbicide, an insecticidal protein/peptide, a trypsin modulating oostatic factor (TMOF); a Bacillus thuringiensis toxin, a vegetative insecticidal protein (Vip), a nutrient, a plant growth regulator, an RNAi, a plantibody, a stylet sheath inhibitory protein, a ribozyme, a bacteriocin, a plant lipid, a plant fatty acid, a plant oil, an antimicrobial peptide, an aptamer, a CRISPR-Cas system polypeptide and a corresponding CRISPR guide nucleic acid, a zinc finger nuclease (ZFN), a Transcription Activator-Like Effector Nuclease (TALEN) and/or an engineered meganuclease. A “biomolecule” is any molecule produced by a living organism and/or part thereof (e.g., a cell or cell free system)). Thus, a biomolecule includes any molecule produced by a symbiont resulting, directly or indirectly, from a polynucleotide of interest comprised in and expressed in the symbiont, and optionally transported to a host plant to which the symbiont is affixed or attached. A biomolecule may also refer to a biomolecule (e.g., a second biomolecule) produced in the host plant as a result of transport into the host plant of a different biomolecule (e.g., a first biomolecule) that is expressed from the POI in the symbiont (e.g., the POI may encode a enzyme involved in the biosynthesis of a biomolecule that is then produced in the host plant utilizing that biosynthetic enzyme). A “biomolecule” includes, but is not limited to, a “bioactive molecule”. Bioactive molecules include any biomolecule that comprises biological activity of which many non-limiting examples are described herein.

A “pharmaceutical” as used herein, includes, but is not limited to, a therapeutic protein, a therapeutic polynucleotide, and/or a therapeutic chemical. In some embodiments, a pharmaceutical can include, but is not limited to, a vaccine, an antibody, a recombinant antibody, an antibody fragment, a fusion protein, an antibody fusion protein, human serum albumin, gastric lipase, insulin, glucocerebrosidase, growth factor, a cytokine, hepatitis B surface antigen (HBsAg)), Apo-A1, alpha-galactosidase (PRX-102), alpha-galactosidase (PRX-102), acetylcholinesterase (PRX-105), antitumor necrosis factor (Pr-anti-TNF), IgG, interferon-alpha, plasmin, lactoferrin, lysozyme, and/or collagen.

Example bacteriocins that may be encoded in a polynucleotide of interest can include but is not limited to, acidocin, actagardine, agrocin, alveicin, aureocin, aureocin A53, aureocin A70, bisin, carnocin, carnocyclin, caseicin, cerein, circularin A, colicin, curvaticin, divercin, duramycin, enterocin, enterolysin, epidermin/gallidermin, erwiniocin, gardimycin, gassericin A, glycinecin, halocin, haloduracin, klebicin, lactocin S, lactococcin, lacticin, leucoccin, lysostaphin, macedocin, mersacidin, mesentericin, microbisporicin, microcin S, mutacin, nisin, paenibacillin, planosporicin, pediocin, pentocin, plantaricin, pneumocyclicin, pyocin, reutericin 6, sakacin, salivaricin, sublancin, subtilin, sulfolobicin, tasmancin. thuricin 17, trifolitoxin, variacin, vibriocin, warnericin, and/or warnerin.

Additional antimicrobial peptides useful with this invention that may be encoded by a polynucleotide of interest include Gramicidin (AVGALAVVVWLWLWLW SEQ ID NO:35), Magainin 2 (GIGKFLHSAKKFGKAFVGEIMNS SEQ ID NO:36), LL-37 (cathelicidin) (LGDFFRKSKEKIGKEFKRIVQRIKFLRNLVPRTES SEQ ID NO:37), Pyrrhocoricin (PrAMP) (VDKGSYLPRPTPPRPIYNRN SEQ ID NO:38), Nisin A (lantibiotic) (ITSISLCTPGCKTGALMGCNMKTATCHCSIHVSK SEQ ID NO:39), HNP1 (α-defensin) (ACYCRIPACIAGERRYGTCIYQGRLWAFCC SEQ ID NO:40), TAP (β-defensin) NPVSCVRNK (GICVPIRCPGSMKQIGTCVGRAVKCCRKK SEQ ID NO:41), Plectasin (GFGCNGPWDEDDMQCHNHCKSIKGYKGGYCAKGGFVCKCY SEQ ID NO:42), Colistin (XTXXKLLXXT SEQ ID NO:43)(X=2,4-diaminobutanoic acid), Daptomycin (WNDTGKDADGSEY SEQ ID NO:44), Microcin J25 (VGIGTPIFSYGGGAGHVPEYF SEQ ID NO:45), Alamethicin (peptaibol) (PBABAQBVBGLBPVBBEQ SEQ ID NO:46)(B=α-aminoisobutyric acid), Gramicidin (SVKLFPVKLFP SEQ ID NO:47), Subtilosin A (NKGCATCSIGAACLVDGPIPDFEIAGATGLFGLWG SEQ ID NO:48), Kalata 1 (cyclotide)(GLPVCGETCVGGTCNTPGCTCSWPVCTRN SEQ ID NO:49), Rhesus θ-defensin 1 (RTD-1) ( ) (GFCRCLCRRGVCRCICTR SEQ ID NO:50)

Example bioinsecticides that may be encoded by the polynucleotide of interest include jaburetox (e.g., SEQ ID NO:24, polypeptide SEQ ID NO:25), trypsin modulating oostatic factor (TMOF) (e.g., SEQ ID NO:26; polypeptide SEQ ID NO:27, 28), a Bacillus thuringiensis toxin (e.g., δ endotoxins, e.g., Cry (crystal) toxin, Cyt (cytotoxic) toxin) (e.g., SEQ ID NO:33; polypeptide SEQ ID NO:34); a stylet sheath inhibitory protein (e.g., ficin (e.g., SEQ ID NO:51), bromelain), and/or a vegetative insecticidal protein (Vip). These are well known polypeptides. Bacillus thuringiensis toxins include, for example, the Cry (crystal) toxins (e.g., Cry I, Cry II, Cry III, Cry IV), the Cyt (cytotoxic) toxins, vegetative insecticidal proteins (Vip), which are classified into four families Vip1, Vip2, Vip3 and Vip4 according to their degree of amino acid similarity, and secreted insecticidal protein (Sip) toxins. These proteins include toxins having varying ranges of toxicity that can be broad or narrow (e.g., toxic only to a particular group of insects).

In some embodiments, a bioactive molecule encoded by the polynucleotide of interest is jaburetox (peptide JBTX), trypsin modulating oostatic factor (TMOF), a B. thuringiensis 5 endotoxin, a Cry toxin, a Cyt toxin, a leghemoglobin, a nitrogenase, ficin, bromelain, a bacteriocin, nisin, oncocin and/or oncocin analogs (e.g., SEQ ID NO:29, SEQ ID NO:30, SEQ ID NO: 31, SEQ ID NO: 32).

In some embodiments, a modified host plant characteristic is the presence a bioactive molecule (e.g., a biocidal molecule) and increased resistance/tolerance to a plant pathogen as compared to a plant not comprising the symbiont and the presence of a bioactive molecule transported into the plant from the symbiont. In some embodiments, the biocide is a bacteriocin or an antimicrobial peptide and the plant pathogen is a bacterium. In some embodiments, the bacteriocin or antimicrobial peptide is oncocin and/or nisin.

In some embodiments, a modified host plant characteristic is the presence of an insecticidal protein (e.g., a bioinsecticide) and increased insect tolerance or resistance as compared to a plant not comprising the symbiont and the presence of the insecticidal protein transported into the plant from the symbiont. In some embodiments, the insecticidal protein is jaburetox, trypsin modulating oostatic factor (TMOF), a Bacillus thuringiensis toxin (e.g. an 5 endotoxins), optionally a Cry (crystal) toxin, Cyt (cytotoxic) toxin, a vegetative insecticidal protein (Vip) or a secreted insecticidal protein (Sip) toxin and/or a stylet sheath inhibitory protein, optionally a ficin and/or bromelain.

In some embodiments, a stylet sheath inhibitory protein that may be expressed by a polynucleotide of interest in a symbiont of the invention. Such inhibitory peptides are known as exemplified in U.S. Patent Application No. 2018/0199577. Example stylet sheath inhibitory peptides useful for expression in symbionts include, but are not limited to, those listed in Table 3.

Table 4 provides an exemplary list of plants and example diseases or pests (e.g., insect and/or nematode pests) to which the plants are vulnerable. In some embodiments, the present invention may be used to provide increased tolerance/resistance in plants to these diseases and pests.

In some embodiments, a modified host plant characteristic is the presence of or increased or decreased production of a plant lipid, a plant fatty acid, and/or a plant oil.

In some embodiments, a modified host plant characteristic is the presence of or increased or decreased production of a plant growth regulator (e.g., auxin, cytokinin, gibberellin, ethylene; a growth inhibitor/retardant) and modified growth. In some embodiments, the modified growth may be increased growth or decreased growth of the host plant and/or increased or decreased growth of a part of a host plant as a result of transport of the growth regulator into the host plant from the symbiont, or as a result of the transport of a bioactive molecule into the host plant from the symbiont that results in increased or decreased production of the growth regulator in the host plant (e.g., a phytohormone biosynthetic enzyme). The increased or decreased production of a plant growth regulator and modified growth is as compared to a control plant (e.g., a plant not comprising the symbiont and the presence of the plant growth regulator and/or a plant not comprising the symbiont and the increased or decreased production of the plant growth regulator) In some embodiments, a modified host plant characteristic is the presence of or increased production of an RNA and increased/decreased production of a polynucleotide, a peptide or a polypeptide. An RNA useful with this invention may be any RNA that may be used to modify a plant characteristic, such as any RNA used for RNA interference (RNAi). In some embodiments, the RNA can include but is not limited to a siRNA, a dsRNA, a miRNA, and/or a shRNA. Exemplary RNAs include dvsnf7, ccomt, dCS, asn1, phL, RI, PGAS, and/or ppo5.

The present invention further provides a host plant having a modified characteristic produced by the methods of the invention.

Also provided herein is a method of producing a biomolecule or a bioactive molecule, the method comprising providing a symbiont of this invention, wherein the polynucleotide of interest encodes a biomolecule and/or bioactive molecule and collecting the biomolecule and/or bioactive molecule produced in the symbiont or symbiont forming inoculum; and/or providing a host plant of this invention, wherein the polynucleotide of interest encodes a biomolecule and/or a bioactive molecule and collecting the biomolecule and/or bioactive molecule produced in the symbiont forming inoculum and/or the symbiont and/or host plant.

Additionally provided is a method of delivering a compound of interest to a host plant, comprising transplanting a symbiont forming inoculum of this invention or a symbiont of this invention onto at least one site (e.g., 1, 2, 3, 4, 5, 6, 7, 8, 9, 10 or more sites) on a host plant; and culturing the symbiont forming inoculum or symbiont at the at least one site on the host plant to form a symbiont on the host plant at the at least one site, wherein the polynucleotide of interest is expressed in the symbiont and an expression product of the polynucleotide of interest and/or a product made using the expression product of a polynucleotide of interest is transported into the host plant, thereby delivering the compound of interest to a plant.

A method of producing a plant comprising a modified characteristic without modifying the plant's genotype is also provided, the method comprising: transplanting a symbiont forming inoculum of the present invention or a symbiont of the present invention onto at least one site (e.g., 1, 2, 3, 4, 5, 6, 7, 8, 9, 10 or more sites) on a host plant; and culturing the symbiont forming inoculum or symbiont at the at least one site on the host plant to form a symbiont on the host plant at the at least one site, wherein the polynucleotide of interest is expressed in the symbiont and an expression product of the polynucleotide of interest and/or a product made using the expression product of the polynucleotide of interest is transported into the host plant, thereby producing the plant comprising a modified phenotype without a modified genotype. A plant produced by the methods of the invention is also provided.

As described herein, a polypeptide encoded by a polynucleotide of this invention (e.g., a polypeptide encoded by a polynucleotide of interest, a phytohormone biosynthetic enzyme) may be operably linked to a targeting sequence. In some embodiments, a polypeptide may be linked to a targeting sequence at its N-terminus or its C-terminus or both. A targeting sequence useful with this invention may be any targeting sequence that can direct/locate a polypeptide or peptide to a specific organelle or plant part. A targeting sequence may be operably linked at the N- or C-terminus of a polynucleotide or nucleic acid molecule, optionally wherein the polynucleotide or nucleic acid molecule is heterologous to the targeting sequence. Targeting (or signal) sequences or targeting peptides (and the nucleotide sequences encoding them) are well known in the art and can be found in public databases such as the “Signal Peptide Website: An Information Platform for Signal Sequences and Signal Peptides.” (www.signalpeptide.de); the “Signal Peptide Database” (proline.bic.nus.edu.sg/spdb/index) (Choo et al., BMC Bioinformatics 6:249 (2005)(available on biomedcentral.com/1471-2105/6/249/abstract); ChloroP (cbs.dtu.dk/services/ChloroP/; predicts the presence of chloroplast transit peptides (cTP) in protein sequences and the location of potential cTP cleavage sites); LipoP (cbs.dtu.dk/services/LipoP/; predicts lipoproteins and signal peptides in Gram negative bacteria); MITOPROT (ihg2.helmholtz-muenchen.de/ihg/mitoprot; predicts mitochondrial targeting sequences); PlasMit (gecco.org.chemie.uni-frankfurt.de/plasmit/index; predicts mitochondrial transit peptides in Plasmodium falciparum); Predotar (urgi.versailles.inra.fr/predotar/predotar.html; predicts mitochondrial and plastid targeting sequences); PTS1 (mendel.imp.ac.at/mendeljsp/sat/pts1/PTS1predictor.jsp; predicts peroxisomal targeting signal 1 containing proteins); SignalP (cbs.dtu.dk/services/SignalP/; predicts the presence and location of signal peptide cleavage sites in amino acid sequences from different organisms: Gram-positive prokaryotes, Gram-negative prokaryotes, and eukaryotes). The SignalP method incorporates a prediction of cleavage sites and a signal peptide/non-signal peptide prediction based on a combination of several artificial neural networks and hidden Markov models; and TargetP (cbs.dtu.dk/services/TargetP/); predicts the subcellular location of eukaryotic proteins—the location assignment is based on the predicted presence of any of the N-terminal presequences: chloroplast transit peptide (cTP), mitochondrial targeting peptide (mTP) or secretory pathway signal peptide (SP)). (See also, von Heijne, G., Eur J Biochem 133 (1) 17-21 (1983); Martoglio et al. Trends Cell Biol 8 (10):410-5 (1998); Hegde et al. Trends Biochem Sci 31(10):563-71 (2006); Dultz et al. J Biol Chem 283(15):9966-76 (2008); Emanuelsson et al. Nature Protocols 2(4) 953-971(2007); Zuegge et al. 280(1-2):19-26 (2001); Neuberger et al. J Mol Biol. 328(3):567-79 (2003); and Neuberger et al. J Mol Biol. 328(3):581-92 (2003)). Example targeting sequences useful for targeting polypeptides as described herein include, but are not limited to, those provided in Table 5. In some embodiments, a polypeptide encoded by a POI may be operably linked to a sequence that targets the secretory system (e.g., the endoplasmic reticulum (ER), e.g., an ER targeting sequence).

As described herein, a plant, plant part or plant cell useful with embodiments of this invention may be any plant or from any plant including but not limited to, an angiosperm (e.g., a dicot plant or a monocot plant), gymnosperm, an algae (e.g., a macroalgae, e.g., Rhodophyta (red algae), Phaeophyta (brown algae) and Chlorophyta (green algae), Chrysophyceae (gold algae)), a bryophyte, fern and/or fern ally (i.e., pteridophyte).

A plant useful with this invention (e.g., for symbiont forming inoculum, a symbiont, a plant, or a host plant as described herein) can include, but is not limited to, any plant from the genera of Abelia spp. (Abelia), Abelmoschus spp. (Okra), Abies spp. (Fir), Acacia spp. (Acacia), Acalypha spp. (Chenille), Acca spp. (Feijoa, pineapple guava, guavasteen), Acer spp. (Maple), Achillea spp. (Yarrow), Achlys spp. (Barberry), Acmella spp. (Paracress), Acoelorrhaphe spp. (Palm), Acorus spp. (Calamus), Acronychia spp. (Aspen), Acrostichum spp. (Fern), Acrotriche spp. (Currant), Actinidia spp. (Kiwifruit), Adansonia spp. (Baobab), Adiantum spp. (Maidenhair Fern), Adonidia spp. (Palm), Aechmea spp. (Bromeliad), Aegle spp. (Bael), Aesculus spp. (Buckeye, Horse-chestnut), Aframomum spp. (False Cardamon), Agapanthus spp. (Agapanthus), Agaricus spp. (Mushroom), Agastache spp. (Anise), Agathosma spp. (Buchu), Agave spp. (Agave), Ageratum spp. (Whiteweed), Aglaonema spp. (Chinese evergreens), Agrimonia spp. (Agrimony), Ailanthus spp. (Tree-of-Heaven), Ajuga spp. (Bugle), Albizia spp. (Silk trees), Alchemilla spp. (Lady's mantle), Aleurites spp. (Candlenut), Allamanda spp. (Allamanda), Allium spp. (Chive, Garlic, Leek, Onion, Shallot), Alnus spp. (Alder), Alocasia spp. (Elephant's Ear), Aloe spp. (Aloe), Aloysia spp. (Beebrushes), Alpinia spp. (Shell ginger), Alternanthera spp. (Joyweeds), Althaea spp. (Marshmallow), Amaranthus spp. (Amaranth), Amelanchierspp. (Juneberry, Serviceberry), Amomum spp. (Cardamom), Amphitecna spp. (Black Calabash), Anacardium spp. (Cashew), Ananas spp. (Pineapple), Anaphalis spp. (Pearly everlasting), Andrographis spp. (False waterwillows), Andromeda spp. (Bog rosemary), Anethum spp. (Dill), Angelica spp. (Angelica), Angelonia spp. (Angelonia), Angostura spp. (Angostura), Annona spp. (Cherimoya, Sweetsop, Sugar-apple, Soursop), Anogeissus spp. (Axlewood), Anthemis spp. (Chamomile), Anthoxanthum spp. (Grass), Anthriscus spp. (Chervil), Anthurium spp. (Tailflower), Antidesma spp. (Bignay), Antigonon spp. (Coralvine), Antirrhinum spp. (Snapdragon), Apium spp. (Celery), Aquilegia spp. (Columbine), Arabidopsis spp. (Thale Cress, Mouse-ear Cress), Aralia spp. (Walkingstick, Udo), Araucaria spp. (Pine), Arbutus spp. (Madrone, Strawberry tree), Arctium spp. (Burdock), Arctostaphylos spp. (Bearberry, Manzanita), Ardisia spp. (Marlberry), Armeria spp. (Thrift), Armoracia spp. (Horseradish), Aronia spp. (Aronia), Arracacia spp. (Arracacha), Artemisia spp. (Wormwood), Artocarpus spp. (Breadfruit, Jackfruit, Monkeyfruit), Aruncus spp. (Goat's Beard), Arundinaria spp. (Bamboo), Asarum spp. (Ginger), Asclepias spp. (Milkweed), Ascophyllum sp. (Feamainn Bhui, Rockweed, Norwegian Kelp, Knotted Kelp, Knotted Wrack or Egg Wrack), Asimina spp. (Pawpaw), Aspalathus spp. (Rooibos), Asparagus spp. (Asparagus, Asparagus Fern), Aspidistra spp. (Cast Iron Plant), Aspidosperma spp. (Quebracho), Asplenium spp. (Nest Fern), Aster spp. (Aster), Astragalus spp. (Milkvetch), Asystasia spp. (Asystasia), Athyrium spp. (Lady Fern), Atriplex spp. (Orache), Auriculari spp. (Edible Fungi), Avena spp. (Oats), Averrhoa spp. (Starfruit), Baccaurea spp. (Lotkon), Baccharis spp. (Saltbush), Backhousia spp. (Ironwood), Bactris spp. (Peach Palm), Balanites spp. (Torchwood), Baleria spp. (Violet), Bambusa spp. (Bamboo), Baptisia spp. (Indigo), Barbarea spp. (Cress), Basella spp. (Spinach), Bauhinia spp. (Orchid Tree), Beaucarnea spp. (Palm), Begonia spp. (Begonia), Belamcanda spp. (Lily), Benincasa spp. (Waxgourd), Berberis spp. (Barberry), Bertholletia spp. (Brazil Nut), Beta spp. (Beet, Swiss Chard), Betula spp. (Birch), Bidens spp. (Beggarticks), Billardiera spp. (Appleberry), Bischofia spp. (Bishopwood), Bismarckia spp. (Palm), Bixa spp. (Annatto), Blighia spp. (Ackee), Boesenbergia spp. (Fingerroot), Borago spp. (Borage), Borassus spp. (Palm), Borojoa spp. (Borojó), Borrichia spp. (Sea Oxeye), Boscia spp. (Hanza), Boswellia spp. (Frankincense), Bouea spp. (Plum mango), Brahea spp. (Palms), Brassica spp. (Broccoli, Swiss Chard, Cabbage, Cauliflower, Choy sum, Kale, Mustard, Mustard greens, Rapeseed, Rutabaga, Brussel Sprout), Breynia spp. (Snowbush), Brosimum spp. (Breadnut), Browallia spp. (Amethyst Flower), Brunfelsia spp. (Raintree), Buchanania spp. (Chirauli-nut), Bucida spp. (Bullet tree), Bumelia spp. (Chittamwood), Bunchosia spp. (Peanut butter fruit), Bursera spp. (Limbo), Butia spp. (Jelly palm), Buxus spp. (Boxwood), Byrsonima spp. (Locustberry), Caesalpinia spp. (Caesalpinia), Cajanus spp. (Pigeon pea), Caladium spp. (Caladium), Calamagrostis spp. (Reed grass, smallweed), Calathea spp. (Calatheas, prayer plants, Leren), Calendula spp. (Marigold), Calliandra spp. (Powder puff plant, fairy duster), Callicarpa spp. (Beautyberry), Callistemon spp. (Bottlebrushes), Calocedrus spp. (Incense cedar), Calophyllum spp. (Calophyllum), Calycanthus spp. (Sweetshrub), Calyptranthes spp. (Lidflowers, spicewoods, mountainbays), Camassia spp. (Camas, wild hyacinth), Camelina spp. (False flax, Camellia), Campanula spp. (Bellflower), Campomanesia spp. (Guabiroba), Campsis spp. (trumpet creeper, hummingbird vine), Canarium spp. (Pacific almond), Canavalia spp. (Jackbeans), Canella spp. (Cinnamon bark), Canna spp. (Canna Lily, Indian shot), Cannabis spp. (Cannabis), Capparis spp. (Caperbushes), Capsella spp. (Shepherd's purse), Capsicum spp. (Bell peppers, Cayenne pepper, Chile Jalapeno, Pepper), Carex spp. (True sedges), Carica spp. (Papaya), Carissa spp. (Natal Plum, num-num), Carnegiea spp. (Saguaro), Carpentaria spp. (Carpentaria palm), Carpinus spp. (Hornbeams), Carpobrotus spp. (Pigface, ice plant, sour fig, Hottentot fig), Carthamus spp. (Safflower), Carum spp. (Caraway), Carya spp. (Hickory nut, Pecan), Caryocarspp. (Pequi, Souari-nut), Caryota spp. (Fishtail palms), Casasia spp. (Casasia, Seven-year Apple), Casimiroa spp. (Sapote), Cassia spp. (Cassia), Castanea spp. (Chestnut, Chinquapin), Casuarina spp. (Australian pine), Casuarinaceae spp. (Sheoak), Catalpa spp. (Catalpa, catawba), Catharanthus spp. (Periwinkles), Ceanothus spp. (Ceanothus, buckbrush, soap bush), Cedrus spp. (Cedar), Ceiba spp. (Silk-Floss Tree), Celosia spp. (Cockscombs, woolflowers), Celtis spp. (Hackberries, nettle trees), Centaurium spp. (Centaury), Centella spp. (Pennywort), Centratherum spp. (Brazilian Button, Lark Daisy), Cephalanthus spp. (Buttonbush), Cerastium spp. (Mouse-ear chickweed), Ceratonia spp. (Carob), Cercidiphyllum spp. (Katsura), Cercis spp. (Redbuds), Chaenomeles spp. (Quince), Chaerophyllum spp. (Chervil), Chamaecyparis spp. (Falsecypress), Chamaedorea spp. (Bamboo Palm, parlor palm), Chamaemelum spp. (Chamomile), Chamaerops spp. (European Fan Palm), Chelidonium spp. (Celandine), Chenopodium spp. (Goosefoots), Chilopsis spp. (Desert willow), Chimaphila spp. (Prince's pine), Chimonobambusa spp. (Bamboo), Chiococca spp. (Milkberry, Snowberry), Chionanthus spp. (Fringetree), Chrysanthemum spp. (Chrysanthemums), Chrysobalanus spp. (Cocoplum), Chrysophyllum spp. (Star apple, Satinleaf), Cicer spp. (Chickpea), Cichorium spp. (Chicory, Endive, Escarole), Cinchona spp. (Quina), Cinnamomum spp. (Cinnamon, Camphor tree, Cassia), Cirsium spp. (Thistle), Citharexylum spp. (Fiddlewood, zitherwood), Citrillus spp. (Watermelon), Citrus spp. (Citrus, Grapefruit, Lemon, Lime, Orange, Pummelo, Tangerine), Cladrastis spp. (Yellowwood), Clarkia spp. (Godetia), Clausena spp. (Wampi), Claytonia spp. (Purslane), Cleome spp. (Spider plant, bee plant, Cat's Whiskers), Clerodendron spp. (Glorybower, bagflower, bleeding-heart), Clinopodium spp. (Calamint), Clusiarosea spp. (Clusia, Pitch Apple), Coccoloba spp. (Seagrape), Coccothrinax spp. (Silverpalm), Cocos spp. (Coconut), Coffea spp. (Coffee), Coleus spp. (Coleus), Colocasia spp. (Taro), Colubrina spp. (Nakedwood, snakewood, greenheart, hogplum), Combretum spp. (Bushwillows, Combretum), Commiphora spp. (Myrrh), Conocarpus spp. (Buttonwood), Conradina spp. (False rosemary), Conringia spp. (Hare's ear Mustard), Convallaria spp. (Lily-of-the-valley), Copaifera spp. (Copaiba), Coptis spp. (Goldthread), Corchorus spp. (Jute), Cordia spp. (Manjack, bocote), Cordyline spp. (Ti Plant, palm lily), Coreopsis spp. (Calliopsis, tickseed), Coriandrum spp. (Coriander, cilantro), Cornus spp. (Dogwood), Coronilla spp. (Crownvetch), Corydalis spp. (Corydalis), Corylus spp. (Filbert, Hazel, Hazelnut), Cosmos spp. (Cosmos, Mexican aster, Kenikir), Costus spp. (Spiral ginger), Cotinus spp. (Smoketree), Crambe spp. (Crambe), Crassocephalum spp. (Ragleaf, thickhead, bologi, Ebolo), Crassula spp. (Jade plant, pygmyweed), Crataegus spp. (Hawthorn, quickthorn, thornapple, May-tree, hawberry), Crescentia spp. (Calabash tree, huingo, krabasi, kalebas), Crinum spp. (Swamplily, Crinum), Crocus spp. (Saffron), Crotalaria spp. (Chipilin), Cryptotaenia spp. (Honewort, Japanese cedar), Cucumis spp. (Cantaloupe, Cucumber, Melon, Gherkin, Muskmelon, Honeydew), Cucurbita spp. (Pumpkin, Summer Squash, Winter Squash), Cuminum spp. (Cumin), Cunninghamia spp. (Cunninghamia, China-fir), Cupaniopsis spp. (Tuckeroo, soapberry), Cuphea spp. (Cuphea, cigar plant, Heather), Cupressocyparis spp. (Leylandii, Leyland cypress), Cupressus spp. (Cypress), Curcuma spp. (Turmeric), Cyamopsis spp. (Guar), Cycas spp. (Cycas), Cyclopia spp. (Honeybush), Cydonia spp. (Quince), Cymbopogon spp. (Lemongrass), Cynara spp. (Cardoon, artichoke, thistle), Cyperus spp. (Chufa, Papyrus sedges, flatsedges, nutsedges, umbrella-sedges, galingale), Dahlia spp. (Dahlia), Dalbergia spp. (Kingwood, Indian rosewood, African blackwood, tulipwood), Daucus spp. (Carrot), Davidsonia spp. (Ooray), Delonix spp. (Poinciana), Dendranthema spp. (Mums), Dendrocalamus spp. (Bamboo), Deparia spp. (Fern), Dermatophyllum spp. (Mescalbean), Deschampsia spp. (Hair grass, tussock grass), Dialium spp. (Tamarind), Dianthus spp. (Carnation, pink, sweet william, Dianthus), Dicentra spp. (Bleeding-hearts), Dictyophora spp. (Stinkhorn), Dietes spp. (Wood iris, fortnight lily, African iris, Japanese iris, butterfly iris), Dimocarpus spp. (Longan), Dioscorea spp. (Yam), Diospyros spp. (Persimmon, Black sapote), Diplazium spp. (Fern), Diplotaxis spp. (Wild rocket), Dizygotheca spp. (False aralia, Dizygotheca), Dodonaea spp. (Hop-bush), Doellingeria spp. (Cham-chwi), Dombeya spp. (Dombeya, dikbas, Pinkball), Dovyalis spp. (Gooseberry, Kei-apples), Dracaena spp. (Dragon tree, Dracaena), Dryopteris spp. (Fern), Durio spp. (Durian), Dypsis spp. (Butterfly palm), Dyschoriste spp. (Snakeherb), Dysphania spp. (Epazote), Echinacea spp. (Echinacea, coneflowers), Echium spp. (Paterson's curse), Ecklonia spp. (Ecklonia Cava), Elaeagnus spp. (Silverberry, oleaster), Elaeocarpus spp. (Ceylon olive), Elettaria spp. (Cardamom), Elwendia spp. (Black cumin), Elymus spp. (Couch grass, wildrye, wheatgrass), Epilobium spp. (Willowherbs), Epimedium spp. (Barrenwort), Epipremnum spp. (Centipede tongavine, pothos, devil's ivy), Eremocitrus spp. (Desert lime), Erigeron spp. (Fleabane, Erigeron), Eriobotrya spp. (Loquat), Eriodictyon spp. (Yerba santa), Ernodea spp. (Beech creeper, coughbush), Eruca spp. (Arugula), Eryngium spp. (Eryngo, sea holly, Culantro), Erythrina spp. (Coral tree, Flame tree, bucare, kafferboom), Eucalyptus spp. (Gums, eucalypts, Mallee), Eucharis spp. (Amazon lily), Eucommia spp. (Chinese rubber tree), Eugenia spp. (Dune myrtle, rainforest plum, mountain cherry, pitanga, Araza), Euodia spp. (Euodia), Eupatorium spp. (Boneset, thoroughworts, snakeroot), Euphorbia spp. (Spurge), Euryops spp. (Euryops), Eustoma spp. (Lisianthus, prairie gentian), Euterpe spp. (Agai palm), Exacum spp. (Persian violet), Fagopyrum spp. (Buckwheat), Fagus spp. (Beech), Fatshedera spp. (Tree ivy, aralia ivy), Ferula spp. (Fennel, Muskroot, Sumbul), Festuca spp. (Fescue), Ficaria spp. (Celandine), Ficus spp. (Fig), Filipendula spp. (Meadowsweet), Firmiana spp. (Parasol tree), Flacourtia spp. (Batoko plum), Flammulina spp. (Enokitake), Foeniculum spp. (Fennel), Forestiera spp. (Swampprivets), Fortunella spp. (Kumquat), Fothergilla spp. (Witch alder), Fragaria spp. (Strawberry), Frangula spp. (Cascara), Franklinia spp. (Franklin tree), Fraxinus spp. (Ash), Fritillaria spp. (Fritillaries), Fucus spp. (Rockweed), Fumaria spp. (Fumitory), Gaillardia spp. (Blanket flower), Galium spp. (Sweetscented bedstraw), Ganoderma spp. (Reishi Mushroom), Garberia spp. (Garberia, Garber's scrub starts), Garcinia spp. (Mangosteen, saptrees, garcinias), Gardenia spp. (Gardenia), Gaultheria spp. (Wintergreen, waxberry, snowberry, Shallon), Gaylussacia spp. (Huckleberry), Gazania spp. (Gazanlia, trailing gazania, clumping gazania), Geijera spp. (Geijera, wilga, oilbush, sheepbush), Genipa spp. (Genip), Gentiana spp. (Gentian), Geranium spp. (Geranium, cranesbill), Gigantochloa spp (Bamboo), Ginkgo spp. (Ginkgo, maidenhair tree), Glebionis spp. (Chrysanthemum, Corn Marigold, crown daisy), Gleditsia spp. (Honey locust), Glinus spp. (Sweetjuice), Glycine spp. (Soybean), Gomphrena spp. (Globe amaranth), Goodyera spp. (Rattlesnake plantain, jade orchids, ladies' tresses), Gordonia spp. (Gordonia, loblolly-bay), Gossypium spp. (Cottonseed), Grevillea spp. (Grevillea, spider flower, silky oak, toothbrush plant), Grewia spp. (Phalsa), Grifola spp. (Maitake), Grindelia spp. (Gumweed), Guaiacum spp. (Guaiac), Guizotia spp. (Niger seed), Gymnema spp. (Gymnema), Gymnocarpium spp. (Oak fern), Gymnocladus spp. (Coffeetree, soap tree), Hakonechloa spp. (Hakone grass, Japanese forest grass), Halesia spp. (Silverbell, snowdrop tree), Hamamelis spp. (Witch-hazel), Hamelia spp. (Firebush, hummingbird bush, scarlet bush, redhead), Hancornia spp. (Mangaba), Harpephyllum spp. (Kaffir-plum), Hedychium spp. (Garland flower, ginger lily, kahili ginger), Helianthus spp. (Sunflower, Jerusalem artichoke), Helichrysum spp. (Curry plant), Heliconia spp. (Lobster-claws, toucan beak, wild plantains, false bird-of-paradise), Helictotrichon spp. (Blue oat grass), Hemerocallis spp. (Daylily), Heracleum spp. (Hogweed, cow parsnip), Hericium spp. (Pom Pom, edible mushroom), Hesperis spp. (Dame's rocket), Heuchera spp. (coral bell, alumroot), Hibiscus spp. (Hibiscus, rose mallow, rose of sharon), Hierochloe spp. (Grass), Hippeastrum spp. (Amaryllis), Hippophae spp. (Sea buckthorn), Holodiscus spp. (Oceanspray, creambush), Hordeum spp. (Barley), Hosta spp. (Hosta, giboshi, plantain lilies), Houttuynia spp. (Dokudami), Hovenia spp. (Japanese Raisintree), Howea spp. (Kentia palm, thatch palm, curly palm), Hoya spp. (Waxplant, waxvine, waxflower, hoya), Hybrid spp. (Astilbe), Hydrangea spp. (Hydrangea, hortensia), Hydrophyllum spp. (Waterleaf), Hylocereus spp. (Dragonfruit, pitahaya), Hymenaea spp. (Courbaril), Hymenocallis spp. (Spider Lily), Hypericum spp. (St. John's wort, goatweed), Hyphaene spp. (Doum palm), Hypsizygus spp. (Beech Mushroom), Hyssopus spp. (Herb Hyssop), Ilex spp. (Holly, winterberry), Illicium spp. (Star anise, anisetree), Impatiens spp. (Impatiens, jewelweed, touch-me-not, snapweed, patience, balsam, busy lizzie), Imperata spp. (Satintails), Indigofera spp. (Indigo), Inga spp. (Inga), Ipomoea spp. (Sweet potato, morning glories, water convolvulus, kangkung, bindweed, moonflower, Jalap), Iris spp. (Iris), Irvingia spp. (Dika), Iva spp. (Marsh Elder), Ixora spp. (West Indian Jasmine, viruchi, rangan, kheme, ponna, chann tanea, techi, pan, siantan, jarum-jarum, jejarum, jungle flame, jungle geranium, cruz de Malta), Jacaranda spp. (Jacaranda), Jasminum spp. (Jasmine), Jatropha spp. (Physic nut, nettlespurge), Jubaea spp. (Palm), Juglans spp. (Walnut), Juncus spp. (Rush), Juniperus spp. (Juniper), Justicia spp. (Water-willow, shrimp plant, Malabar nut), Kalanchoe spp. (Kalanchoe, Panda plant, mother of thousands, felt plant), Kalimeris spp. (Indian aster, Kalimeris Aster), Kalmia spp. (Sheep-laurel, lamb-kill, calf-kill, kill-kid, sheep-poison, Spoonwood), Kalopanax spp. (Castor aralia, tree aralia, prickly castor oil tree), Kniphofia spp. (Tritoma, red hot poker, torch lily, knofflers, poker plant), Koelreuteria spp. (Goldenrain Tree, Flamegold, Chinese Flame-Tree), Kunzea spp. (Kunzea, kanuka, manuka, muntries), Lablab spp. (Hyacinth bean, lablab bean, bataw, Indian bean), Laburnum spp. (Golden chain, golden rain, Laburnum), Lactuca spp. (Lettuce, Celtuce), Lagenaria spp. (Calabash, Gourd), Lagerstroemia spp. (crepe myrtle, crape myrtle), Laminaria spp. (Kelp, Tangle), Lansium spp. (Lanzones, langsat), Latania spp. (Latan palm, latania palm), Launaea spp. (Launaea), Laurus spp. (Bay laurel, sweet bay), Lavandula spp. (Lavender), Lecythis spp. (Paradise nut, monkey pot, cream nut, sapucaia nut), Leea spp. (Leea, Talyantan), Lens spp. (Lentil), Lentinula spp. (Shiitake), Leonurus spp. (Motherwort), Lepidium spp. (Peppercress, peppergrass, pepperwort, tumbleweed), Lepista spp. (Blewitt, mushroom-forming fungi), Lespedeza spp. (Bush clover, Japanese clover), Lesquerella spp. (Gaslight bladderpod), Lessertia spp. (Balloon pea), Leucaena spp. (Leadtrees), Leucanthemum spp. (Max chrysanthemum, creeping daisy, oxeye daisy, Shasta daisy), Leucothoe spp. (Leucothoe, sweetbells, doghobble, black laurel), Leucothrinax spp. (Palm), Levisticum spp. (Lovage), Lewisia spp. (Lewisia), Liatris spp. (Blazing star), Licania spp. (Gopher apple, Sansapote, merecure, oiticica), Ligustrum spp. (Privet), Lilium spp. (True Lily, Lily), Limnanthes spp. (Meadowfoam), Limnophila spp. (Marshweeds), Limonia spp. (Wood-apple), Limonium spp. (Sea-lavender, statice, caspia, marsh-rosemary), Lindera spp. (Spicewood, spicebush, Benjamin bush), Linnaea spp. (Beauty bush, Twinflower), Linum spp. (Flax), Lippia spp. (Lippia, Mexican Oregano, Licorice verbena), Liquidambar spp. (American storax, satin-walnut, redgum, sweetgum, star gum), Liriodendron spp. (Tuliptree, tulip poplar, yellow poplar), Liriope spp. (Lilyturf, monkey grass, spider grass), Litchi spp. (Lychee), Livistona spp. (Fan palm), Lobelia spp. (Lobelia), Lobularia spp. (Sweet alyssum), Lonicera spp. (Honeysuckle), Loropetalum spp. (Loropetalum, Chinese fringe flower), Lotus spp. (Lotus, deervetch, bird's-foot trefoil, Trefoil), Luffa spp. (Gourd, loofah), Lunaria spp. (Honesty), Lupinus spp. (Lupin, lupine), Lychnis spp. (Campion, catchfly), Lycium spp. (Goji berry, box-thorn, desert-thorn), Lycopersicon spp. (Tomato, wild tomato), Lycopus spp. (Gypsywort, bugleweed, waterhorehound), Lyonia spp. (Staggerbush, Poor-grub, Maleberry, He-huckleberry, Hurrahbush), Lysichiton spp. (Skunk cabbage, swamp lantern), Lysiloma spp. (False tamarind, sabicu), Lysimachia spp. (Loosestrife), Maackia spp. (Maackia), Macadamia spp. (Macadamia), Maclura spp. (Cockspur thorn, Osage orange, Dyer's mulberry, mandarin melon berry. Che), Macrocystis spp. (Giant Kelp, Giant Bladder Kelp), Magnolia spp. (Magnolia), Mahonia spp. (Oregon grape, Frémont's mahonia, agarita, chaparral berry), Maianthemum spp. (False Solomon's seal), Malcolmia spp. (Virginia stock, African mustard), Mallotonia spp. (Sea Lavender), Malpighia spp. (Acerola, Barbados cherry, dwarf holly), Malus spp. (Apple, crabapples, crabtrees, wild apples), Mammea spp. (Mammee apple, tropical apricot, salapee), Mandevilla spp. (Rocktrumpet, Allamanda), Mangifera spp. (Mango, white mango, jack, pahutan, Paho), Manihot spp. (Cassava), Manilkara spp. (Sapodilla, massaranduba, chicle, sapota), Maranta spp. (Prayer plant, obedience plant, Maranta), Marlierea spp. (Beruquillo), Marrubium vulgare (Horehound), Matisia spp. (Molinillo, Chupa-chupa), Matricaria spp. (German chamomile, mayweed), Matteuccia spp. (Ostrich fern, fiddlehead fern, shuttlecock fern), Matthiola spp. (Stock, gilly-flower), Medicago spp. (Alfalfa, medick, burclover), Melaleuca spp. (Paperbark, honey-myrtle, tea-tree), Melampodium spp. (Blackfoot), Melia spp. (Chinaberry tree, Persian lilac), Melicoccus spp. (Mamoncillo, Motoyoé, Quenepa), Melilotus spp. (Melilot, sweet clover, kumoniga), Melissa spp. (Lemon balm), Mentha spp. (Mint), Merrillia spp. (Flowering merrillia, katinga, Malay lemon), Mesembryanthemum spp. (Ice plants), Mespilus spp. (Medlar), Metasequoia spp. (Dawn redwood), Michelia spp. (Michelia, White Champaca, Champak, Dandy, Banana Shrub), Microcitrus spp. (Lime), Micromeria spp. (Yerba buena, white micromeria, white-leaved savory, Micromeria), Milicia spp. (Iroko, African teak, odum), Millettia spp. (Pongamia), Mimulus spp. (Monkeyflower), Miscanthus spp. (Silvergrass, Maiden grass), Mitchella spp. (Partridge berry), Momordica spp. (Bitter melon, Gac, spine gourd, kantola, Balsam pear), Monarda spp. (Beebalm, horsemint, oswego tea, bergamot), Monstera spp. (Swiss Cheese plant, shingle plant, five holes plant, Monstera), Montia spp. (Miner's lettuce, water chickweed, Winter Purslane), Morchella spp. (Morel), Morinda spp. (Noni, Indian mulberry, sweet morinda, redgal, yawweed, cheese shrub), Morus spp. (Mulberry), Mucuna spp. (Deer-eye beans, donkey-eye beans, ox-eye beans, hamburger seed), Muhlenbergia spp. (Muhly), Muntingia spp. (Jamaica-cherry), Murraya spp. (Curry tree, orange jasmine, china box), Musa spp. (Banana, plantain), Myrcianthes spp. (Lucumillo, twinberry, Arrayen, Guabiyu), Myrciaria spp. (Jaboticaba, Guavaberry, hivapuru, sabare, ybapuru), Myrica spp. (Bayberry, bay-rum tree, candleberry, sweet gale, wax-myrtle), Myristica spp. (Nutmeg, Mace, Kumpang, Macassar nutmeg, silver nutmeg), Myroxylon spp. (Balsam), Myrrhis spp. (Cicely, myrrh, sweet chervil), Myrsine spp. (Colicwood, kolea, matipo), Myrtus spp. (Myrtle), Nandina spp. (Nandina, heavenly bamboo, sacred bamboo), Narcissus spp. (Daffodil, narcissus, jonquil), Nasturtium spp. (Watercress, yellowcress), Nastus spp. (Bamboo), Nelumbo spp. (Lotus), Neomarica spp. (Walking iris, apostle's iris, apostle plant), Nepeta spp. (Catnip, catmint, catswort), Nephelium spp. (Rambutan, Korlan, Pulasan), Nephrolepis spp. (Macho ferns, swordfern), Nerium spp. (Oleander, nerium), Nicotiana spp. (Tobacco plants), Nigella spp. (Black Caraway, nigella, devil-in-a-bush, love-in-a-mist), Noronhia spp. (Madagascar olive), Nymphaea spp. (Water Lily, waterlily), Nyssa spp. (Tupelo, Blackgum), Ochrosia spp. (Elliptic yellowwood, bloodhorn, kopsia, Kauai yellowwood, southern ochrosia), Ocimum spp. (Basil, Lemon basil, Sweet basil, tulsi), Odontonema spp. (Toothedthreads), Oenocarpus spp. (Turu palm, palma milpesos, bacaba, Patawa), Oenothera spp. (Primrose, evening primrose, suncup, sundrop), Olea spp. (Olive, black ironwood, ironwood, East African olive, Elgon teak), Onobrychis spp. (Sainfoin), Oplopanax spp. (Devil's club, Alaskan ginseng), Opuntia spp. (Prickly pear, tuna, nopal), Origanum spp. (Oregano, Marjoram, Cretan dittany, bible hyssop), Oryza spp. (Rice, wild rice, African rice, longstamen rice, red rice, Asian rice), Osmanthus spp. (Fragrant tea olive, Holly osmanthus), Osmunda spp. (Royal fern, flowering fern), Osmundastrum spp. (Cinnamon fern), Ostrya spp. (Hop-hornbeam, hophornbeam), Oxalis spp. (Wood sorrel, yellow sorrel, pink sorrel, false shamrock, Sourgrass, oxalise), Oxydendrum spp. (Sourwood, sorrel tree), Pachira spp. (Guiana chestnut, Money tree, Malabar chestnut, French peanut, Provision tree, Saba nut, Monguba, pochote), Pachyrhizus spp. (Jicama, yam bean, nupe, ahipa), Pachystachys spp. (Cardinals guard, lollipop plant, golden shrimp plant), Paeonia spp. (Peony, Polish Rose), Panax spp. (Ginseng, notoginseng, three-seven root, mountain plant, Pseudoginseng), Pandanus spp. (Pandan, screw palm, screw pine, Nicobar-breadfruit, Karuka), Pandorea spp. (Wonga vine, Bower of beauty, Pandoras vine, Boat vine), Panicum spp. (Panicgrass, Millet, panicum, witchgrass, tumbleweed, maidencane), Papaver spp. (Poppy), Parkia spp. (Bitter bean, African locust bean), Parkinsonia spp. (Palo verde, brea, verde olivo), Parrotia spp. (Persian ironwood, Chinese ironwood), Parthenocissus spp. (Virginia creeper, woodbine, sevenleaf creeper, Boston ivy), Passiflora spp. (Passionfruit, passion flowers, passion vine, Maypop, Granadilla), Pastinaca spp. (Parsnip), Paullinia spp. (Guarana, Yoco), Paulownia spp. (Pprincess tree, kiri, Korean paulownia, dragontree), Paxistima spp. (Oregon boxleaf, Canby's mountain-love), Pelargonium spp. (Geranium, storksbills, pelargonium), Peltophorum spp. (Weeping wattle, copperpod, yellow-flamboyant, yellow flametree, yellow poinciana), Pennisetum spp. (Fountaingrasses, pearl millet, kikuyu grass, feathertop grass), Penstemon spp. (Beardtongue), Pentalinon spp. (Wild Allamanda), Pentas spp. (Egyptian starcluster, Penta), Peperomia spp. (Radiator plant, Peperomia), Perilla spp. (Perilla, Japanese basil), Persea spp. (Avocado, Bay tree, Coyo, Redbay, Swampbay), Persicaria spp. (Waterpepper, knotweed, smartweed, hot mint), Petasites spp. (Butterbur, coltsfoots), Petroselinum spp. (Parsley), Petunia spp. (Petunia), Peucedanum spp. (Masterwort), Peumus spp. (Boldo), Phaseolus spp. (Bean, wild bean), Phellodendron spp. (Cork-tree, Phellodendron), Philadelphus spp. (Mock-orange), Philodendron spp. (Philodendron, rascagarganta, vilevine, treelover), Phlox spp. (Phlox, wild sweet william), Phoenix spp. (Date palm, Date), Pholiota spp. (Nameko, mushroom), Photinia spp. (Photinia), Phyllanthus spp. (Gooseberry, leafflower, scrubby spurge, red root floater, sand reverchonia, gripeweed, shatterstone), Phyllostachys spp. (Golden bamboo, fishpole bamboo, yellow groove bamboo, madake, timber bamboo, moso bamboo), Physalis spp. (Groundcherry, Tomatillo, husk tomatoes, Inca berry, poha berries, golden berries, Cape gooseberry), Physocarpus spp. (Ninebark), Picea spp. (Spruce), Pilea spp. (Aluminum Plant, Artillery Plant, silver sprinkles, friendship plant, creeping Charlie), Pimenta spp. (Allspice, bay rum tree, ciliment), Pimpinella spp. (Anise, aniseed, pimpinella, chamnamul, saxifrage), Pinckneya spp. (Georgia bark, Pinckneya), Pinus spp. (Pine), Piper spp. (Pepper, Pariparoba, Mexican pepperleaf, Betel vine), Pipturus spp. (Mamaki), Pistacia spp. (Pistachio, Mastic), Pisum spp (Pea), Pithecellobium spp. (Madras-thorn), Pittosporum spp. (Pittosporum, petroleum nut, cheesewood), Plantago spp. (Plantain), Platanus spp. (Planetree, Sycamores), Platonia spp. (Bacury), Platycladus spp. (Chinese arborvitae, biota), Platycodon spp. (Balloon flower), Plectranthus spp. (Spurflower), Pleurotus spp. (Oyster mushroom), Plinia spp. (Brazilian grapetree, jaboticaba, cambuce), Plumbago spp. (Plumbago, leadwort), Plumeria spp. (Plumeria, Frangipani), Podocarpus spp. (Yellowwood, Pine, Illawarra plum), Polygonatum spp. (Solomon's seal), Polypodium spp. (Polypodies, rockcap fern), Polyscias spp. (Ming aralia, 'ohe), Polystichum spp. (Fern), Poncirus spp. (Trifoliate orange), Pontederia spp. (Pickerel weeds), Populus spp. (Poplar, aspen, cottonwood), Porophyllum spp. (Coriander), Portulaca spp. (Purslane), Potentilla spp. (Cinquefoils, tormentils, barren strawberries), Pouteria spp. (Abiu, Canistel, LGcuma, Sapote), Primula spp. (Primrose), Proboscidea spp. (Unicorn plant), Prosopis spp. (Mesquite), Prostanthera spp. (Mintbush), Prunella spp. (Heal-all), Prunus spp. (Almond, Apricot, Cherry, Chokecherry, Nectarine, Peach, Plum, Plumcot, Prune, Sloe), Pseudanamomis spp. (Monos plum), Pseudolarix spp. (Golden larch), Pseudotsuga spp. (Douglas fir), Psidium spp. (Guava), Psychotria spp. (Wild Coffee), Ptelea spp. (Hoptrees), Pteridium spp. (Fern), Pterocarpus spp. (Saunders), Pterocarya spp. (Wingnuts), Pterostyrax spp. (Epaulette tree), Ptychosperma spp. (Cabbage Palm), Pueraria spp. (Kudzu), Punica spp. (Pomegranate), Pycnanthemum spp. (Mountainmint), Pyrostegia spp. (Flamevine), Pyrus spp. (Pear), Quararibea spp. (Guayabillo), Quassia spp. (Amargo), Quercus spp. (Oak), Quillaja spp. (Soapbark), Randia spp. (indigoberry), Raphanus spp. (Daikon, Radish), Raphia spp. (Wine palm), Ravenala spp. (Traveller's palm), Rehmannia spp. (Chinese foxglove), Rhamnus spp. (Buckthorn), Rhapis spp. (Lady palms), Rheum spp. (Rhubarb), Rhizophora spp. (True mangroves), Rhododendron spp. (Azalea, Labrador tea, Rhododendron), Rhus spp. (Sumac), Ribes spp. (Currant, Gooseberry, Jostaberry), Ricinodendron spp. (African nut), Ricinus spp. (Castor oil), Robinia spp. (Locust), Rorippa spp. (Cress), Rosa spp. (Rose), Rosemarinus spp. (Rosemary), Roystonea spp. (Royal palm), Rubus spp. (Raspberry, Blackberry, Cloudberry, Tayberry, Youngberry), Rudbeckia spp. (Coneflowers, black-eyed-susan), Ruellia spp. (Wild petunias), Rumex spp. (Sorrel, dock), Ruta spp. (Rue), Sabal spp. (Palmetto, Sabal), Sagittaria spp. (Wapato), Salacca spp. (Salak palm), Salix spp. (Willow), Salvia spp. (Sage, clary, rosemary, Chia), Sambucus spp. (Elderberry, Elder), Sandoricum spp. (Santol), Sanguisorba spp. (Burnet), Santalum spp. (Quandong), Sanvitalia spp. (Creeping zinnia), Sargassum spp. (Gulfweed, Sea Holly), Sassafras spp. (Sassafras), Satureja spp. (Savory), Savia spp. (Savia), Scabiosa spp. (Pincushion flowers), Scaevola spp. (Scaevolas, fan-flowers, half-flowers, naupaka, gullfeed), Schinus spp. (Peppertree), Schinziophyton spp. (Mongongo), Schisandra spp. (Magnolia berry), Schizonepeta spp. (Japanese catnip), Scolymus spp. (Golden thistle), Scorzonera spp. (Black salsify), Secale spp. (Rye), Sechium spp. (Chayote), Sedum spp. (Stonecrops), Senecio spp. (Ragworts, groundsels), Senegalia spp. (Gum arabic, Catechu), Senna spp. (Candlebush, Avarum), Sequoia spp. (Coastal redwood), Sequoiadendron spp. (Giant sequoia), Serenoa spp. (Saw palmetto), Sesamum spp. (Sesame), Sesuvium spp. (Sea-purslanes), Shepherdia spp. (Buffaloberry), Sidalcea spp. (Checkermallows), Silybum spp. (Milk thistle), Simarouba spp. (Simaruba), Simmondsia spp. (Jojoba), Sinapis ssp. (Mustard), Sisyrinchium spp. (Blue-eyed grasses), Sium spp. (Skirret, Water parsnips), Solanum spp. (Tomato, Potato, Cocona, Sunberry, Pepino, Naranjilla, Garden huckleberry, Eggplant), Solidago spp. (Goldenrod), Sophora spp. (Kowhai), Sorbus spp. (Mountain-ash, Serviceberry), Sorghastrum spp. (Indiangrass), Sorghum spp. (Sorghum), Spartina spp. (Cordgrass), Spathiphyllum spp. (Spath, peace lilies), Spathodea spp. (African tulip tree), Sphaeropteris spp. (Tree fern), Spinacia spp. (Spinach), Spiraea spp. (Spirea), Spondias spp. (Mombin), Stachys spp. (Betony, Hedgenettle), Stachytarpheta spp. (Porterweeds), Stenochlaena spp. (Fern), Sterculia spp. (Tropical chestnuts), Stevia spp. (Stevia), Stewartia spp. (Stewartia), Stokesia spp. (Stokes aster), Strelitzia spp. (Bird of Paradise), Stropharia spp. (Mushroom), Struthiopteris spp. (Deer fern), Styphnolobium spp. (Necklacepod), Styrax spp. (Snowbell), Suriana spp. (Bay cedar), Sutera spp. (Sutera), Swietenia spp. (Mahogany), Syagrus spp. (Overtop palm, licuri palm, queen palm), Symphoricarpos spp. (Snowberry), Synsepalum spp. (Miracle Fruit), Syringa spp. (Lilac), Syzygium spp. (Brush cherries, Waterberry, Clove), Tabebuia spp. (Trumpet tree), Tabernaemontana spp. (Milkwood), Tagetes spp. (Marigold), Talinum spp. (Fameflower), Tamarindus spp. (Tamarind), Tanacetum spp. (Tansy), Taraxacum spp. (Dandelion), Tasmannia spp. (Pepperbush), Taxodium spp. (Baldcypress, Pondcypress), Taxus spp. (Yew), Tecoma spp. (Trumpetbush), Tellima spp. (Fringecups), Terminalia spp. (Indian almond, Terminalia, Kakadu Plum), Ternstroemia spp. (Ternstroemia), Tetragonai spp. (Spinach), Tetrazygia spp. (Clover ash), Teucrium spp. (Germander), Theobroma spp. (Cacao), Thlaspi spp. (Pennycress), Thuja spp. (Arborvitaes, Thujas, Cedars), Thymus spp. (Thyme), Thyrsostachys spp. (Bamboo), Tiarella spp. (Foamflower), Tibouchina spp. (Tibouchina), Tilia spp. (Linden), Tolmiea spp. (Piggyback plant), Toona spp. (Redcedar), Torreya spp. (Nutmeg yew, Torreya), Trachycarpus spp. (Palm), Trachyspermum spp. (Ajowan), Tradescantia spp. (Spiderwort), Tragopogon spp. (Salsify), Tremella spp. (Fungus), Triadica spp. (Chinese tallowtree), Tribulus spp. (Caltrop), Tricholoma spp. (Fungus), Trientalis spp. (Starflowers), Trifolium spp. (Clover), Trigonella spp. (Fenugreek), Trillium spp. (Trillium), Triticum spp. (Wheat), Tropaeolum spp. (Nasturtium), Tsuga spp. (Hemlock Tree), Tuber spp. (Truffle), Turbinaria spp. (Disc Coral, Scroll Coral, Cup Coral, Vase Coral, Pagoda Coral, Ruffled Ridge Coral), Turnera spp. (Damiana), Typha spp. (Cattail, bulrush, reedmace, reed, punks, raupo), Uapaca spp. (Sugar plum), Ugni spp. (Chilean guava), Ulmus spp. (Elm), Uncaria spp. (Cat's claw, Gambir), Ungnadia spp. (Mexican buckeye), Uniola spp. (Sea Oats), Urtica spp. (Nettle), Vaccinium spp. (Blueberry, Cranberry, Huckleberry, Lingonberry), Valerianella spp. (Corn salad), Vancouveria spp. (Inside-out flowers), Vangueria spp. (Spanish-tamarind), Vanilla spp. (Vanilla), Vasconcellea spp. (Mountain Papaya, Babaco), Verbascum spp. (Mullein), Verbena spp. (Verbena), Vernonia spp. (Ironweed), Veronica spp. (Speedwell), Viburnum spp. (Cranberry, Viburnum), Vicia spp. (Vetch), Vigna spp. (Bean), Viola spp. (Pansy, Violet), Vitex spp. (Plum, Chastetree), Vitis spp. (Grape), Volvariella spp. (Mushroom), Washingtonia spp. (Palm), Wedelia spp. (Creeping-oxeye), Wisteria spp. (Wisteria), Withania spp. (Ashwagandha), Xanthoceras spp. (Yellowhorn), Xanthosoma spp. (Tanier), Ximenia spp. (Tallowwood), Xylopia spp. (Grains of Selim), Yucca spp. (Yucca), Zamia spp. (Cycad), Zanthoxylum spp. (Pepper), Zea spp. (Corn, Teosinte), Zelkova spp. (Zelkova), Zephyranthes spp. (Lily), Zingiber spp. (Ginger), Zinnia spp. (Zinnia), Zizania spp. (Wild Rice), and/or Ziziphus spp. (Jujube, Zizafun).

In some embodiments, a plant useful with this invention includes but is not limited to those listed in Table 2 or Table 4 or the list provided in the above paragraph. In some embodiments, example plants useful with this invention include a citrus plant (e.g., grapefruit, orange, lemon, lime and the like), a tomato plant, a corn plant, a pecan plant, and a tobacco plant.

TABLE 1
Plast proteins

| | Accession | |
|---|---|---|
| Plast protein | number | Bacterial origin |
| 15834-N- | ABI15642.1 | Agrobacterium rhizogenes |
| IaaM | | |
| 1724-Orf13 | BAA22337.1 | Agrobacterium rhizogenes |
| 1724-Orf14 | BAA22339.1 | Agrobacterium rhizogenes |
| 2659-Orf14 | CAB65899.1 | Agrobacterium rhizogenes |
| 2659-RoIB | CAA82552.1 | Agrobacterium rhizogenes |
| 2659-RoIC | CAA82553.1 | Agrobacterium rhizogenes |
| 8196-Orf13 | AAA22097.1 | Agrobacterium rhizogenes |
| 8196-Orf14 | AAA22099.1 | Agrobacterium rhizogenes |
| 8196-RoIC | AAA22096.1 | Agrobacterium rhizogenes |
| A4-N-Orf8 | ABI54188.1 | Agrobacterium rhizogenes |
| A4-Orf13 | ABI54192.1 | Agrobacterium rhizogenes |
| A4-Orf14 | ABI54193.1 | Agrobacterium rhizogenes |
| A4-RoIBTR | CAA34077.1 | Agrobacterium rhizogenes |
| A4-RoIC | P20403.1 | Agrobacterium rhizogenes |
| K599-N- | ABS11822.1 | Agrobacterium rhizogenes |
| Orf8 | | |
| 15955-N- | CAA25167.1 | Agrobacterium tumefaciens |
| IaaM | | |
| 15955-p4′ | CAA25180.1 | Agrobacterium tumefaciens |
| Ach5-6a | P04030.1 | Agrobacterium tumefaciens |
| Bo542-6b | AAA98501.1 | Agrobacterium tumefaciens |
| Bo542-d | AAZ50418.1 | Agrobacterium tumefaciens |
| Bo542-p4′ | AAZ50416.1 | Agrobacterium tumefaciens |
| Bo542-p5 | AAZ50393.1 | Agrobacterium tumefaciens |
| Bo542-p7 | AAZ50396.1 | Agrobacterium tumefaciens |
| C58-6a | AAK90971.1 | Agrobacterium tumefaciens |
| C58-6b | AAK90972.1 | Agrobacterium tumefaciens |
| C58-b | AAD30482.1 | Agrobacterium tumefaciens |
| C58-c′ | AAD30484.1 | Agrobacterium tumefaciens |
| C58-d | AAD30485.1 | Agrobacterium tumefaciens |
| C58-N-IaaM | CAB44640.1 | Agrobacterium tumefaciens |
| C58-p5 | AAD30487.1 | Agrobacterium tumefaciens |
| Chry5-6b | AAB49454.1 | Agrobacterium tumefaciens |
| Chry-e | AAK08598.1 | Agrobacterium tumefaciens |
| Lso | AAC25913.1 | Agrobacterium tumefaciens |
| oct-p3′ | CAA25183.1 | Agrobacterium tumefaciens |
| oct-p7 | AAF77121.1 | Agrobacterium tumefaciens |
| SAK-e | BAA87804.1 | Agrobacterium tumefaciens |
| t-Orf14 | CBJ56561.1 | Agrobacterium tumefaciens |
| AB4-6b | CAA54541.1 | Agrobacterium vitis |
| AB4-p3′ | CAA54542.1 | Agrobacterium vitis |
| Agl62-N- | AAC77909.1 | Agrobacterium vitis |
| IaaM | | |
| CG474-6b | AAB41871.1 | Agrobacterium vitis |
| CG474-p5 | AAB41867.1 | Agrobacterium vitis |
| NCPPB3554- | KWT91792.1 | Agrobacterium vitis |
| 6a | | |
| S4-6b | AAA25043.1 | Agrobacterium vitis |
| S4-N-IaaM | AAA98149.1 | Agrobacterium vitis |
| Tm4-6b | CAA39648.1 | Agrobacterium vitis |
| Tm4-p5 | AAB41873.1 | Agrobacterium vitis |
| Tm4-TA- | P25017.1 | Agrobacterium vitis |
| N-IaaM | | |
| Tm4-TB-b | AAD30490.1 | Agrobacterium vitis |
| Tm4-TB- | AAD30493.1 | Agrobacterium vitis |
| N-IaaM | | |

TABLE 2
Example plants that are natural hosts for
Agrobacterium spp. or in which Agrobacterium spp.
have been used in DNA transfer processes

| Common Name | Scientific Name |
|---|---|
| Alfalfa | Medicago sativa |
| Almond | Prunus dulcis |
| Amaranth | Amaranthus mangostanus |
| Antler shape | L. sativa var. crispa |
| leaf lettuce | |
| Apiaceae Carrot | Daucus carota |
| Apple | Malus domestica |
| Apricot | Prunus armeniaca |
| Asparagus | Asparagus officinalis |
| Asparagus bean | V. unguiculata |
| Aubergine | Solanum melongena |
| | var. esculentum |
| Azuki bean | Vigna angularis |
| Balsam | Impatiens walleriana |
| Bamboo | Dendrocalamus latiflorus |
| Banana | Musa acuminate |
| Barley | Hordeum vulgare |
| Bean | Phaseolus vulgaris |
| Bell pepper | Capsicum annuum |
| Bentgrass | Agrostis palustris |
| Bermudagrass | Cynodon dactylon |
| Bitter gourd | Momordica charantia cv. 2486 |
| Blackberry | Rubus ursinus |
| Blueberry | Vaccinium corymbosum |
| Bluegrass, | Poa pratensis |
| Kentucky | |
| Bottle gourd | Lagenaria sciceraria |
| | cv. Chun-Yin |
| Broccoli | Brassica oleracea var. italica |
| Brown mustard | Brassica juncea var. crispifolia |
| Cabbage | B. oleracea var. capitata |
| Cannabis | Cannabis sativa |
| Carnation | Dianthus caryophyllus |
| Carrot | Daucus carota |
| Cauliflower | Brassica oleracea |
| Celery | Apium graveolens |
| Cherry | Prunus avium |
| Chestnut | Castanea dentata |
| (American) | |
| Chicory | Cichorium intybus |
| Chinese amaranth | Amaranthus tricolor |
| Chinese cabbage | Brassica campestris |
| | pekinensis |
| Ching chiang | Brassica chinensis |
| pai-tsai | |
| Cilantro | Coriadium sativum |
| Climbing spinach | Basella rubra |
| Coconut | Cocos nucifera |
| Coffee | Coffea arabica |
| Coffee | Coffea canephora |
| Corn | Zea mays |
| Cotton | Gossypium hirsutum |
| Cowpea | Vigna unguiculata |
| | cv. 131 Farmers |
| Cowpea | V. unguiculata cv. Bai-Pi |
| Cowpea | V. unguiculata cv. Purple mart |
| Cowpea | V. unguiculata cv. 101 Farmers |
| Cowpea | V. unguiculata cv. |
| | Green pod Kaohsiung |
| Cowpea | V. unguiculata cv. Bai-He |
| Cucumber | Cucumis sativus |
| Douglas Fir | Pseudotsuga menziesii |
| Duckweed | Lemna minor |
| Eucalyptus | Eucalyptus camaldulensis |
| Garden cosmos | Cosmos bipinnatus |
| Garlic | Allium sativum |
| Grapefruit | Citrus paradisis |
| Green pepper | Capsicum annuum |
| Hazelnut | Corylus avellane |
| Head mustard | B. juncea var. capitata |
| Kidney bean | Phaseolus vulgaris |
| Kiwifruit | Actinidia deliciosa |
| Lemon | Citrus limon |
| Lentil | Lens culinaris |
| Lettuce | Lactuca sativa |
| Lime | Citrus aurantifolia |
| Lima bean | Phaseolus lunatus |
| Loblolly pine | Pinus taeda |
| Loose leaf lettuce | L. sativa var. crispa |
| Luffa | Luffa cylindrical cv. Mei-Ren |
| Macadamia | Macadamia integrifolia |
| Mealy sage | Salvia farinacea |
| Melons | Cucumis melo |
| Mung bean | Vigna radiate |
| Oats | Avena sativa |
| Oil palm | Elaeis guineensis |
| Oilseed rape | Brassica napus, |
| | B. oleraceae, B. juncea |
| Olive | Olea europaea |
| Onion | Allium cepa |
| Orange | Citrus sinensis |
| Pai-tsai | Brassica rapa. var. chinensis |
| Pea | Pisum sativum |
| Peach | Prunus persica |
| Pear | Pyrus communis |
| Pearl millet | Pennisetum glaucum |
| Pecan | Carya illinoinensis |
| Pigeon pea | Cajanus cajan |
| Pineapple | Ananus comosus |
| Plum | Prunus domestica |
| Plumed cockscomb | Celosia argentea var. plumosa |
| Poinsettia | Euphorbia pulcherriuma |
| Pomegranate | Punica granatum |
| Pondersa pine | Pinus ponderosa |
| Potato | Solarium tuberosum |
| Pumpkin | Cucurbita pepo |
| Raspberry (black) | Rubus occidentalis |
| Raspberry (red) | Rubusidaeus |
| Rice | Oryza spp |
| Romaine lettuce | L. sativa var. romana |
| Rose | Rosa hybrida |
| Rye | Secale cereal |
| Ryegrass | Lolium rigidum |
| Scallions | Allium fistulosum |
| Snap bean | P. vulgaris cv. Taichung No. 3 |
| Snapdragon | Antirrhinum majus |
| Sorghum | Sorghum bicolor |
| Soybean | Glycine max cv. |
| | Chin-Ren-WooDow, CRWD |
| Soybean | G. max cv. Tainan No. 7 |
| Soybean | G. max cv. Gao-Gai No. 5 |
| Soybean | G. max cv. Kaohsiung No. 5 |
| Spinach | Spinacia oleracea |
| Squash | Cucurbita moschata |
| Squash | Cucurbita maxima |
| St. Augustinegrass | Stenotaphrum secundatum |
| Strawberry | Fragaria grandiflora |
| Sugarbeet | Beta vlugaris |
| Sugarcane | Saccharum officinarum |
| Sweet alyssum | Lobularia maritima |
| Sweet basil | Ocimum basilicum |
| Sweet pea | Lathyrus odoratus |
| Sweet potato | Ipomoea batatas |
| Tall fescue | Festuca arundinacea |
| Thale cress | Arabidopsis thaliana |
| Tobacco | Nicotiana tabacum |
| Tomato | Solanum lycopersicum |
| Vinca | Catharanthus roseus |
| Walnut (English) | Juglans regia |
| Watermelon | Citrullus lanatus |
| Water spinach | Ipomoea aguatica |
| Wheat | Triticum aestivum |
| White dutch | Phaseolus coccineus |
| runner bean | var. albonanus |
| White leaf lettuce | Lactuca sativa var. white leaf |
| White radish | Raphanus sativus |
| Yam | Dioscorea rotundata |
| Zoysiagrass | Zoysia japonica |

TABLE 3
Example stylet sheath inhibitory peptides

| | Enzyme Commission |
|---|---|
| Protease | Number |
| Amylase | EC # : 232-588-1 |
| Amyloglucosidase | EC # : 232-877-2 |
| Ficin | EC # : 232-599-1 |
| Carboxypeptidase W | EC # : 3.4.16.6 |
| Chymopapain | EC # : 232-580-8 |
| Papain | EC # : 232-627-2 |
| Bromelain | EC # : 253-387-5 |
| Trypsin | EC # : 232-650-8 |
| Collagenase Type VII | EC # : 232-582-9 |
| Laminarinase | EC # : 3.2.1.6 |
| Licheninase | EC # : 3.2.1.73 |
| Beta (1-3)-D-Glucanase | EC # : 232-927-3 |
| Proteinase K | EC # : 3.4.21.64 |

TABLE 4
Example plants and diseases and pests of the same

| | | Common | |
|---|---|---|---|
| Genus | Species | name | Target Pests or Diseases |
| Actinidia | deliciosa | Kiwifruit | Phytophthora root rot (several |
| | | | Phytophthora spp.), gray mold |
| | | | (Botrytis cinerea), bacterial |
| | | | blossom blight (Pseudomonas |
| | | | viridiflava), bleeding canker |
| | | | (Pseudomonas syringae), oak |
| | | | root fungus (Armillaria mellea), |
| | | | omnivorous leafroller |
| | | | (Platynota stultana) |
| Allium | cepa | Onion | Downy mildew (Peronospora |
| Allium | sativum | Garlic | destructor), purple blotch |
| Allium | porrum | Leek | (Alternaria porri, gray mold |
| | | | (Botrytis spp.), thrips (Thrips |
| | | | tabaci and Frankliniella |
| | | | occidentalis), mites (Rhizoglyphus |
| | | | spp.), lesion nematode |
| | | | (Pratylenchus penetrans) |
| Apium | graveolens | Celery | Bacterial blight (Pseudomonas |
| | | | cichorii), soft rot (Erwinia |
| | | | carotovora, Erwinia |
| | | | chrysanthemi, Pseudomonas |
| | | | marginalis), damping off |
| | | | (Pythium spp., Rhizoctonia |
| | | | solani), downy mildew |
| | | | (Peronospora umbellifarum), |
| | | | early blight (Cercospora apii), |
| | | | aphids (Myzus persicae, |
| | | | Aphis gossypii), armyworm |
| | | | (Pseudaletia unipuncta), |
| | | | root knot nematode |
| | | | (Meloidogyne spp.) |
| Aspara- | officinalis | Aspara- | Asparagus rust (Puccinia |
| gus | | gus | asparagi), cercospora blight |
| | | | (Cercospora asparagi), |
| | | | Fusarium wilt (Fusarium |
| | | | oxysporum), Phytophthora |
| | | | rot (Phytophthora spp.), |
| | | | asparagus beetle (Crioceris |
| | | | asparagi, Crioceris |
| | | | duodecimpunctata) |
| Avena | sativa | Oats | Crown rust (Puccinia |
| | | | coronata), powdery mildew |
| | | | (Erysiphe graminis), aphids |
| | | | (Diuraphis noxia, Sitobion |
| | | | avenae, Rhopalosuphum padi) |
| Beta | vulgaris | Sugar | Bacterial blight (Pseudomonas |
| | | beet | syringae), beet curly top |
| | | | disease (beet curly top virus, |
| | | | beet severe curly top virus, |
| | | | beet mild curly top virus), |
| | | | cercospora leaf spot |
| | | | (Cercospora beticola), downy |
| | | | mildew (Peronospora |
| | | | farinosa), powdery mildew |
| | | | (Erysiphe betae), beet cyst |
| | | | nematode (Heterodera |
| | | | schachtii), root knot |
| | | | nematode (Meloidogyne |
| | | | spp.), leafminers |
| | | | (Lyriomyza spp.) |
| Brassica | oleracea | Broccoli | Alternaria leaf spot |
| | | | (Alternaria brassicae), black rot |
| | | | (Xanthomonas campestris), |
| | | | clubroot (Plamodiophora |
| | | | brassicae), powdery mildew |
| | | | (Erysiphe cruciferarum), |
| | | | Sclerotinia stem rot |
| | | | (Sclerotinia sclerotiorum), |
| | | | blackleg (Phoma lingam), |
| | | | downy mildew |
| | | | (Hyaloperonospora parasitica), |
| | | | diamondback moth |
| | | | (Plutella xylostella), flea |
| | | | beetle (Phyllotreta cruciferae), |
| | | | cabbageworm (Pieris rapae), |
| | | | thrips (Frankliniella |
| | | | occidentalis, Thrips tabaci), |
| | | | root knot |
| | | | nematode (Meloidogyne spp.) |
| | oleracea | Cauli- | Bacteria soft rot (Erwinia |
| | | flower | caratovora), blackleg |
| | | | (Leptosphaeria maculans), |
| | | | black rot (Xanthomonas |
| | | | campestris), clubroot |
| | | | (Plsamodiophora brassicae), |
| | | | downy mildew |
| | | | (Hyaloperonospora parasitica), |
| | | | powdery mildew (Erysiphe |
| | | | cruciferarum), sclerotinia |
| | | | stem rot (Sclerotinia |
| | | | sclerotiorum), army worm |
| | | | (Spodoptera exigua), |
| | | | cabbage aphid (Brevicorne |
| | | | brassicaea), cabbage looper |
| | | | (Trichoplusia ni), cucumber |
| | | | beetles (Diaboritica |
| | | | undecimpunctata), |
| | | | diamondback moth (Plutella |
| | | | xylostella), flea beetle |
| | | | (Phyllotreta cruciferae), |
| | | | cabbageworm (Pieris rapae), |
| | | | thrips (Frankliniella occidentalis, |
| | | | Thrips tabaci), root knot |
| | | | nematode (Meloidogyne spp.) |
| | oleracea | Cabbage | Alternaria leaf spot |
| | | | (Alternaria brassicae), |
| | | | anthracnose (Colletotrichum |
| | | | higginsianum) black rot |
| | | | (Xanthomonas campestris), |
| | | | clubroot (Plamodiophora |
| | | | brassicae), powdery |
| | | | mildew (Erysiphe cruciferarum), |
| | | | Sclerotinia stem rot |
| | | | (Sclerotinia sclerotiorum), |
| | | | bacterial soft rot (Erwinia |
| | | | caratovora), downy mildew |
| | | | (Peronospora parasitica), |
| | | | beet armyworm |
| | | | (Spodoptera exigua), cabbage |
| | | | aphid (Brevicoryne brassicaea), |
| | | | cabbage looper (Trichoplusia ni), |
| | | | diamondback moth (Plutella |
| | | | xylostella), flea beetle |
| | | | (Phyllotreta cruciferae), |
| | | | cabbageworm (Pieris rapae), |
| | | | thrips (Frankliniella occidentalis, |
| | | | Thrips tabaci), root |
| | | | knot nematode (Meloidogyne spp.) |
| | napus | Rapeseed | Alternaria leaf spot (Alternaria |
| | | | brassicae), black rot |
| | | | (Xanthomonas campestris), |
| | | | downy mildew (Peronospora |
| | | | parasitica), Sclerotinia stem rot |
| | | | (Sclerotinia sclerotiorum), |
| | | | blackleg (Leptosphaeria |
| | | | maculans), cabbage aphid |
| | | | (Brevicoryne brassicaea), |
| | | | flea beetle (Phyllotreta cruciferae) |
| Cajanus | cajan | Pigeon | Alternaria blight (Alternaria |
| | | pea | alternata), anthracnose |
| | | | (Colletotrichum spp.), cercospora |
| | | | leaf spot (Cercospora cajani), |
| | | | white mold (Sclerotinia |
| | | | sclerotiorum), aphids (Aphis |
| | | | craccivora), armyworms |
| | | | (Spodoptera |
| | | | exigua, S. praefica), corn earworm |
| | | | (Helicoverpa zea), leafminers |
| | | | (Lyriomyza spp.) |
| Cannabis | sativa | Cannabis | Gray mold (Botrytis cinerea), |
| | | | powdery mildew (Golovinomyces |
| | | | cichoracearum), damping off |
| | | | (Pythium spp.), spider mites |
| | | | (Tetranychus urticae) |
| Capsicum | annuum | Bell | Anthracnose (Colletotrichum |
| | | pepper, | spp.), cercospora leaf |
| | | chile | spot (Cercospora capsici), |
| | | pepper, | fusarium wilt (Fusarium |
| | | Jalapeno | oxysporum), powdery |
| | | pepper, | mildew (Leveillula taurica), |
| | | etc. | southern blight (Sclerotium |
| | | | rolfsii), verticillium wilt |
| | | | (Verticillium spp.), bacteria |
| | | | canker (Calvibacter |
| | | | michiganensis), bacterial |
| | | | spot (Xanthomonas spp.), |
| | | | Phytophthora blight |
| | | | (Phytophthora caspsici), aphids |
| | | | (Myzus persicae), armyworm |
| | | | (Spodoptera exigua), Colorado |
| | | | potato beetle (Leptinotarsa |
| | | | decemlineata), leafminer (Lyriomyza |
| | | | spp.), leafroller (Platynota |
| | | | stultana), pepper weevil |
| | | | (Anthonomus eugenii), thrips |
| | | | (Frankliniella occidentalis, |
| | | | Thrips tabaci), tomato |
| | | | fruitworm (Helicoverpa zea), |
| | | | spider mites |
| | | | (Tetranychus urticae) |
| Carya | illinoi- | Pecan | Anthracnose (Colletotrichum |
| | nensis | | gloeosporoides), downy spot |
| | | | (Pseudocercosporella |
| | | | caryigena), powdery mildew |
| | | | (Phyllactinia guttata), bacterial |
| | | | leaf scorch (Xylella fastidiosa), |
| | | | scab (Cladosporium caryigenum), |
| | | | shuck & kernel rot |
| | | | (Phytophthora cactorum), black |
| | | | pecan aphid (Melanocallis |
| | | | caryaefollae), pecan nut |
| | | | caseborer (Acrobasis |
| | | | nuxvorella), pecan weevil |
| | | | (Curculio caryae) |
| Castanea | dentata | American | Blight (Cryphonectria parasitica), |
| | | chestnut | chestnut weevil (Curculio |
| | | | sayi, C. caryatrypes), Japanese |
| | | | beetle (Popillia japonica) |
| Citruilus | lanatus | Water- | Alternaria leaf blight |
| | | melon | (Alternaria cucumerina), |
| | | | anthracnose (Colletotrichum |
| | | | orbiculare), cercospora leaf |
| | | | spot (Cercospora citrullina), |
| | | | downy mildew |
| | | | (Pseudoperonospora |
| | | | cubensis), fusarium wilt |
| | | | (Fusarium oxysporum), |
| | | | gummy stem blight |
| | | | (Didymella bryoniae), |
| | | | powdery mildew (Podosphaera |
| | | | xanthii, P. fuliginea), |
| | | | verticillium wilt (Verticillium |
| | | | dahliae), angular leaf spot |
| | | | (Pseudomonas syringae), aphids |
| | | | (Myzus persicae, Aphid |
| | | | gossypii), cabbage looper |
| | | | (Trichoplusia ni), flea |
| | | | beetles (Epitrix spp.), |
| | | | thrips (Frankliniella |
| | | | occidentalis, Thrips tabaci) |
| Citrus | paradisis | Grapefruit | Anthracnose (Colletotrichum |
| | limon | Lemon | gloeosporioides), canker |
| | auran- | Lime | (Xanthomonas axonopodis), |
| | tiifolia | Orange | HLB (Candidates Liberibacter |
| | sinensis | | asiaticus), melanose (Diaporthe |
| | | | citri), tristeza (Citrus tristeza |
| | | | virus), Asian citrus psyllid |
| | | | (Diaphorina citri), black |
| | | | citrus aphid (Toxoptera |
| | | | aurantii), citrus leaf miner |
| | | | (Phyllocnistis citrella), thrips |
| | | | (Scirtothrips citri), brown |
| | | | marmorated stinkbug |
| | | | (Halyomorpha halys) |
| Cocos | nucifera | Coconut | Bud rot & nutfall (Phytophthora |
| | | | spp., Fusarium solani, |
| | | | F. moniliforme, Graphium |
| | | | spp.), coconut bug |
| | | | (Pseudotheraptus wayi), |
| | | | coconut rhinoceros beetle |
| | | | (Oryctes rhinoceros), |
| | | | mealybugs (Dysmicoccus brevipes, |
| | | | Ferisia virgata, Pianococcus |
| | | | lilacinus), red ring nematode |
| | | | (Bursaphelenchus cocophilus) |
| Coffea | arabica | Coffee | Bacterial blight (Pseudomonas |
| | canephora | Coffee | syringae), cercospora leaf spot |
| | | | (Cercospora coffeicola), |
| | | | coffee berry disease |
| | | | (Colletotrichum kahawae), |
| | | | coffee leaf rust (Hemileia vastatrix), |
| | | | black twig borer (Xylosandrus |
| | | | compactus), coffee berry borer |
| | | | (Hypothenemus hampei) |
| Corylus | avellana | Hazelnut | Armillaria root rot (Armillaria |
| | | | mellea), eastern filbert |
| | | | blight (Anisogramma anomalae), |
| | | | powdery mildew |
| | | | (Phyllactinia guttata), |
| | | | bacterial blight (Xanthomonas |
| | | | campestris), bacterial canker |
| | | | (Pseudomonas syringae), |
| | | | filbertworm |
| | | | (Cydia latiferreana), nut weevil |
| | | | (Curculio occidentis) |
| Cucumis | melo | Canta- | Alternaria leaf blight (Alternaria |
| | | loupe, | cucumerina), anthracnose |
| | | honey- | (Colletotrichum orbiculare), |
| | | dew, | cercospora leaf spot (Cercospora |
| | | musk- | citrullina), fusarium wilt (Fusarium |
| | sativus | Cucumber | oxysporum), gummy stem blight |
| | | | (Didymella bryoniae), powdery |
| | | | mildew (Podosphaera xanthii, |
| | | | Erysiphe cichoracearum), |
| | | | septoria leaf spot (Septoria |
| | | | cucurbitacearum), southern blight |
| | | | (Sclerotium rolfsii), verticillium |
| | | | wilt (Verticillium dahliae), angular |
| | | | leaf spot (Pseudomonas syringae), |
| | | | bacterial wilt (Erwinia |
| | | | tracheiphila), downy mildew |
| | | | (Pseudoperonospora cubensis), |
| | | | aphids (Myzus persicae, Aphis |
| | | | gossypii), cabbage looper |
| | | | (Trichoplusia ni), cucumber |
| | | | beetles (Acalymma vittata, |
| | | | Diabrotica undecimpunctata, |
| | | | D. balteata), flea beetles (Epitrix |
| | | | spp.), spotted wing drosophila |
| | | | (Drosophila suzukii), squash |
| | | | bug (Anasa tristis), thrips |
| | | | (Frankliniella occidentalis), |
| | | | root knot nematode |
| | | | (Meloidogyne spp.) |
| Cucurbita | pepo | Pumpkin | Alternaria leaf blight (Alternaria |
| | moschata | “Butter- | cucumerina), Alternaria leaf spot |
| | | nut” | (Alternaria alternata), cercospora |
| | | squash | leaf spot (Cercospora citrullina), |
| | maxima | Squash | downy mildew (Pseudoperonospora |
| | | | cubensis), gummy stem blight |
| | | | (Didymella bryoniae), powdery |
| | | | mildew (Erysiphe cichoracearum, |
| | | | Sphaerotheca fuliginea, |
| | | | Podosphaera xanthii), |
| | | | septoria leaf spot (Septoria |
| | | | cucurbitacearum), verticillium |
| | | | wilt (Verticillium dahliae), angular |
| | | | leaf spot (Pseudomonas syringae), |
| | | | bacteria leaf spot (Xanthomonas |
| | | | campestris), crown and root rot |
| | | | (Phytophthora capsici), aphids |
| | | | (Myzus persicae, Aphis |
| | | | gossypii), armyworms (Spodoptera |
| | | | exigua, S. praefica), cabbage |
| | | | looper (Trichoplusia ni), |
| | | | cucumber beetles (Acalymma |
| | | | vittata, Diabrotica |
| | | | undecimpunctata, D. balteata), |
| | | | flea beetles (Epitrix spp.), |
| | | | leafminers (Lyriomyza spp.), |
| | | | squash bug (Anasa tristis), |
| | | | squash vine borer (Melittia |
| | | | cucurbitae), thrips (Frankliniella |
| | | | occidentalis) |
| Daucus | carota | Carrot | Alternaria leaf blight (Alternaria |
| | | | dauci), black rot (Alternaria |
| | | | radicina), cercospora leaf blight |
| | | | (Cercospora carotae), downy |
| | | | mildew (Peronospora |
| | | | umbellifarum), powdery |
| | | | mildew (Erysiphe heraclei), |
| | | | bacterial leaf blight (Xanthomonas |
| | | | campestris), soft rot (Erwinia |
| | | | carotovora), aphids (Cavariella |
| | | | aegopodii), carrot weevil |
| | | | (Listronotus oregonensis), |
| | | | flea beetle (Systena blanda), |
| | | | root knot nematodes |
| | | | (Meloidogyne spp.) |
| Euca- | camal- | Euca- | Armillaria root rot (Armillaria |
| lyptus | dulensis | lyptus | mellea), canker (Botyrosphaeria |
| | | spp. | spp.), leaf spot (Mycosphaerella |
| | | | nubilosa) Eucalyptus snout |
| | | | beetle (Gonipterus scutellatus), |
| | | | redgum lerp psyllid (Glycaspis |
| | | | brimblecombei) |
| Fragaria | grandiflora | Straw- | Angular leaf spot (Xanthomonas |
| | | berry | fragariae), leaf scorch (Diplocarpon |
| | | | earlianum), anthracnose |
| | | | (Colletotrichum fragariae), gray |
| | | | mold (Botrytis cinerea), powdery |
| | | | mildew (Sphaerotheca macularis), |
| | | | aphids (Myzus persicae, |
| | | | Macrosiphon euphorbiae, Aphis |
| | | | gossypii), armyworm (Spodoptera |
| | | | exigua, S. eridania), loopers |
| | | | (Trichoplusia ni), thrips |
| | | | (Frankliniella occidentalis), |
| | | | weevils (Otiorhynchus spp.) |
| Glycine | max | Soybean | Rust (Phakopsora pachyrhizi), |
| | | | sclerotinia stem rot |
| | | | (Sclerotinia sclerotiorum), |
| | | | armyworms (Spodoptera exigua, |
| | | | S. praefica), cucumber beetles |
| | | | (Acalymma vittata, Diabrotica |
| | | | undecimpunctata), Mexican been |
| | | | beetles (Epilachna varivestis) |
| Gossy- | hirsutum | Cotton | Alternaria leaf spot (Alternaria |
| pium | | | macrospora), cercospora leaf |
| | | | spot (Cercospora gossypina), |
| | | | fusarium wilt (Fusarium |
| | | | oxysporum), target spot |
| | | | (Corynespora cassiicola), |
| | | | aphids (Aphis gossypii), |
| | | | armyworm (Spodoptera exigua), |
| | | | cotton bollworm |
| | | | (Helicoverpa zea) |
| Hordeum | vulgare | Barley | Fusarium head blight (Fusarium |
| | | | graminearum), net |
| | | | blotch (Pyrenophora teres), |
| | | | powdery mildew (Blumeria |
| | | | graminis), barley yellow dwarf |
| | | | (barley yellow dwarf virus), |
| | | | Russian wheat aphid (Diuraphis |
| | | | noxia), armyworms (Mythimna |
| | | | unipunctata, Spodoptera |
| | | | praefica), barley mealybug |
| | | | (Trionymus haancheni), |
| | | | stinkbugs (Euschistus spp.) |
| Ipomoea | batatas | Sweet | Alternaria leaf spot (Alternaria |
| | | potato | spp.), bacterial soft |
| | | | rot (Erwinia chrysanthemi), |
| | | | bacterial wilt (Ralstonia |
| | | | solanacearum), leaf & stem scab |
| | | | (Sphaceloma batatas), white |
| | | | grubs (Phyllophaga ephilida) |
| Juglans | regia | English | Anthracnose (Gnomonia |
| | | walnut | leptostyla), Armillaria root |
| | | | rot (Armillaria mellea), powdery |
| | | | mildew (Phyllactinia guttata), |
| | | | walnut blight (Xanthomonas |
| | | | campestris), phytophthora |
| | | | root rot (Phytophthora spp.), |
| Lactuca | sativa | Lettuce | Leaf drop (Sclerotinia minor), |
| | | | powdery mildew (Erysiphe |
| | | | cichoracearum), downy mildew |
| | | | (Bremia lactucae), armyworm |
| | | | (Mythimna unipuncta), beet |
| | | | armyworm (Spodoptera exigua), |
| | | | leafminers (Liriomyza spp.), |
| | | | cabbage looper (Trichoplusia ni), |
| | | | Lygus bug (Lygus hesperus), thrips |
| | | | (Frankliniella occidentalis) |
| Lens | culinaris | Lentil | Anthracnose (Colletotrichum |
| | | | truncatum), powdery |
| | | | mildew (yErysiphe pisi), |
| | | | gray mold (Botrytis |
| | | | cinerea), sclerotinia rot |
| | | | (Sclerotinia rolfsii), aphids |
| | | | (Acyrthosiphon pisum), |
| | | | Lygus bug (Lygus lineolaris) |
| Lyco- | esculentum | Tomato | Anthracnose (Colletotrichum |
| persicum | | | coccodes), black mold |
| | | | (Alternaria alternata), early blight |
| | | | (Alternaria solani), late |
| | | | blight (Phytophthora infestans), |
| | | | gray mold (Botrytis |
| | | | cinerea), septoria leaf spot |
| | | | (Septoria lycopersici), target |
| | | | spot (Corynespora cassiicola), |
| | | | verticillium wilt (Verticillium |
| | | | dahliae), bacterial canker |
| | | | (Clavibacter michiganensis), |
| | | | bacterial speck (Pseudomonas |
| | | | syringae), bacterial spot |
| | | | (Xanthomonas campestris, |
| | | | X. vesicatoria), bacterial |
| | | | wilt (Raistonia solanacearum), |
| | | | tomato mosaic virus |
| | | | (ToMV), aphids (Myzus persicae, |
| | | | Macrosiphon euphorbiae), beet |
| | | | armyworm (Spodoptera exigua), |
| | | | Colorado potato beetle |
| | | | (Leptinotarsa decemlineata), |
| | | | flew beetles (Epitrix |
| | | | spp.), hornworms (Manduca |
| | | | sexta, M. quinquemaculata), |
| | | | leafminers (Tuta absoluta, |
| | | | Liriomyza spp.), loopers |
| | | | (Trichoplusia ni, Autographa |
| | | | californica), thrips (Frankliniella |
| | | | occidentalis, Thrips tabaci), tomato |
| | | | fruitworm (Helicoverpa zea), |
| | | | spotted wing drosophila |
| | | | (Drosophila suzukii), root knot |
| | | | nematode (Meloidogyne spp.), |
| | | | spider mites (Tetranychus |
| | | | urticae), brown marmorated |
| | | | stinkbug (Halyomorpha halys) |
| Maca- | integrifolla | Maca- | Anthracnose (Colletotrichum |
| damia | | damia | gloeosporioides), |
| | | | raceme blight (Botrytis cinerea), |
| | | | trunk & stem canker |
| | | | (Phytophthora cinnamomi), |
| | | | macadamis nut borer |
| | | | (Cryptophlebia ambrodelta) |
| Malus | domestica | Apple | Apple scab (Venturia inaequalis), |
| | | | cedar apple rust |
| | | | (Gymnosporangium |
| | | | juniperi-virginianae), flyspeck |
| | | | (Zygophiala jamaicensis), |
| | | | powdery mildew (Podosphaera |
| | | | leucotricha), fire blight (Erwinia |
| | | | amylovora), aphids (Aphis |
| | | | pomi, Eriosoma lanigerum), |
| | | | apple maggot |
| | | | (Rhagoletis pomonella), |
| | | | codling moth (Cydia pomonella), |
| | | | leafhoppers (Typhlocyba pomaria), |
| | | | leafrollers (Platynota stultana), |
| | | | brown marmorated stinkbug |
| | | | (Halyomorpha halys), |
| | | | spider mites (Tetranychus urticae) |
| Medicago | sativa | Alfalfa | Common leaf spot |
| | | | (Pseudopeziza medicaginis), |
| | | | alfalfa caterpillar (Colias |
| | | | eurytheme), alfalfa weevil |
| | | | (Hypera postica), aphids |
| | | | (Aphis craccivora, |
| | | | Acyrthosiphon pisum), |
| | | | beet armyworm (Spodoptera |
| | | | exigua) |
| Musa | paradisiaca | Banana | Anthracnose (Colletotrichum |
| | | | musae), black sigatoka |
| | | | (Mycosphaerella fijiensis), |
| | | | cigar end rot (Verticillium |
| | | | fructigena, Trachysphaera |
| | | | theobromae), Panama disese |
| | | | (Fusarium oxysporum), rhizome |
| | | | rot (Erwinia caratovora, E. |
| | | | chrysanthemi), banana aphid |
| | | | (Pentalonia nigronervosa), |
| | | | banana weevil |
| | | | (Cosmopolites sordidus) |
| Myristica | fragrans | Nutmeg | Leaf spot (Colletotrichum |
| | | | gloeosporioides), cocoa |
| | | | weevil (Aracerus fasciculatus) |
| Olea | europaea | Olive | Armillaria root rot |
| | | | (Armillaria mellea), olive knot |
| | | | (Pseudomonas savastanoi), |
| | | | root rot (Phytophthora |
| | | | citricola, P. dreschleri), |
| | | | verticillium wilt (Verticillium |
| | | | dahliae), olive fruit fly |
| | | | (Bactrocera oleae), olive mite |
| | | | (Oxzycenus maxwelli), |
| | | | olive psyllid (Euphyllura |
| | | | olivina), thrips (Frankliniella |
| | | | occidentalis) |
| Oryza | sativa | Rice | Bacterial blight (Xanthomonas |
| | | | oryzae), brown spot |
| | | | (Cochiobolus miyabeanus), |
| | | | narrow leaf spot (Cercospora |
| | | | oryzae), rice blast (Magnaporthe |
| | | | grisea), sheath blight |
| | | | (Rhizoctonia solani), leafhoppers |
| | | | (Nephotettix spp., Recilia dorsalis, |
| | | | Nilaparvata lugens, Laodelphax |
| | | | striatellus, Sogatell furciferra), |
| | | | rice bug (Leptocorisa oratorius, |
| | | | L. acuta), rice mealybugs |
| | | | (Brevennia rehi), stem borers |
| | | | (Scirpophaga incertulas, |
| | | | Chilo suppressalis, S. |
| | | | innotata) |
| Phaseolus | vulgaris | Bean | Alternaria leaf spot (Alternaria |
| | | | alternata), bean rust (Uromyces |
| | | | appendiculatus), fusarium root |
| | | | rot (Fusarium solani), white |
| | | | mold (Sclerotinia sclerotium), |
| | | | bacterial blight (Xanthomonas |
| | | | campestris), halo blight |
| | | | (Pseudomonas savastanoi), |
| | | | aphids (Aphis craccivora, |
| | | | Acyrthosiphon pisum), |
| | | | armyworms (Spodoptera |
| | | | exigua, S. praefica), corn |
| | | | earworm (Helicoverpa zea), |
| | | | leafminers (Liriomyza spp.), |
| | | | loopers (Trichoplusia ni, |
| | | | Autographa californica), |
| | | | Mexican bean beetle (Epilachna |
| | | | varivestis), spider mites |
| | | | (Tetranychus urticae) |
| | lunatus | Lima | Alternaria leaf spot (Alternaria |
| | | bean | alternata), bean rust (Uromyces |
| | | | appendiculatus), fusarium root rot |
| | | | (Fusarium solani), white mold |
| | | | (Sclerotinia sclerotium), bacterial |
| | | | blight (Xanthomonas campestris), |
| | | | halo blight (Pseudomonas |
| | | | savastanoi), |
| | | | aphids (Aphis craccivora, |
| | | | Acyrthosiphon pisum), |
| | | | armyworms (Spodoptera exigua, |
| | | | S. praefica), corn earworm |
| | | | (Helicoverpa zea), leafminers |
| | | | (Liriomyza spp.), loopers |
| | | | (Trichoplusia ni, Autographa |
| | | | californica), Mexican bean beetle |
| | | | (Epilachna varivestis), spider |
| | | | mites (Tetranychus urticae) |
| Pinus | taeda | Loblolly | Fusiform rust (Cronartium |
| | | pine | quercuum), weevils (Hylobius |
| | | | pales, Pachylobius picivorus), |
| | | | pine tip moth (Rhyacionia |
| | | | frustrana), pine webworm |
| | | | (Tetralopha robustella), pine |
| | | | sawflies (Neodiprion excitans, |
| | | | N. lecontei, N. taedae), |
| | | | southern pine coneworm |
| | | | (Dioryctria amatella) |
| | ponderosa | Ponderosa | Annosus root rot (Hetero- |
| | | pine | basidion irregulare), root rots |
| | | | (Armillaria ostoyae), black |
| | | | stain root disease (Grasmannia |
| | | | wageneri), brown cubical sap rot |
| | | | (Gloeophyllum sepiarium), |
| | | | conifer rust (Malampsora |
| | | | occidentalis), pine needle cast |
| | | | (Lophodermella concolor), red |
| | | | heart rot (Stereum |
| | | | sanguinolentum) tussock |
| | | | moth (Orgyia psedotsugata), |
| | | | European pine shoot moth |
| | | | (Rhyacionia buoliana), |
| | | | mountain pine beetle |
| | | | (Dendroctonus ponderosae) |
| Pistacia | vera | Pistachio | Alternaria late blight |
| | | | (Alternaria alternata), Armillaria |
| | | | root rot (Armillaria mellea), |
| | | | panicle & shoot blight |
| | | | (Botryosphaeria dothidea), |
| | | | powdery mildew |
| | | | (Phyllactinia guttata), rust |
| | | | (Uromyces terebinthi), septoria |
| | | | leaf spot (Septoria spp.), |
| | | | pistachio dieback (Xanthomonas |
| | | | translucens), pistachio psyllid |
| | | | (Agonoscena targionii), |
| | | | pistachio twig borer |
| | | | (Kermania pistaciella) |
| Pisum | sativum | Pea | Common root rot |
| | | | (Aphanomyces euteiches), |
| | | | ascochyta disease (Mycosphaerella |
| | | | pinodes, Phoma medicaginis, |
| | | | Ascochyta pisi), downy |
| | | | mildew (Peronospora viciae), |
| | | | fusarium root rot (Fusarium |
| | | | solani), gray mold (Botrytis |
| | | | cinerea), powdery mildew |
| | | | (Erysiphe pisi), septoria blotch |
| | | | (Septoria pisi), bacterial |
| | | | blight (Pseudomonas syringae), |
| | | | aphids (Acyrthosiphon pisum, |
| | | | Myzus persicae), leafminers |
| | | | (Liriomyza spp.), Mexican bean |
| | | | beetle (Epilachna varivestis), |
| | | | thrips (Frankliniella occidentalis, |
| | | | Thrips tabaci), root knot |
| | | | nematode (Meloidogyne spp.), |
| | | | spider mites (Tetranychus urticae) |
| Prunus | dulcis | Almond | Alternaria leaf spot (Alternaria |
| | | | alternata), anthracnose |
| | | | (Colletotrichum acutatum), brown |
| | | | rot (Monolinia laxa), shot |
| | | | hole (Wilsonmyces carpophilus), |
| | | | almond brownline (peach yellow |
| | | | leafroll mycoplasma), |
| | | | almond leaf scorch (Xylella |
| | | | fastidiosa), navel orangeworm |
| | | | (Amyelois tranitella), peach |
| | | | twig borer (Anarsia lineatella), |
| | | | peachtree borer (Synanthedon |
| | | | exitlosa), brown marmorated |
| | | | stinkbug (Halyomorpha halys) |
| | armeniaca | Apricot | Armillaria root rot |
| | | | (Armillaria mellea), |
| | | | brown rot (Monlinia |
| | | | laxa), Eutypa dieback |
| | | | (Eutypa lata), jacket |
| | | | rot (Botrytis cinerea, |
| | | | Sclerotinia sclerotiorum), |
| | | | powdery mildew (Sphaerotheca |
| | | | pannosa, Podosphaera |
| | | | tridactyla), shot hole |
| | | | (Wilsonmyces carpophilus), |
| | | | bacterial canker (Pseudomonas |
| | | | syringae), root & crown rot |
| | | | (Phytophthora spp.), plum |
| | | | pox virus (PPV), fruittree |
| | | | leafroller (Archips |
| | | | argyrospila), peach twig borer |
| | | | (Anarsia lineatella), peachtree |
| | | | borer (Synanthedon exitlosa), |
| | | | brown marmorated stinkbug |
| | | | (Halyomorpha halys) |
| | avium | Cherry | Armillaria root rot (Armillaria |
| | | | mellea), black knot (Apiosporina |
| | | | marbosa), brown rot (Monilinia |
| | | | fructicola, M. laxa), cherry leaf |
| | | | spot (Cocomyces hiemalis), |
| | | | powdery mildew |
| | | | (Podosphaera spp.), |
| | | | rust (Tranzschella discolor), |
| | | | silver leaf (Chondrosterum |
| | | | pupureum), crown & |
| | | | root rot (Phytophthora spp.), X- |
| | | | disease (X-disease mycoplasma), |
| | | | cherry canker (Pseudomonas |
| | | | syringae), aphids (Myzus cerasi), |
| | | | peach twig borer (Anarsia |
| | | | lineatella), western cherry |
| | | | fruit fly (Rhagoletis indifferens), |
| | | | brown marmorated stinkbug |
| | | | (Halyomorpha halys), spotted wing |
| | | | drosophila (Drosophila |
| | | | suzukii), spider mites |
| | | | (Tetranychus urticae) |
| | persica | Peach, | Scab (Caldosporium carpophilum), |
| | | nectarine | brown rot (Monilinia fructicola), |
| | | | rust (Tranzschella discolor), |
| | | | shot hole (Wilsonomyces |
| | | | carpophilus), leaf curl |
| | | | (Taphrina deformans), bacterial |
| | | | canker (Pseudomonas syringae), |
| | | | bacterial spot (Xanthomonas |
| | | | campestris), plum poxvirus |
| | | | (PPV), fruittree leafroller |
| | | | (Archips argyrospila), oriental |
| | | | fruit moth (Grapholitha molesta), |
| | | | peach twig borer (Anarsia |
| | | | lineatella), brown marmorated |
| | | | stinkbug (Halyomorpha halys), |
| | | | spotted wing drosophila |
| | | | (Drosophila suzukii), spider |
| | | | mites (Tetranychus urticae) |
| | domestica | Plum, | Armillaria root rot (Armillaria |
| | | prune | mellea), black knot (Apiosporina |
| | | | marbosa), brown rot |
| | | | (Monilinia fructicola, M. laxa), |
| | | | powdery mildew (Podosphaera |
| | | | pannosa, P. tridactyla), rust |
| | | | (Tranzschella discolor), bacterial |
| | | | canker (Pseudomonas syringae), |
| | | | bacterial spot (Xanthomonas |
| | | | campestris), plum pox virus |
| | | | (PPV), fruittree leafroller |
| | | | (Archips argyrospila), oriental |
| | | | fruit moth (Grapholitha molesta), |
| | | | peach twig borer (Anarsia |
| | | | lineatella), brown marmorated |
| | | | stinkbug (Halyomorpha |
| | | | halys), spotted wing |
| | | | drosophila (Drosophila suzukii) |
| Pseudo- | menziesii | Douglas | Armillaria root rot (Armillaria |
| tsuga | | Fir | mellea), black stain |
| | | | root disease (Ceratocystis |
| | | | wageneri), laminated root rot |
| | | | (Phellinus weirii), canker |
| | | | (Diaporthe lokoyae, Phomo |
| | | | spp.), twig weevils |
| | | | (Cylindrocopturnus furnissi), |
| | | | engraver beetles (Scolytus |
| | | | unispinosus), flatheaded wood |
| | | | borers (Melanophila drummondi), |
| | | | Douglas-fir beetles |
| | | | (Dendroctonus pseudotsugae) |
| Punica | granatum | Pome- | Cercospora fruit spot (Cercospora |
| | | granate | punicae), heart |
| | | | rot (Alternaria spp.), |
| | | | aphids (Aphis gossypii), |
| | | | pomegranate fruit |
| | | | borer (Virahola isocrates) |
| Pyrus | communis | Pear | Armillaria root rot (Armillaria |
| | | | mellea), crown & |
| | | | root rot (Phytophthora |
| | | | spp.), blast (Pseudomonas |
| | | | syringae), fire blight (Erwinia |
| | | | amylovora), brown marmorated |
| | | | stinkbug (Halyomorpha halys), |
| | | | codling moth (Cydia |
| | | | pomonella), leafrollers (Platynota |
| | | | stultana, Agyrotaenia velutinana), |
| | | | pear psylla (Psylla pyricola), |
| Rubus | ursinus | Black- | Anthracnose (Elsinoe veneta), |
| | | berry | fruit rot (Botrytis |
| | | | cinerea), orange rust |
| | | | (Gymnoconia peckiana), |
| | | | powdery mildew (Podosphaera |
| | | | macularis), Japanese |
| | | | beetle (Popillia japonica), |
| | | | leafrollers (Platynota stultana, |
| | | | Pandemis pyrusana, Epiphyas |
| | | | postvittana, Argyrotaenia |
| | | | franciscana), rednecked cane |
| | | | borer (Agrilus ruficollis), |
| | | | spotted wing Drosophila |
| | | | (Drosophila suzukii) |
| | idaeus | Red | Cane blight (Leptosphaeria |
| | | raspberry | coniothyrium), gray mold |
| | occidentalis | Black | (Botrytis cinerea), fire |
| | | raspberry | blight (Erwinia amylovora), |
| | | | Phytophthora root rot |
| | | | (Phytophthora fragariae), |
| | | | weevils (Otiorhynchus spp.), |
| | | | two-spotted spider mite |
| | | | (Tetranychus urticae), |
| | | | spotted wing Drosophila |
| | | | (Drosophila suzukii), |
| Secale | cereale | Rye | Powdery mildew (Erysiphe |
| | | | graminis), rust |
| | | | (Puccinia reconita), aphids |
| | | | (Rhopalosuphum padi, Diuraphis |
| | | | noxia, Sitobioni avenae) |
| Solanum | tuberosum | Potato | Common scab (Streptomyces |
| | | | spp.), black scurf |
| | | | (Rhizoctonia solani), |
| | | | gray mold (Botrytis cinerea), |
| | | | early blight (Alternaria solani), |
| | | | late blight (Phytophthora |
| | | | infestans), zebra chip (Candidatus |
| | | | Liberibacter solanacearum), |
| | | | aphids (Myzus persicae, |
| | | | Macrosiphon euphorbiae), Colorado |
| | | | potato beetle (Leptinotarsa |
| | | | decemlineata), potato psyllid |
| | | | (Bactericera cockerelli), pale cyst |
| | | | nematode (Globodera pallida) |
| Sorghum | bicolor | Sorghum | Head smut (Sphacelotheca |
| | | | reliana), downy |
| | | | mildew (Peronosclerospora |
| | | | sorghi), stalk rot |
| | | | (Fusarium moniliforme, |
| | | | Macrophomina phaseolina), |
| | | | ergot (Claviceps africana), |
| | | | anthracnose (Colletotrichum |
| | | | graminicola), sorghum midge |
| | | | (Contarinia sorghicola), lesser |
| | | | cornstalk borer, fall armyworm |
| | | | (Spodoptera frugiperda), corn |
| | | | earworm (Helicoverpa zea), |
| | | | sorghum webworm (Celama |
| | | | sorghiella), chinch bugs |
| | | | (Blissus leucopterus) |
| Spinacia | oleracea | Spinach | Anthracnose (Colletotrichum |
| | | | spp.), downy |
| | | | mildew (Peronospora farinosa), |
| | | | Fusarium wilt (Fusarium |
| | | | oxysporum), white rust |
| | | | (Albugo occidentalis), aphids |
| | | | (Myzus persicae, Macrosiphon |
| | | | euphorbiae), armyworms |
| | | | (Spodoptera |
| | | | exigua, S. praefica), cabbage |
| | | | looper (Trichoplusia ni), crown |
| | | | mite (Rhizoglyphus spp.) |
| Triticum | aestivum | Wheat | Ergot (Claviceps purpurea), |
| | | | eyespot (Oculimacula spp.), |
| | | | Fusarium head blight (Fusarium |
| | | | spp.), powdery mildew |
| | | | (Erysiphe graminis), rusts (Puccinia |
| | | | graminis, P. triticina, P. |
| | | | striiformis), aphids (Rhopalosuphum |
| | | | padi, Diuraphis noxia, Sitobion |
| | | | avenae), armyworms (Mythimna |
| | | | unipunctata, Spodoptera praefica) |
| Vitis | vinifera | Grapes | Anthracnose (Elsinoe ampelina), |
| | | | Armillaria root rot |
| | | | (Armillaria mellea), bunch rot |
| | | | (Botrytis cinerea), downy |
| | | | mildew (Plasmopara viticola), black |
| | | | rot (Guignardia bidwellii), |
| | | | dieback (Eutypa lata), esca |
| | | | (Phaemoniella aleophilum, |
| | | | P. Chlamydospora), powdery |
| | | | mildew (Erysiphe necator), |
| | | | Pierce's disease (Xylella |
| | | | fastidiosa), black vine weevil |
| | | | (Otiorhynchus sulcatus), grape |
| | | | cane girdler (Ampeloglypter |
| | | | ater), omnivorous leafroller |
| | | | (Platynota stultana), grape |
| | | | mealybug (Pseudococcus |
| | | | maritimus), grape phylloxera |
| | | | (Daktulosphaira vitifollae), |
| | | | glassy winged sharpshooter |
| | | | (Homalodisca vitripennis), thrips |
| | | | (Franklinlella occidentalis) |
| Zea | mays | Corn | Anthracnose (Colletotrichum |
| | | | graminicola), rust |
| | | | (Puccinia sorghi), smut |
| | | | (Ustilago zeae), northern leaf |
| | | | blight (Exserohilium turcicum), |
| | | | southern corn leaf blight |
| | | | (Bipolaris maydis), Goss's blight |
| | | | (Clavibacter michiganensis), |
| | | | aphids (Rhopalosiphum |
| | | | maidis), corn earworm |
| | | | (Helicoverpa zea), fall armyworm |
| | | | (Spodoptera frugiperda), flea beetles |
| | | | (Chaetocnema pulicaria), root knot |
| | | | nematode (Meloidogyne incognita) |

TABLE 5
Amino acid sequences of representative targeting peptides.

| Source | Sequence | Target |
|---|---|---|
| Rubisco small | MASSVLSSAAVATRSNVAQANMVAPFTGLKSAASFPV | chloroplast |
| subunit (tobacco) | SRKQNLDITSIASNGGRVQC (SEQ ID NO: 52) | |
| Arabidopsis | MRILPKSGGGALCLLFVFALCSVAHS (SEQ ID | cell wall/ |
| proline-rich | NO: 53) | secretory |
| protein 2 | | pathway |
| (AT2G21140) | | |
| PTS-2 (conserved | RLX₅HL (SEQ ID NO: 54) | peroxisome |
| in eukaryotes) | MRLSIHAEHL (SEQ ID NO: 55) | |
| | SKL | |
| Arabidopsis | MLRTVSCLASRSSSSLFFRFFRQFPRSYMSLTSSTAA | mitochondria |
| presequence | LRVPSRNLRRISSPSVAGRRLLLRRGLRIPSAAVRSV | and |
| protease1 | NGQFSRLSVRA (SEQ ID NO: 56) | chloroplast |
| (AT3G19170) | | |
| Chlamydomonas | MALVARPVLSARVAASRPRVAARKAVRVSAKYGEN | chloroplast |
| reinhardtii- | (SEQ ID NO: 57) | |
| (Stroma- | MQALSSRVNIAAKPQRAQRLVVRAEEVKA | |
| targeting | (SEQ ID NO: 58) | |
| cTPs: | MQTLASRPSLRASARVAPRRAPRVAVVTKAALDPQ | |
| photosystem | (SEQ ID NO: 59) | |
| I (PSI) | MQALATRPSAIRPTKAARRSSVVVRADGFIG | |
| subunits P28, | (SEQ ID NO: 60) | |
| P30, P35 and | | |
| P37, | | |
| respectively) | | |
| C. reinhardtii- | MAFALASRKALQVTCKATGKKTAAKAAAPKSSGVEFY | chloroplast |
| chlorophyll a/b | GPNRAKWLGPYSEN (SEQ ID NO: 61) | |
| protein (cabll-1) | | |
| C. reinhardtii- | MAAVIAKSSVSAAVARPARSSVRPMAALKPAVKAAPV | chloroplast |
| Rubisco small | AAPAQANQMMVWT (SEQ ID NO: 62) | |
| subunit | | |
| C. reinhardtii- | MAAMLASKQGAFMGRSSFAPAPKGVASRGSLQVVAGL | chloroplast |
| ATPase-γ | KEV (SEQ ID NO: 63) | |
| Arabidopsis | CVVQ (SEQ ID NO: 64) | membrane |
| thaliana | | |
| abscisic acid | | |
| receptor PYL10 | | |

X₅ means any five amino acids can be present in the sequence to target the protein to the peroxisome (e.g. RLAVAVAHL, SEQ ID NO: 65).

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1. Inoculation/Generation of a Symbiont Forming Inoculum and Symbiont

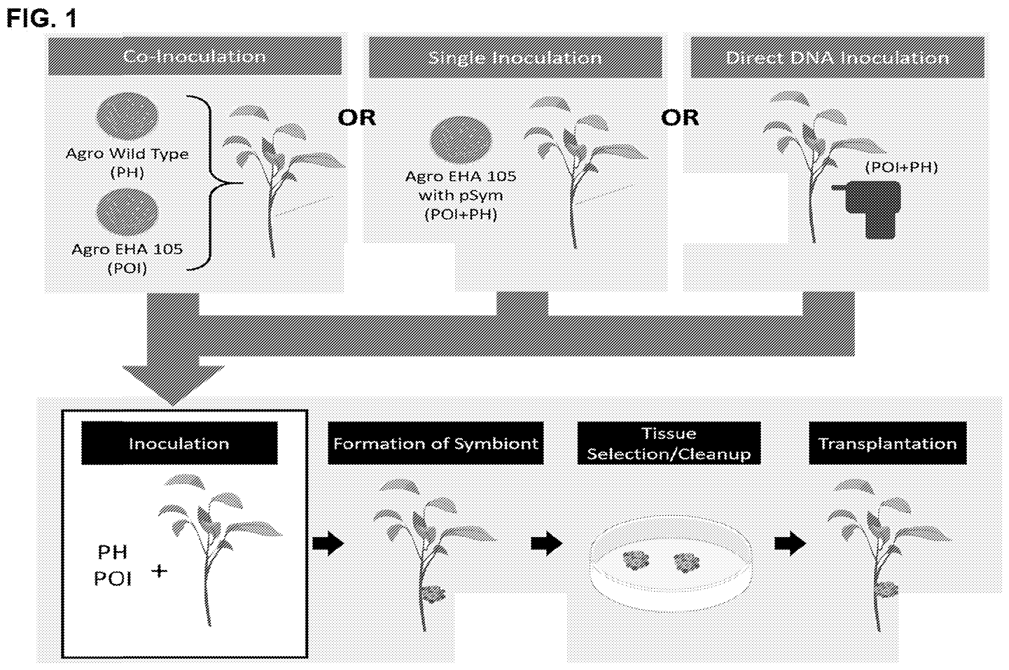

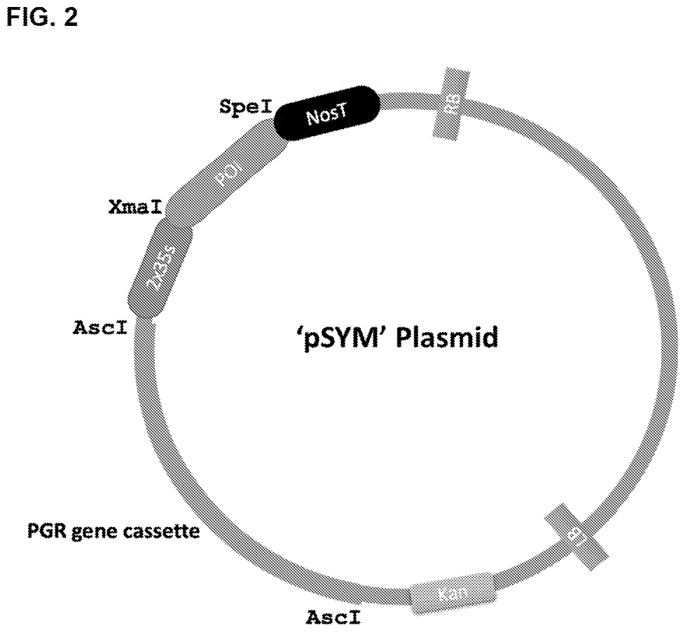

A symbiont forming inoculum and symbiont can be generated using several different methods, which include: i) co-inoculation, ii) single inoculation, and iii) direct DNA inoculation as illustrated in FIG. 1.
i. Co-inoculation method employs two Agrobacterium spp. strains. One strain is a disarmed Agrobacterium spp. that contains a binary vector (e.g., A. tumefaciens strain EHA105 strain) which is used to express a polynucleotide of interest (POI) and a second wild type (WT) Agrobacterium strain that is used to transfer phytohormone genes (PHG) to the plant cells. Plant cells that are co-inoculated in this manner, having both the POI and PH genes (PHG) can be referred to as symbiont forming inoculum or as a symbiont depending on the intended use. In some cases, the cells can be used as symbiont forming inoculum to form a symbiont on a host plant or when cells located on a plant (or part thereof) are inoculated in this manner with the bacterial cells, they can form a symbiont directly on the plant.
   The disarmed Agrobacterium strain carrying a binary vector, in this example, strain A. tumefaciens EHA105, and the WT strain were grown using procedures common in the art and then each strain was centrifuged to recover a bacterial pellet and then resuspended in inoculation buffer (10 mM MgCl2, 10 mM MES [pH 5.6], 100 μM acetosyringone) to a final concentration of 1 and 0.1 at OD₆₀₀ respectively. These were then kept at room temperature for 1-3 hours and then mixed together before the inoculation of plant tissue following which the symbiont forming inoculum or the symbiont were formed.
ii. For a single inoculation method, only a single Agrobacterium spp. is used to inoculate a plant cell or a plant (e.g., a host plant). In this example, the disarmed Agrobacterium tumefaciens EHA105 strain carrying a binary vector (e.g., pSYM plasmid, see FIG. 2) comprising both POI and PHG was used to inoculate plant cells. The pSYM plasmid contains a cassette of approximately 7.5 Kb plant growth regulators, (indole-3-acetamide hydrolase, tryptophan 2-monooxygenase, isopentenyl transferase, indole-3-lactate synthase) and a POI operably linked to a constitutive or inducible promoter. The pSYM plasmid also contained a selectable marker gene (kanamycin) to allow selection of Agrobacterium spp. cells carrying the pSYM plasmid. Plant tissue is inoculated with a suspension of the pSYM containing Agrobacterium spp. to form the symbiont forming inoculum or the symbiont.
iii. For direct DNA inoculation, biolistic delivery systems can be used to deliver DNA into a plant cell or tissue. This is done using POI and PHG genes coated metal particles that are propelled directly into the host plant cells without the use of Agrobacterium spp. as the gene(s) vector. The cell then incorporates the POI and PHG genes into the genome and then the plant tissue can form into either a symbiont forming inoculum or a symbiont. Many other methods of direct DNA delivery are known and can be used instead of biolistics including, for example, electroporation, microinjection, lipofection (liposome mediated transformation), sonication, silicon fiber mediated transformation, chemically stimulated DNA uptake (e.g., polyfection; e.g., polyethylene glycol (PEG) mediated transformation), and/or laser microbeam (UV) induced transformation with similar success.

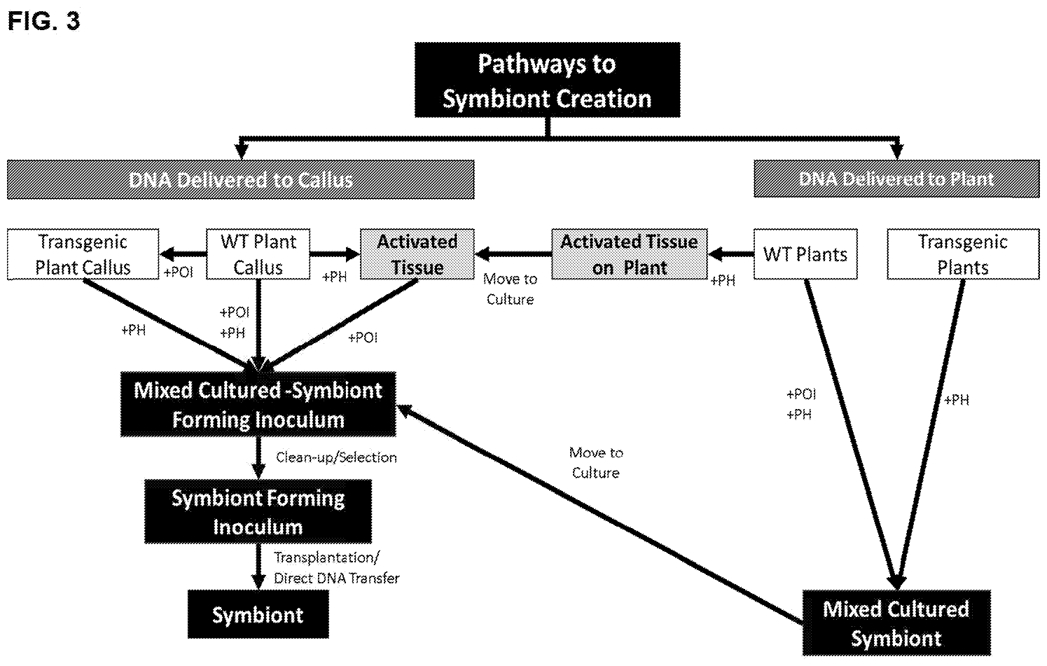

Once the DNA is delivered into the host plant cell genome, the expression of the PHG will induce and stimulate plant tissues to grow a mixed cultured symbiont (see FIG. 3) having a collection of cells with different gene insertions and expression levels of POI and PHG. The mixed cultured symbiont can grow autonomously and connect to the host plant via vascularization by connecting with one or both the phloem and xylem where the POI product may be transported to the host plant. The POI product produced by symbiont may be transferred via the apoplast and/or the symplast from the symbiont and/or through the phloem and/or xylem for dispersion throughout the host plant.

The mixed cultured symbiont can subsequently be excised and grown in hormone free culture where cells can be selected having desirable traits and expression level, in doing so allowing for isolation of uniform symbiont forming inoculum(s). Selection for pure culture symbiont forming inoculum(s) can include, but is not limited to, the use of antibiotic selection (e.g. using an antibiotic resistance marker POI that will allow the growth of only transformed cells (i.e., cells with the POI and PHG)), serial dilution/division of the culture, or may be converted to a protoplast and single protoplast cells that can be isolated and grown up to a pure culture. Symbiont forming inoculum(s) can be selected for those expressing desirable attributes in addition to the expression of the POI and PHG.

In addition, the process of using antibiotics can also be used to eliminate Agrobacterium cells from the mixed cultured symbiont forming inoculum when Agrobacterium is used in the symbiont forming process.

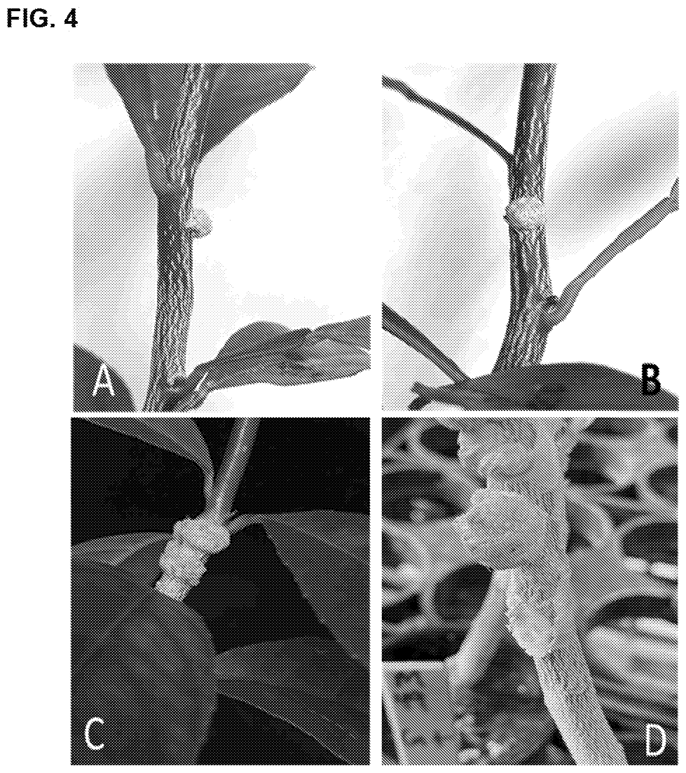

The final procedure involves the transplantation of the selected symbiont forming inoculum(s) onto a host plant where it can attach and provide the POI expression product or a product of the POI expression product (e.g., the POI expression product can be an enzyme that is involved in the biosynthesis of a product in the symbiont, and it is the product that is transported out of the symbiont and into the host plant) for dispersion into and or throughout the plant. Once the symbiont forming inoculum(s) is/are attached to the plant host it forms what is termed a symbiont(s). An example of a symbiont is shown in FIG. 4 where panels A and B show citrus symbionts formed after 60 days post-inoculation using a co-inoculation method. Panels C and D show symbionts formed using a single strain-inoculation method on citrus (see, e.g., FIG. 1 for graphical representation of co-inoculation and single strain inoculation).

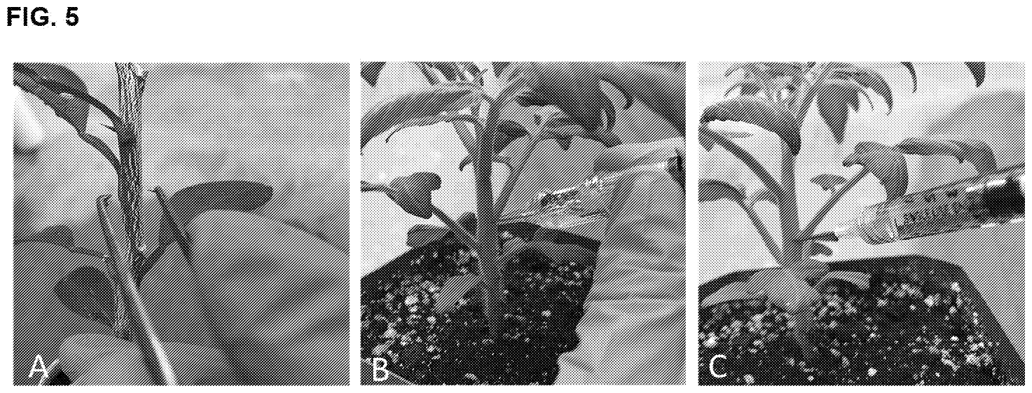

In this example, Agrobacterium spp. carrying the pSYM was used to inoculate a plant host and induced symbiont formation. To do this, Agrobacterium spp. was grown in 10 mL Luria Bertani broth supplemented with appropriate antibiotics (50 μg of kanamycin) at 28° C. overnight. Both strains were centrifuged to recover a pellet of bacterial cells and then this was resuspended in inoculation buffer (previously described). Different techniques may be used to inoculate host plants. For instance, woody plants like citrus that have a tough external structure on the stem require a method to pierce the woody stem tissue to penetrate into the plant. Here toothed tweezers for citrus (see FIG. 5, panel A) dipped in Agrobacterium spp. inoculation solution may be used to pierce the citrus bark tissue to deliver the solution to the plant. Herbaceous plants like tomatoes (FIG. 5, panel B and FIG. 5, panel C) that have a flexible stem were inoculated in this example using a tattoo needle (FIG. 5, panel B) or a syringe needle (FIG. 5, panel C) to inject or pass the Agrobacterium spp. solution into the plant tissue by simply dipping a needle in the Agrobacterium spp. solution and piercing the tissue.

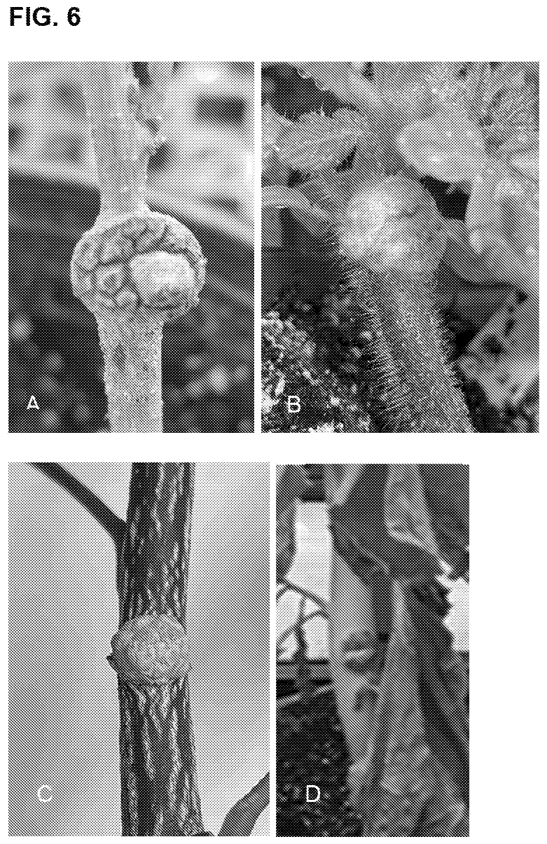

Symbiont tissue can be grown on a range of different host plant types. In FIG. 6, we illustrate symbiont formation and growth on pecan (FIG. 6, panel A), tomato (FIG. 6, panel B), citrus (FIG. 6, panel C), and Nicotiana benthamiana (FIG. 6, panel D). These symbionts were formed by inoculation using one of the methods described above.

### Example 2. Symbiont-Forming Inoculum In Vitro Culture

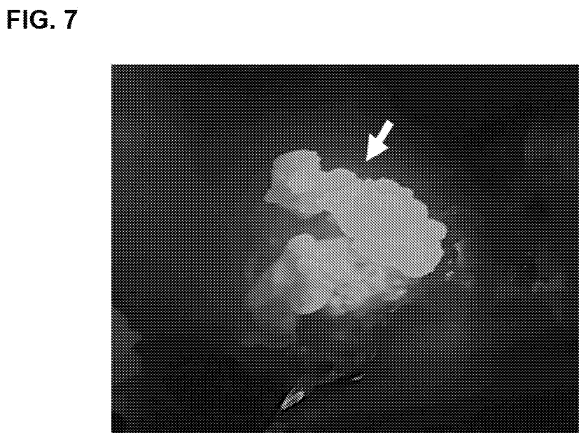

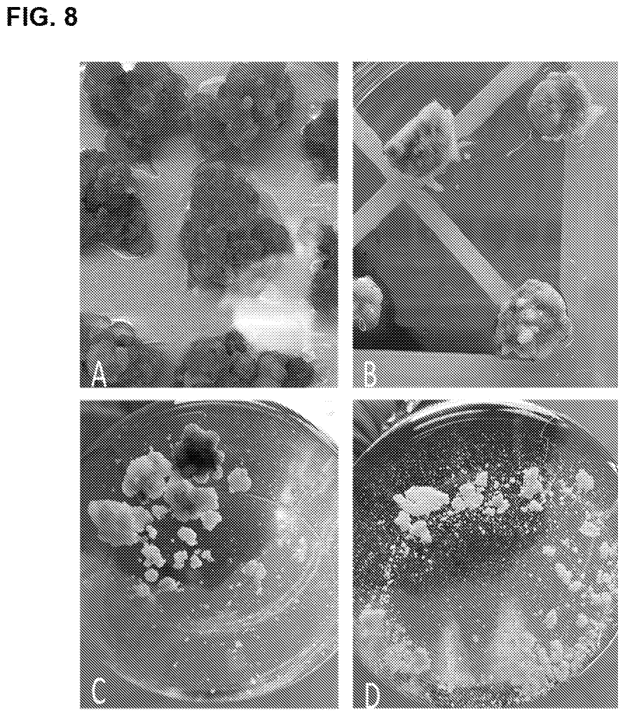

A symbiont forming inoculum (e.g. FIG. 7) can be generated and used to inoculate additional host plants. This example describes a process for cleaning the symbiont tissue of microbial contamination including the removal of Agrobacterium spp., or other bacteria used to generate a symbiont, and any microbial impurities that might contaminate the agar or liquid cultures (e.g. FIG. 8). This process allows for the generation and maintenance of symbiont forming inoculum in vitro culture.

Once the symbiont develops on a host plant, it can be used to generate symbiont forming inoculum (FIG. 7 and FIG. 8). For this purpose, symbiont tissue is removed from the host plant and rinsed with running tap water for about 30 minutes. The rinsed tissue is then washed with ethanol. Subsequently, tissue was washed with a 10% bleach solution followed by a wash with a solution of sterile water. The sterilization steps are done using aseptic technique in sterile conditions in a laminar flow hood to avoid external contamination of bacteria or fungi.

After the sterilization steps, the tissues were placed onto sterile paper to dry (e.g., sterile filter paper) and then placed onto Murashige and Skoog (MS) based solid agar media for both tomato and citrus (FIG. 8, panels A and B) or in liquid agar media for both tomato and citrus (FIG. 8, panels C and D).

A growth media comprising an antibiotic was used in the cell culture to remove Agrobacterium spp. cells and provide only symbiont forming inoculum cells. After multiple tissue culture divisions and passages on media, a homogeneous expression of the POI is provided as shown in FIG. 7. FIG. 7 shows symbiont forming inoculum expressing mCherry on selective media with high expression of the fluorescent marker as shown under UV light and mCherry filter.

### Example 3. Transplantation of Symbiont Forming Inoculum onto Host Plants

Symbiont tissues (FIG. 6) were isolated from different crops and grown on selective agar media to remove bacteria as described in Example 2, producing symbiont forming inoculums on culture media. Symbiont forming inoculum tissue transformed with mCherry (FIG. 7) or green fluorescent protein (GFP) was used to optimize tissue selection by screening fluorescent intensity using mCherry/GFP filters with UV lamp and by transferring only the fluorescent cells to new selecting agar media multiple times (as described in Example 2). Symbiont forming inoculum tissues from tomato and citrus were grown under selective solid agar media conditions (FIG. 8, panel A (tomato); FIG. 8, panel B (citrus)) and under selective liquid agar media conditions (FIG. 8, panel C (tomato) and (FIG. 8, panel D (citrus)).

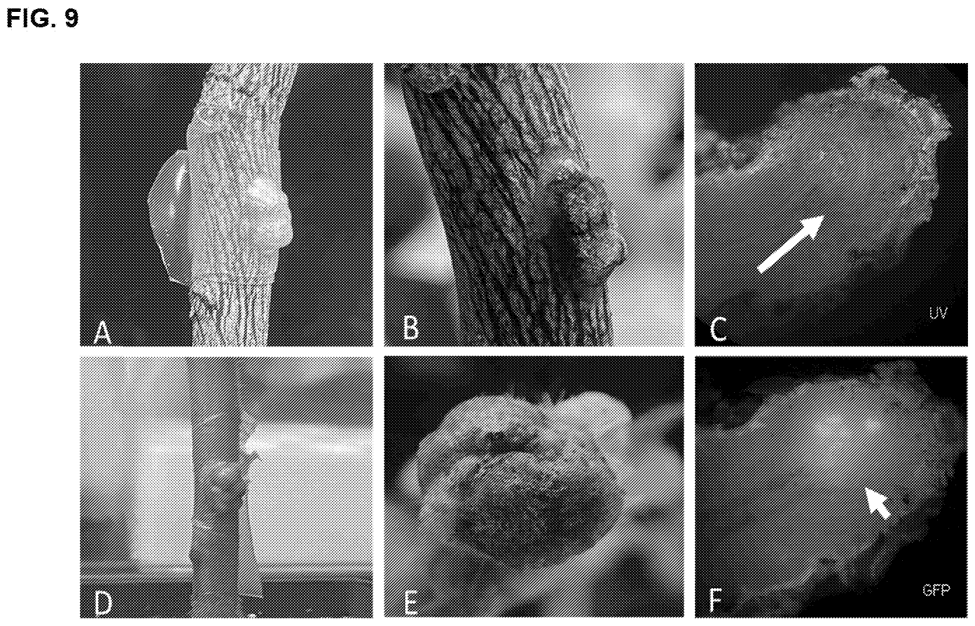

Symbiont forming inoculum tissue that was ready for transplantation was removed from the culture media and then washed in a transplantation solution containing phytohormones (sterile distilled water with auxin and cytokinin). The transplantation solution is used to aid in the transplantation efficacy of the symbiont forming inoculum and host plant interaction. After washing, symbiont forming inoculum in the transplantation solution, tissue from citrus was applied to a citrus plant stem at a location where the stem epidermal layers had been previously removed. To ensure transplantation/graft adhesion of the symbiont forming inoculum to form a symbiont, silicon tape was firmly applied around the symbiont forming inoculum/symbiont tissue and the stem (FIG. 9, panel A). As would be well understood, other methods for keeping the symbiont forming inoculum/symbiont tissue in place on the host plant made be used instead of silicon tape. After about six weeks the silicon tape was removed from the symbiont (FIG. 9, panel B) and tissue was excised to evaluate adhesion, vascularization (FIG. 9, panel C) and GFP expression (FIG. 9, panel F), each of which was observed.

For tomato, symbiont forming inoculum tissue prepared from tomato was first washed in a transplantation solution containing phytohormones (auxin and cytokinin). The symbiont forming inoculum tissue from tomato was applied to a tomato plant stem with the stem epidermal layer removed. Similar to the citrus example, in order to ensure adhesion of the symbiont tissue to the stem, a plastic wrap (e.g. Parafilm® M) was applied to assist in maintaining humidity and contact between the symbiont forming inoculum and the stem (FIG. 9, panel D). After six weeks the symbiont tissue had integrated with the tomato host plant and increased in size (FIG. 9, panel E) demonstrating successful transplantation.

### Example 4. Symbiont Versatility

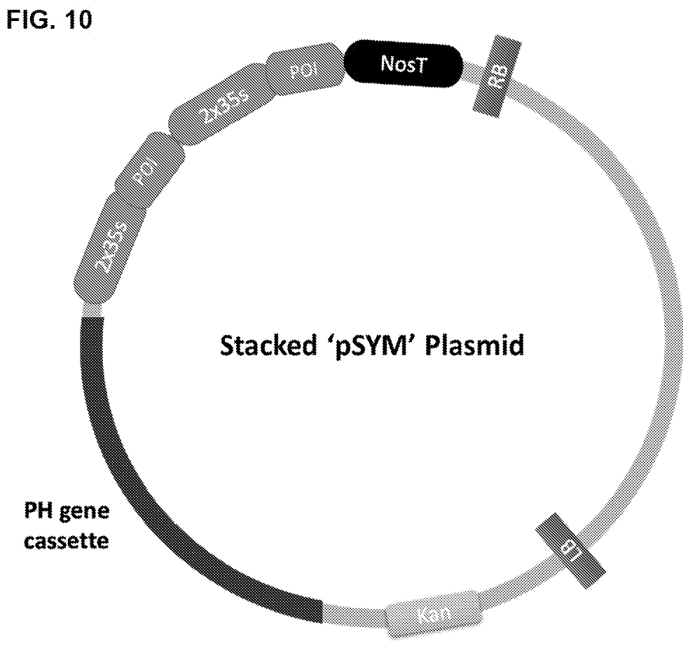

Symbiont cells can express one or two or more POIs introduced using one or two or more vectors/expression cassettes that can be provided to the cells in one or two or more steps (e.g., one or two or more inoculations (e.g., one or more than one agrobacterium strain); one or more than one introduction using any system known to deliver DNA. Such methods are exemplified in FIG. 1. In addition to transplanting different symbiont types onto a host plant (i.e. one symbiont with POI of one type and one or more additional symbionts comprising one or more different POIs) onto a host plant, it is also possible to generate a pSYM plasmid having multiple polynucleotides of interest on the same vector/expression cassette/T-DNA region—effectively ‘stacking’ multiple POIs on a single pSYM to be delivered to form a symbiont (as previously described in Examples 1-3). Such POIs can each be regulated by a specific promoter (FIG. 10) or may be regulated by separate promoters, which may be the same promoter or different promoters. It is also possible to use different Agrobacterium spp. (or other viable bacterial systems) each carrying a unique pSYM having only one POI each (FIG. 2). A pSYM with multiple POI's is an example of ‘gene stacking’ (FIG. 10) can also be employed. Instances where different symbiont forming inoculums (having the same or different POIs) are use on the same host plant is an example of ‘symbiont stacking’ to give a plant the benefit of multiple POIs per plant.

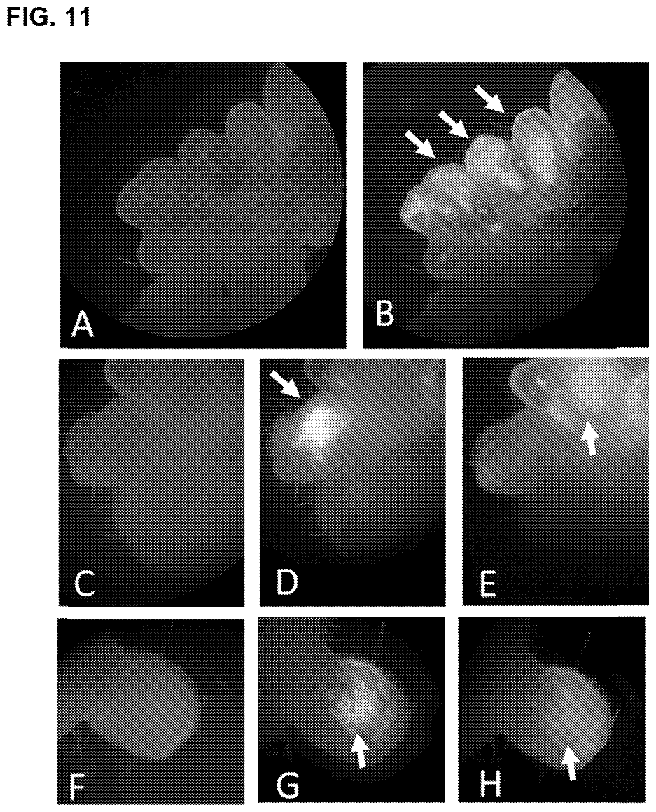

In this example, we used Agrobacterium-mediated transformation with co-inoculation of separate Agrobacterium symbiont forming inoculums, one having a unique pSYM plasmid encoding for GFP and the other with a pSYM plasmid encoding for mCherry. Detection of GFP and mCherrry accumulation was done by fluorescence microscopy examination (FIG. 11). Symbiont living cells were utilized to track the localization and dynamics of proteins, and sections of symbiont were analyzed under microscope to detect cells expressing GFP, cells expressing mCherry, and cells with both GFP and mCherry expression. This example demonstrated the versatility of symbiont cells expressing unique POIs in different cells (FIG. 11, panels B-E) or multiple POIs in the same cells (FIG. 11, panels G-H) and their ability to be supported on the same host plant.

### Example 5. Production and Export of the POI Product

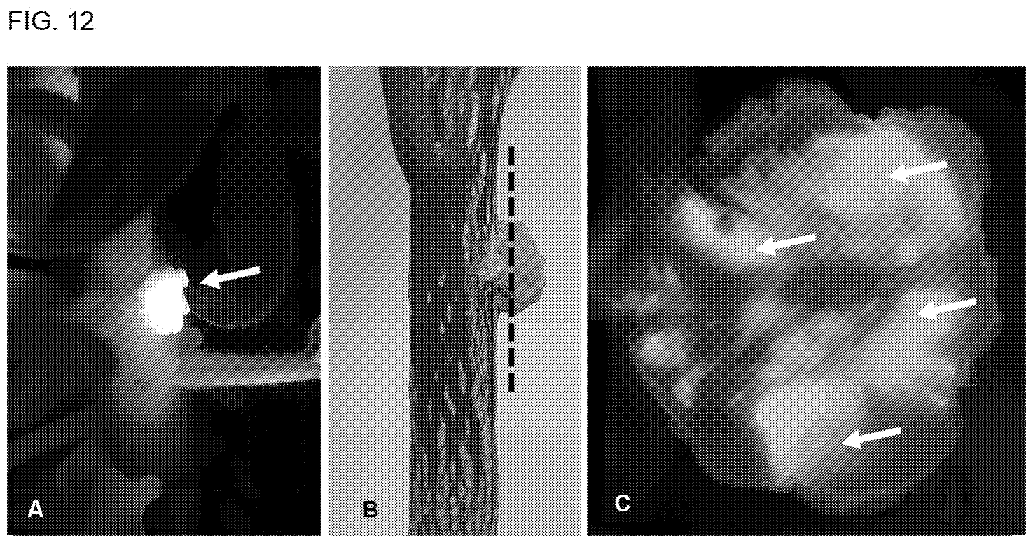

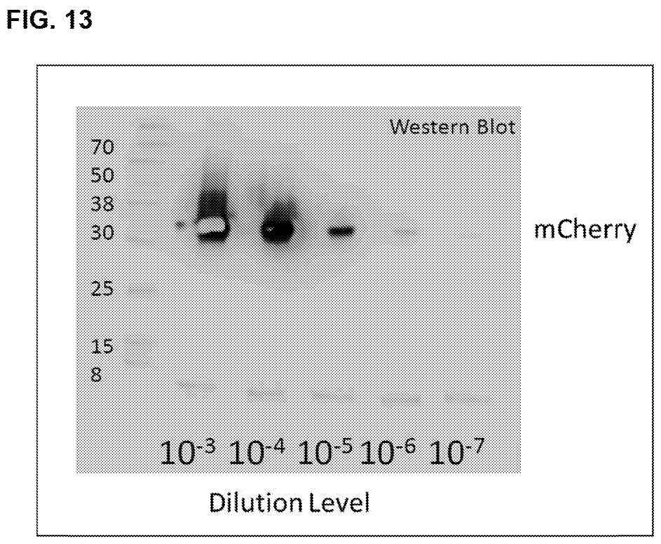

Symbionts can produce and accumulate large amounts of a desired POI product (e.g. protein, FIG. 12). Preliminary evaluations suggest up to 30% of the symbiont tissue may be POI product (FIG. 13). Symbionts expressing GFP were generated on tomato and citrus host plants using Agrobacterium spp. single inoculation (e.g., single strain) (FIG. 12). Combining both GFP and mCherry allowed the visualization and quantification of gene expression by measuring fluorescent intensity and protein accumulation using western blot (FIG. 13). To extract total protein from the symbiont, 1 g of symbiont material was used and turned into powder by freezing with liquid nitrogen and pulverizing the tissue. This was then suspended in a protein extraction buffer (for example, 150 mM Tris-HCl, pH 7.5; 150 mM NaCl; 5 mM EDTA; 1% IGEPAL® CA-630; and a 1% (vol/vol) protease inhibitor mixture 1 tablet 100 mL). For extraction, the buffer was added at 2 mL/g of tissue powder. Samples were clarified by 20 minutes centrifugations at 4° C. The supernatant was collected and several dilutions were made to 10⁷ dilution for input and analyzed under reducing conditions on an SDS-PAGE gel. The samples were then blotted onto a nitrocellulose membrane and incubated with antibodies according to the manufacturer's protocol (ThermoFisher®). Membranes were incubated using a chemiluminescent substrate and imaging and data was captured (FIG. 13).

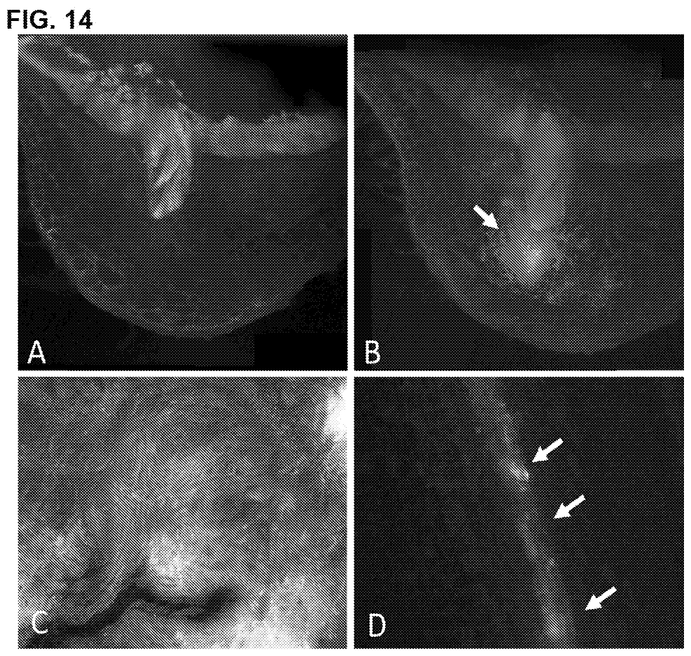

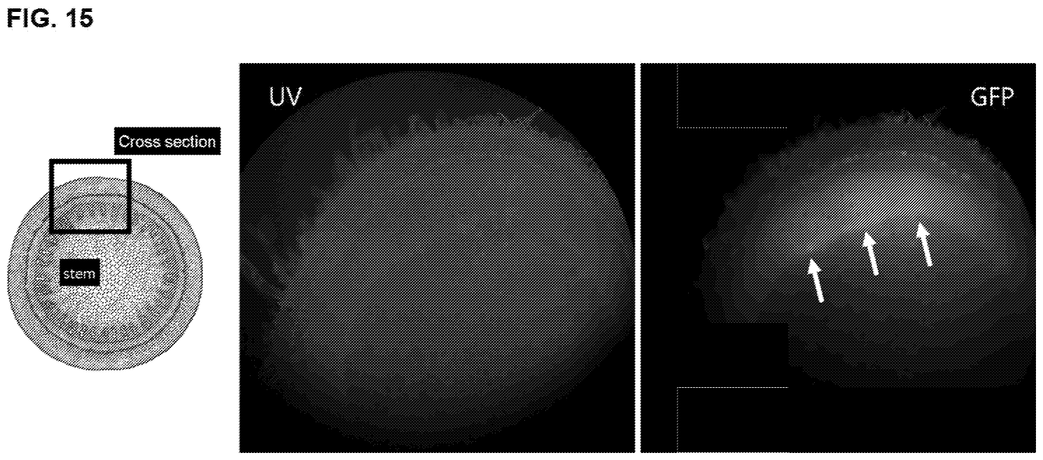

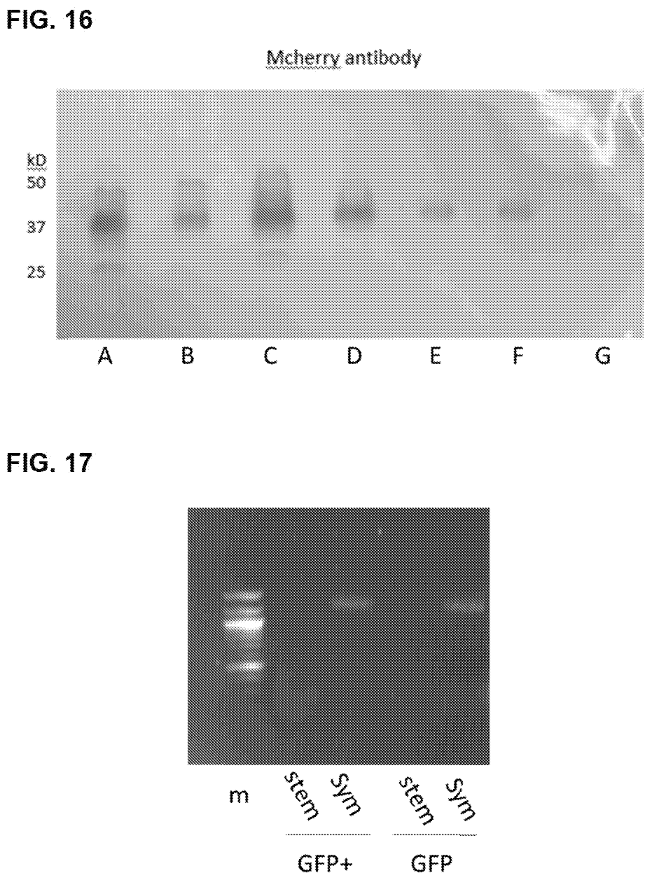

A symbiont can induce formation of a sophisticated vascular network connection with the host plant consisting of water-conducting vessels and assimilate-transporting sieve elements (FIG. 14, panels A and B). Symbiont cells are tightly connected by functioning plasmodesmata. Toluidine blue was used to distinguish between phloem and xylem cells since cells found in phloem have primary cell walls only while cells found in xylem have both primary and secondary cell walls (FIG. 14, panel C). High-level of POI expression in the symbiont cells in combination and the high amount of vascularized tissues facilitate the movement of the POI into host plant vascular tissue. Fluorescent proteins GFP and mCherry were used to detect and monitor using a fluorescence microscope the accumulation and movement of the protein from symbiont cells to plant vascular system in a tomato plant. Host plant tissues, 1-2 cm above the symbiont, were collected with longitudinal (FIG. 14, panel D) and cross sections (FIG. 15) to confirm GFP/mCherry movement (by fluorescence microscopy). Western blot techniques were also used to detect and analyze proteins accumulation in the symbiont and in the plant host stem validating the results from microscopy analyses (FIG. 16).

Solutes enter the symbiont via vascular tissue, which is connected to that of the host plant and consists of phloem for the transport of assimilates and xylem for water and minerals, in the same way the products of the symbiont is transported out from the symbiont to the host plant. While the product is capable of moving from the symbiont cells to the host plant, there is no genetic material movement from the symbiont to the host plant cell. We verified that the DNA of the POI is restricted to the symbiont by using PCR detection using specific primers to the POI and we examined the symbiont and neighboring stem sections. PCR analyses of symbiont and host plant tissue showed that only symbiont cells are genetically transformed with the POI (FIG. 17) indicating that the host plant is not transformed with the POI. This provides the host plant with a new characteristic but without genetic modification.

### Example 6: Effect of POI on the Host Plant

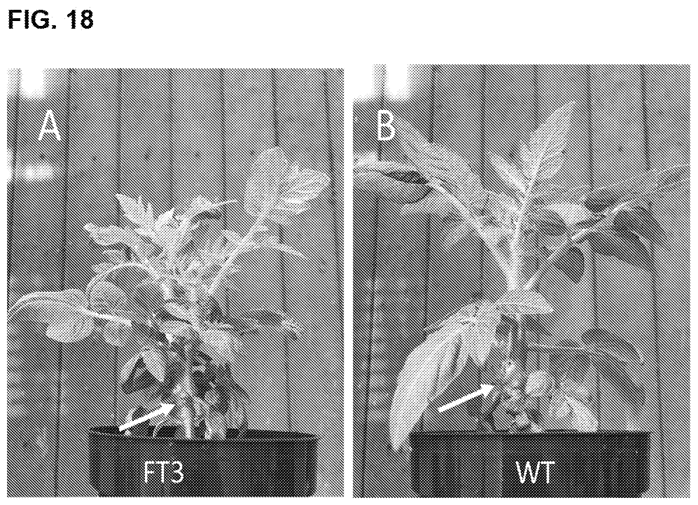

Symbiont tissue is highly versatile and it can adapt or be adapted to many different functions or activities. For example, FLOWERING LOCUS T (FT3) protein is synthesized in the leaf and translocated via the phloem and through the graft unions to control flowering in plants, and its overexpression is often associated with plant dwarfing. We generated a symbiont on tomato plant using Agrobacterium tumefaciens with pSYM to deliver the PHG and FT3 products to the plant (FIG. 18, panel A), and also generated a symbiont on tomato using wild type Agrobacterium tumefaciens (i.e. lacking pSYM) as a control (FIG. 18, panel B).

Tomato plants with symbionts expressing FT3 were bushy, having many branches and leafy structures compared to the control (compare FIG. 18, panels A and B). The symbiont expressing FT3 increased the number of branches (FIG. 18, panel A) modifying the tomato phyllotaxy where only one leaf is present at each node and the central stem of the plant is dominant over other side stems as shown on the control tomato plant (FIG. 18, panel B).

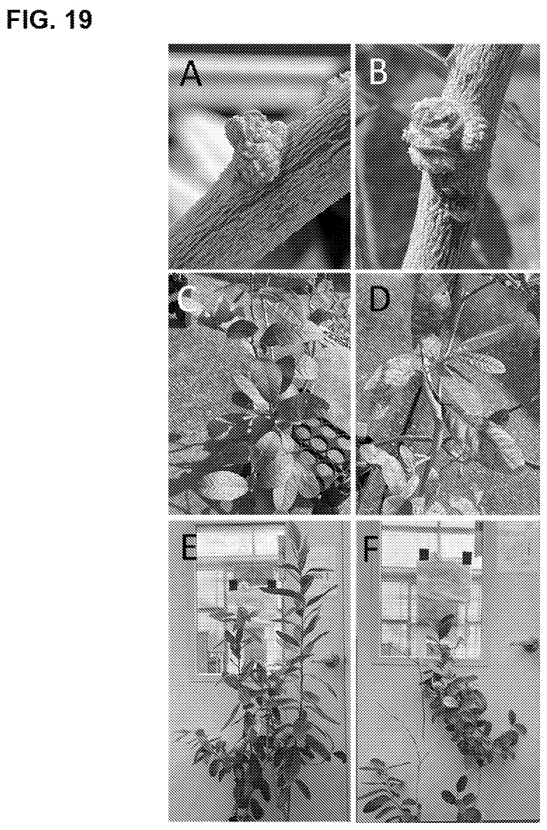

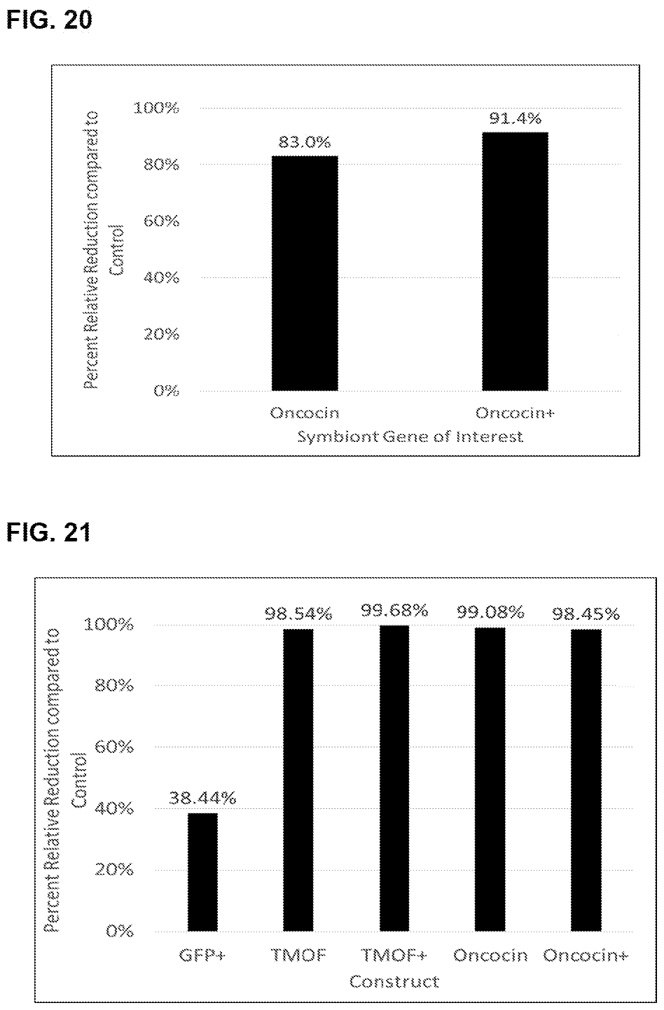

Symbionts can also be used to modify and modulate plant phenotype, enhancing resistance for a specific pathogen to improve defense mechanisms, and increase plant fitness. FIG. 19 shows an example of enhancing a plant's resistance to a specific pathogen. Here symbionts were generated on citrus using Agrobacterium tumefaciens comprising pSYM having PHG and oncocin (an antimicrobial peptide) thereby generating symbionts comprising PHG and oncocin (FIG. 19, panel A). As a control symbionts were generated on citrus with wild type A. tumefaciens (FIG. 19B) as a “no oncocin” control. The oncocin producing symbiont is designed to transfer oncocin to treat/kill Candidatus Liberibacter asiaticus (CLas). CLas is the causal agent of Huanglongbing (a.k.a. citrus greening disease) which causes devastating yield losses in citrus worldwide. To date, there is no established cure for this disease. We took advantage of the symbiont structure which is highly vascularized to produce and deliver this antibacterial peptide to the host plant and against CLas bacteria. To improve the export of the peptide it was fused to a signal/target sequence peptide (SS or +). Signal sequences are found in proteins that are targeted, for example, to the endoplasmic reticulum and eventually destined to be secreted extracellularly. The symbionts expressing “oncocin” and “oncocin+” both reduced the CLas titer (FIG. 20) over time as determined by qPCR, and also the plant health improved as indicated by the reduction of typical HLB plant symptoms (FIG. 19, panels C and E) including reduction of blotchy mottle compared to the control (FIG. 19, panels D and F).

As shown, symbionts can be used to express and translocate products to directly interfere with infection or kill pathogens present in the host plant. Symbionts expressing Oncocin and Oncocin+, were previously identified as improving citrus health and reducing CLas bacteria titer (as described, FIG. 19 and FIG. 20). To further investigate this, we studied CLas positive citrus host plants with different symbionts expressing different POIs (GFP+, TMOF, TMOF+, Oncocin and Oncocin+) and monitored CLas titer and efficacy of these different POI by qPCR analyses of CLas titer effects (FIG. 21). Symbiont expressing “GFP+” (GFP with signal sequence) was used as a control. The results indicated that both TMOF, TMOF+, Oncocin, and Oncocin+have an antibacterial effect on CLas by reducing its titer (FIG. 21).

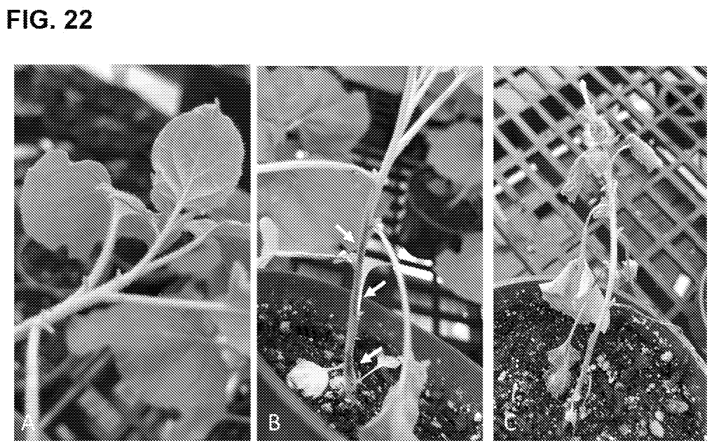

As shown, the versatility of symbionts of this invention provides the ability to improve host plant characteristics and control plant pests. As another example, symbionts can be used to generate an adverse plant effect that could be used as an herbicide by, for example, triggering plant hypersensitivity response and cell death. As an example, Nicotiana benthamiana was injected with a symbiont forming inoculum with a POI for an effector protein from CLas that is recognized by plant nucleotide-binding, leucine-rich repeat (NLR) immune receptors causing excessive production of reactive oxygen species (ROS) which leads to activation of cell death processes and kills the host plant (FIG. 22).

The foregoing is illustrative of the present invention, and is not to be construed as limiting thereof. Additional variations of the embodiments discussed above will be appreciated by those skilled in the art. Therefore, the above-described embodiments should be regarded as illustrative rather than restrictive. Accordingly, it should be appreciated that variations to those embodiments can be made by those skilled in the art without departing from the scope of the invention as defined by the following claims, with equivalents of the claims to be included therein.

That which is claimed is:

1. A symbiont forming inoculum comprising a polynucleotide encoding a phytohormone biosynthetic enzyme and a polynucleotide of interest, wherein the phytohormone biosynthetic enzyme is at least one cytokinin biosynthetic enzyme and/or an auxin biosynthetic enzyme.

2. The symbiont forming inoculum of claim 1, wherein the polynucleotide encoding a phytohormone biosynthetic enzyme and the polynucleotide of interest are comprised in a cell, optionally wherein the cell is a plant cell or a bacterial cell.

3. The symbiont forming inoculum of claim 1 or claim 2, wherein the phytohormone biosynthetic enzyme is from a bacterial species and/or a plant species.

4. The symbiont forming inoculum of any one of claims 1-3, wherein the phytohormone biosynthetic enzyme is an indole-3-acetamide hydrolase (iaaH) (E.C. Number: EC 3.5.1.4), amidase 1 (EC 3.5.1.4), a tryptophan 2-monooxygenase (IaaM) (EC 1.13.12.3), an indole-3-lactate synthase (EC 1.1.1.110), a L-tryptophan-pyruvate aminotransferase 1 (EC 2.6.1.99), a tryptophan aminotransferase-related protein 1 (EC 2.6.1.27), indole-3-acetaldehyde oxidase (EC 1.2.3.7), a tryptophan decarboxylase 1/tryptophan decarboxylase 2 (EC4.1.1.105), an isopentenyl transferase (Ipt) and/or a Tzs (EC 2.5.1.27).

5. The symbiont forming inoculum of any one of claims 1-4, wherein the phytohormone biosynthetic enzyme is an indole-3-acetamide hydrolase (iaaH), a tryptophan 2-monooxygenase (IaaM), and/or an isopentenyl transferase (Ipt).

6. The symbiont forming inoculum of any one of claims 1-5, wherein the phytohormone biosynthetic enzyme is indole-3-lactate synthase.

7. The symbiont forming inoculum of any one of claims 1-6, further comprising a polynucleotide encoding a plast polypeptide (e.g., plasticity polypeptide), optionally wherein the plast polypeptide is 6b, rolB, rolC, and/or orf13.

8. The symbiont forming inoculum of any one of claims 1-7, wherein the polynucleotide encoding a phytohormone biosynthetic enzyme and the polynucleotide of interest are comprised in a single nucleic acid construct or in two or more nucleic acid constructs (e.g., one or more expression cassettes).

9. The symbiont forming inoculum of claim 7 or claim 8, wherein the polynucleotide encoding a plast polypeptide is comprised in a nucleic acid construct, optionally wherein the polynucleotide encoding a plast polypeptide is in the same or a separate nucleic acid construct (e.g., expression cassette) as/from the polynucleotide encoding a phytohormone biosynthetic enzyme and/or the polynucleotide of interest.

10. The symbiont forming inoculum of any one of claim 8 or claim 9, wherein the one or more nucleic acid constructs are comprised in one or more vectors.

11. The symbiont forming inoculum of claim 10, wherein the one or more vectors are a plasmid, a T-DNA, a bacterial artificial chromosome, viral vector, or a binary-bacterial artificial chromosome.

12. The symbiont forming inoculum of any one of claims 1-11, wherein the polynucleotide of interest encodes a biomolecule, a bioactive molecule and/or a polypeptide involved in the biosynthesis of a bioactive molecule.

13. The symbiont forming inoculum of claim 12, wherein expression of the polynucleotide of interest confers increased abiotic stress resistance (e.g., high salt tolerance, high heat tolerance, heavy metals tolerance, cold tolerance, drought tolerance, excessive water tolerance, tolerance to UV radiation), increased resistance or tolerance to a pathogen (e.g., viral, fungal, bacterial) or pest (e.g., insect, nematode), or increased tolerance to an herbicide,

14. The symbiont forming inoculum of claim 12 or claim 13, wherein the bioactive molecule is a biostimulant, a biofungicide, a bioherbicide, an insecticidal protein/peptide (e.g., a bioinsecticide; e.g., jaburetox (peptide JBTX; a bioinsecticide derived from Jack beans (Canavalia ensiformis seeds)); insecticidal peptides from spider venom (e.g., from Hadronyche versuta); trypsin modulating oostatic factor (TMOF); Bacillus thuringiensis toxins (δ endotoxins, e.g., Cry toxin, Cyt toxin); a vegetative insecticidal protein (Vip)), a nutrient (e.g., nitrogen, e.g., a leghemoglobin, a nitrogenase), a plant growth regulator (auxin, cytokinin, gibberellin, ethylene; growth inhibitor/retardant), a plant lipid, a plant fatty acid, a plant oil, an RNA (e.g., siRNA, dsRNA, miRNA, shRNA), a plantibody, a stylet sheath inhibitory protein (e.g., ficin, bromelain), a ribozyme, a bacteriocin, an antimicrobial peptide (e.g., oncocin), an aptamer, a nuclease, a zinc finger nuclease (ZFN), a Transcription Activator-Like Effector Nuclease (TALEN) and/or an engineered meganuclease.

14. The symbiont forming inoculum of any one of claims 1-13, wherein the polynucleotide of interest encodes a polypeptide operably linked to a targeting sequence, optionally wherein the targeting sequence locates the protein to a membrane, a subcellular location or an extracellular location.

15. The symbiont forming inoculum of claim 14, wherein the targeting sequence is a membrane targeting sequence, an endoplasmic reticulum targeting sequence, a mitochondrial targeting sequence, a chloroplast targeting sequence, nuclear targeting sequence, vacuolar targeting sequence, peroxisomal targeting sequence, lysosomal targeting sequence, or a plant virus movement protein.

16. The symbiont forming inoculum of any one of claims 1-15, wherein the polynucleotide encoding a phytohormone biosynthetic enzyme (e.g., the polynucleotide encoding iaaH, the polynucleotide encoding IaaM, and/or the polynucleotide encoding isopentenyl transferase (Ipt), and/or the polynucleotide encoding indole-3-lactate synthase) and/or the polynucleotide encoding a plast polypeptide is/are operably linked to a nuclear targeting (nuclear localization) sequence.

17. The symbiont forming inoculum of any one of claims 1-16, wherein the polynucleotide encoding a phytohormone biosynthetic enzyme and the polynucleotide of interest are each operably linked to a single promoter or to at least two separate promoters, in any combination.

18. The symbiont forming inoculum of any one of claims 1-17, wherein when the polynucleotide encoding a phytohormone biosynthetic enzyme encodes iaaH, IaaM and Ipt, the polynucleotide(s) encoding iaaH, IaaM and Ipt is operably linked to a single promoter and the polynucleotide of interest is operably linked to the single promoter or to a separate promoter.

19. The symbiont forming inoculum of any one of claims 7-18, wherein the polynucleotide encoding a plast polypeptide is operably linked to a promoter, optionally the polynucleotide encoding a plast polypeptide is operably linked to the same promoter or a separate promoter as that which is operably linked to the polynucleotide encoding a phytohormone biosynthetic enzyme and/or the polynucleotide of interest.

20. The symbiont forming inoculum of any one of claims 17-19, wherein the single promoter, the separate promoter, and/or the two or more separate promoters are each a constitutive promoter or an inducible promoter in any combination.

21. The symbiont forming inoculum of any one of claims 1-20, wherein the polynucleotide of interest is expressed in the symbiont forming inoculum.

22. The symbiont forming inoculum of any one of claims 2-21, wherein the bacterial cell comprises a Type IV Secretion System (T4SS, e.g., T4ASS, (e.g., VirB/D4 system), T4BSS) or a Type III Secretion System (T3SS).

23. The symbiont forming inoculum of any one of claims 2-22, wherein the bacterial cell is from a bacterial genera of Agrobacterium spp. (e.g., A. tumefaciens (e.g., biovar 1), A. rhizogenes (e.g., biovar 2), A. vitis (e.g., biovar 3), A. fabrum (e.g., strain C58), Rhizobium spp., Mesorhizobium spp., Sinorhizobium spp., Bradyrhizobium spp., Pseudomonas spp. (e.g., P. savastanoi pv. Savastanoi), Phyllobacterium spp., Ochrobactrum spp., Azobacter spp., Closterium spp., Klebsiella spp., Rhodospirillum spp., or Xanthomonas spp.

24. The symbiont forming inoculum of any one of claims 2-21, wherein the symbiont forming inoculum comprised in a cell comprises two or more cells in the form of cell culture, a plant callus, a callus culture, and/or a suspension culture.

25. The symbiont forming inoculum of any one of claims 2-21 or 24, wherein the plant cell is from a macroalgae, an angiosperm, a gymnosperm or a pteridophyte.

26. A symbiont comprising a plant cell comprising and expressing a polynucleotide encoding a phytohormone biosynthetic enzyme and a polynucleotide of interest, wherein the phytohormone biosynthetic enzyme is at least one cytokinin biosynthetic enzyme and/or at least one auxin biosynthetic enzyme and the plant cell of the symbiont autonomously divides.

27. The symbiont of claim 26, wherein the plant cell comprises more than one plant cell and forms an undifferentiated multi-cellular structure when transplanted onto a plant or part thereof.

28. The symbiont of claim 26 or claim 27, wherein the phytohormone biosynthetic enzyme is from a bacterial species and/or a plant species.

29. The symbiont of any one of claims 26-28, wherein the phytohormone biosynthetic enzyme is an indole-3-acetamide hydrolase (iaaH) (E.C. Number: EC 3.5.1.4), amidase 1 (EC 3.5.1.4), a tryptophan 2-monooxygenase (IaaM) (EC 1.13.12.3), an indole-3-lactate synthase (EC 1.1.1.110), a L-tryptophan-pyruvate aminotransferase 1 (EC 2.6.1.99), a tryptophan aminotransferase-related protein 1 (EC 2.6.1.27), indole-3-acetaldehyde oxidase (EC 1.2.3.7), a tryptophan decarboxylase 1/tryptophan decarboxylase 2 (EC4.1.1.105), an isopentenyl transferase (Ipt) and/or a Tzs (EC 2.5.1.27).

30. The symbiont of any one of claims 26-29, wherein the phytohormone biosynthetic enzyme comprises indole-3-acetamide hydrolase (iaaH), tryptophan 2-monooxygenase (IaaM), and/or isopentenyl transferase (Ipt), optionally wherein the phytohormone biosynthetic enzyme comprises indole-3-lactate synthase.

31. The symbiont of any one of claims 26-30, further comprising a polynucleotide encoding a plast polypeptide (e.g., plasticity polypeptide), optionally wherein the plast polypeptide is 6b, rolB, rolC, and/or orf13.

32. The symbiont of any one of claims 26-31, wherein the polynucleotide encoding a phytohormone biosynthetic enzyme and the polynucleotide of interest are comprised in a single nucleic acid construct or in two or more nucleic acid constructs (e.g., one or more expression cassettes).

33. The symbiont of claim 31 or claim 32, wherein the polynucleotide encoding a plast polypeptide is comprised in a nucleic acid construct, optionally wherein the polynucleotide encoding a plast polypeptide is in the same or a separate nucleic acid construct as the polynucleotide encoding a phytohormone biosynthetic enzyme and/or the polynucleotide of interest.

34. The symbiont of any one of claims 26-33, wherein the polynucleotide of interest encodes a biomolecule, a bioactive molecule and/or a polypeptide involved in the biosynthesis of a bioactive molecule.

35. The symbiont of claim 34, wherein expression of the polynucleotide of interest confers increased abiotic stress resistance (e.g., high salt tolerance, high heat tolerance, heavy metals tolerance, cold tolerance, drought tolerance, excessive water tolerance, tolerance to UV radiation), increased resistance or tolerance to a pathogen (e.g., viral, fungal, bacterial) or pest (e.g., insect, nematode), or increased tolerance to an herbicide.

36. The symbiont of claim 34 or claim 35, wherein the bioactive molecule is a pharmaceutical, a biostimulant, a biofungicide, a bioherbicide, an insecticidal protein/peptide (e.g., a bioinsecticide, e.g., jaburetox (peptide JBTX), insecticidal peptides from spider venom (e.g., from Hadronyche versuta); trypsin modulating oostatic factor (TMOF), Bacillus thuringiensis toxins (δ endotoxins, e.g., Cry toxin, Cyt toxin), a vegetative insecticidal protein (Vip)), a nutrient (e.g., nitrogen, e.g., a leghemoglobin, a nitrogenase), a plant growth regulator (auxin, cytokinin, gibberellin, ethylene; growth inhibitor/retardant), an RNA (e.g., siRNA, dsRNA, miRNA, shRNA), a plantibody, a stylet sheath inhibitory protein (e.g., ficin, bromelain), a ribozyme, a bacteriocin, a plant lipid, a plant fatty acid, a plant oil, an antimicrobial peptide (e.g., oncocin), an aptamer, a nuclease, a zinc finger nuclease (ZFN), a Transcription Activator-Like Effector Nuclease (TALEN) and/or an engineered meganuclease.

37. The symbiont of any one of claims 26-36, wherein the polynucleotide of interest encodes a polypeptide operably linked to a targeting sequence, optionally wherein the targeting sequence locates the polypeptide to a membrane, a subcellular location or an extracellular location.

38. The symbiont of claim 37, wherein the targeting sequence is a membrane targeting sequence, an endoplasmic reticulum targeting sequence, a mitochondrial targeting sequence, a chloroplast targeting sequence or a plant virus movement protein.

39. The symbiont of any one of claims 26-38, wherein the polynucleotide encoding a phytohormone biosynthetic enzyme (e.g., a polynucleotide encoding iaaH, a polynucleotide encoding IaaM, a polynucleotide encoding isopentenyl transferase (Ipt), and/or a polynucleotide encoding indole-3-lactate synthase) and/or the polynucleotide encoding a plast polypeptide is/are operably linked to a nuclear targeting sequence.

40. The symbiont of any one of claims 26-39, wherein the polynucleotide encoding a phytohormone biosynthetic enzyme and the polynucleotide of interest are operably linked to a single promoter or to at least two separate promoters, in any combination.

41. The symbiont of any one of claims 26-40, wherein when the polynucleotide encoding a phytohormone biosynthetic enzyme encodes iaaH, IaaM, and Ipt, the polynucleotide(s) encoding iaaH, IaaM, and Ipt are operably linked to a single promoter and the polynucleotide of interest is operably linked to the same promoter or a separate promoter.

42. The symbiont of any one of claims 26-41, wherein the polynucleotide encoding a plast polypeptide is operably linked to a promoter, optionally the polynucleotide encoding a plast polypeptide is operably linked to the same promoter or a separate promoter as that which is operably linked to the polynucleotide encoding a phytohormone biosynthetic enzyme and/or the polynucleotide of interest.

42. The symbiont of any one of claims 26-42, wherein the plant cell is from a macroalgae, an angiosperm, a gymnosperm or a pteridophyte.

43. A host plant comprising at least one symbiont of any one of claims 26-42, wherein the at least one symbiont is located on at least one site on the host plant.

44. The host plant of claim 43, wherein the at least one site on the host plant is on an explant, embryo, leaf, shoot, stem, branch, kernel, ear, cob, husk, stalk, epidermal tissue, apical meristem tissue, floral tissue (e.g., pollen, pistil, ovule, anther, stamen, corolla, sepal, petal, receptacle, filament, style, stigma, etc), fruit, seed, pod, capsule, cotyledon, hypocotyl, petiole, tuber, corm, root, root tip, symbiont, burl, plant food body, dormatia, extrafloral nectary, nodule, plant neoplasm or gall

45. The host plant of any one of claim 43 or claim 44, wherein the polynucleotide of interest is expressed in the symbiont and an expression product of the polynucleotide of interest and/or a product made using the expression product of the polynucleotide of interest is transported into the host plant.

46. The host plant of any one of claims 43-45, wherein the host plant is from a wild type plant of any age or size (e.g., seedling, juvenile plant, or mature plant).

47. The host plant of any one of claims 43-46, wherein the host plant is a macroalgae, an angiosperm plant, a gymnosperm plant or a pteridophyte plant.

48. A method of producing a symbiont forming inoculum, the method comprising:
introducing into a cell a polynucleotide encoding a phytohormone biosynthetic enzyme and a polynucleotide of interest or introducing a polynucleotide encoding a phytohormone biosynthetic enzyme into a transgenic cell that comprises a polynucleotide of interest, wherein the phytohormone biosynthetic enzyme is at least one cytokinin biosynthetic enzyme and/or an auxin biosynthetic enzyme, thereby producing the symbiont forming inoculum, optionally, wherein the cell is a plant cell or a bacterial cell.

49. The method of claim 48, further comprising culturing the cell to produce a population of cells comprising the polynucleotide encoding a phytohormone biosynthetic enzyme and the polynucleotide of interest.

50. A method of producing a symbiont forming inoculum, the method comprising
(a) (i) introducing into/onto at least one site on a plant (or a part thereof (e.g., explant)) a polynucleotide encoding a phytohormone biosynthetic enzyme and a polynucleotide sequence of interest or transplanting a plant cell comprising the same or inoculating a bacterial cell comprising the same onto at least one site on the plant (or a part thereof), or
(ii) introducing a polynucleotide encoding a phytohormone biosynthetic enzyme, or transplanting a plant cell comprising the same, or inoculating a bacterial cell comprising the same onto at least one site on the plant (or a part thereof) into/onto at least one site on a plant (or a part thereof), the plant (or a part thereof) of (ii) comprising a polynucleotide sequence of interest,
wherein the phytohormone biosynthetic enzyme is at least one cytokinin biosynthetic enzyme and/or an auxin biosynthetic enzyme, thereby producing a symbiont on the plant (or part thereof) that comprises the polynucleotide encoding a phytohormone biosynthetic enzyme and the polynucleotide sequence of interest; and
(b) selecting one or more cells from the symbiont on the plant, to provide one or more cells comprising the polynucleotide encoding a phytohormone biosynthetic enzyme and the polynucleotide sequence of interest, thereby producing the symbiont forming inoculum.

51. The method of claim 50, further comprising (c) culturing the one or more cells from (b) to produce a population of plant cells (e.g., a callus culture and/or a suspension culture) comprising the polynucleotide encoding a phytohormone biosynthetic enzyme and the polynucleotide sequence of interest.

52. The method of claim 50 or claim 51, wherein the phytohormone biosynthetic enzyme is from a bacterial species and/or a plant species.

53. The method of any one of claims 50-52, wherein the phytohormone biosynthetic enzyme is an indole-3-acetamide hydrolase (iaaH) (E.C. Number: EC 3.5.1.4), amidase 1 (EC 3.5.1.4), a tryptophan 2-monooxygenase (IaaM) (EC 1.13.12.3), an indole-3-lactate synthase (EC 1.1.1.110), a L-tryptophan-pyruvate aminotransferase 1 (EC 2.6.1.99), a tryptophan aminotransferase-related protein 1 (EC 2.6.1.27), indole-3-acetaldehyde oxidase (EC 1.2.3.7), a tryptophan decarboxylase 1/tryptophan decarboxylase 2 (EC4.1.1.105), an isopentenyl transferase (Ipt) and/or a Tzs (EC 2.5.1.27).

54. The method of any one of claims 50-53, wherein the phytohormone biosynthetic enzyme comprises indole-3-acetamide hydrolase (iaaH), tryptophan 2-monooxygenase (IaaM), and/or isopentenyl transferase (Ipt), and/or optionally comprises indole-3-lactate synthase.

55. The method of any one of claims 50-54, wherein the at least one site on the plant is on an above ground part of the plant and/or a below ground part of the plant.

56. The method of any one of claims 50-55, further comprising introducing into the cell or at least one site on a plant a polynucleotide encoding a plast polypeptide (e.g., a plasticity polypeptide), optionally wherein the plast polypeptide is 6b, rolB, rolC, and/or orf13.

57. The method of any one of claims 50-56, wherein the polynucleotide encoding a phytohormone biosynthetic enzyme and the polynucleotide of interest are introduced in a single nucleic acid construct or separately in two or more nucleic acid constructs (e.g., one or more expression cassettes).

58. The method of claim 56 or claim 57, wherein the polynucleotide encoding a plast polypeptide is comprised in a nucleic acid construct, optionally wherein the polynucleotide encoding the plast polypeptide is introduced in the same or a separate nucleic acid construct as the polynucleotide encoding a phytohormone biosynthetic enzyme and/or the polynucleotide of interest.

59. The method of claim 57 or claim 58, wherein the two or more nucleic acid constructs are comprised in one or more vectors, optionally wherein the vector is a plasmid, a T-DNA, a bacterial artificial chromosome, viral vector, or a binary-bacterial artificial chromosome.

60. The method of any one of claims 50-59, wherein the polynucleotide of interest encodes a biomolecule, a bioactive molecule and/or a polypeptide involved in the biosynthesis of a bioactive molecule.

61. The method of claim 60, wherein expression of the polynucleotide of interest confers increased abiotic stress resistance (e.g., high salt tolerance, high heat tolerance, heavy metals tolerance, cold tolerance, drought tolerance, excessive water tolerance, tolerance to UV radiation), increased resistance or tolerance to a pathogen (e.g., viral, fungal, bacterial) or pest (e.g., insect, nematode), and/or increased tolerance to an herbicide.

62. The method of claim 60 or claim 61, wherein the bioactive molecule is a biostimulant, a biofungicide, a bioherbicide, an insecticidal protein/peptide (e.g., a bioinsecticide; e.g., jaburetox (peptide JBTX; a bioinsecticide derived from Jack beans (Canavalia ensiformis seeds)); insecticidal peptides from spider venom (e.g., from Hadronyche versuta); trypsin modulating oostatic factor (TMOF); Bacillus thuringiensis toxins (δ endotoxins, e.g., Cry toxin, Cyt toxin); a vegetative insecticidal protein (Vip)), a nutrient (e.g., nitrogen, e.g., a leghemoglobin, a nitrogenase), a plant growth regulator (auxin, cytokinin, gibberellin, ethylene; growth inhibitor/retardant), an RNA (e.g., siRNA, dsRNA, miRNA, shRNA), a plantibody, a stylet sheath inhibitory protein (e.g., ficin, bromelain), a ribozyme, a bacteriocin, an antimicrobial peptide (e.g., oncocin), a plant lipid, a plant fatty acid, a plant oil, an aptamer, a nuclease, a zinc finger nuclease (ZFN), a Transcription Activator-Like Effector Nuclease (TALEN) and/or an engineered meganuclease.

63. The method of any one of claims 50-62, wherein the polynucleotide of interest encodes a polypeptide operably linked to a targeting sequence, optionally wherein the targeting sequence locates the protein to a membrane, a subcellular location or an extracellular location, optionally wherein the targeting sequence is a membrane targeting sequence, an endoplasmic reticulum targeting sequence, a mitochondrial targeting sequence, a chloroplast targeting sequence or a plant virus movement protein.

64. The method of any one of claims 50-63, wherein the polynucleotide encoding a phytohormone biosynthetic enzyme and/or the polynucleotide encoding a plast polypeptide is/are operably linked to a nuclear targeting sequence.

65. The method of any one of claims 50-64, wherein the polynucleotide encoding a phytohormone biosynthetic enzyme and the polynucleotide of interest are each operably linked to a single promoter or to at least two separate promoters, in any combination.

66. The method of any one of claims 50-65, wherein when the polynucleotide encoding a phytohormone biosynthetic enzyme encodes iaaH, IaaM, and Ipt, the polynucleotide(s) encoding iaaH, IaaM, and Ipt are operably linked to a single promoter and the polynucleotide of interest is operably linked to the single promoter or to a separate promoter.

67. The method of any one of claims 50-66, wherein when the polynucleotide encoding a phytohormone biosynthetic enzyme comprises a polynucleotide encoding iaaH, a polynucleotide encoding IaaM, and a polynucleotide encoding Ipt, the polynucleotide encoding iaaH, the polynucleotide encoding IaaM, and the polynucleotide encoding Ipt are each operably linked to at least two separate promoters and the polynucleotide of interest is operably linked to a separate promoter from the at least two separate promoters or is linked to at least one of the at least two separate promoters.

68. The method of any one of claims 56-67, wherein the polynucleotide encoding a plast polypeptide is operably linked to a promoter, optionally the polynucleotide encoding a plast polypeptide is operably linked to the same promoter or a separate promoter as that which is operably linked to the polynucleotide encoding a phytohormone biosynthetic enzyme and/or the polynucleotide of interest.

69. The method of any one of claims 65-68, wherein the promoter, the single promoter and/or the two or more separate promoters are a constitutive promoter or an inducible promoter.

70. The method of any one of claims 48-54 or 56-69, wherein the bacterial cell comprises a Type IV Secretion System (T4SS, e.g., T4ASS, (e.g., VirB/D4 system), T4BSS) or a Type III Secretion System (T3SS).

71. The method of any one of claims 48-54 or 56-69, wherein the bacterial cell is from a bacterial genera of Agrobacterium spp. (e.g., A. tumefaciens (e.g., biovar 1), A. rhizogenes (e.g., biovar 2), A. vitis (e.g., biovar 3), A. fabrum (e.g., strain C58), Rhizobium spp., Mesorhizobium spp., Sinorhizobium spp., Bradyrhizobium spp., Pseudomonas spp. (e.g., P. savastanoi pv. Savastanoi), Phyllobacterium spp., Ochrobactrum spp., Azobacter spp., Closterium spp., Klebsiella spp., Rhodospirillum spp., or Xanthomonas spp.

72. The method of any one of claims 50-69, wherein the plant, plant part or plant cell is from a wild type plant or a transgenic plant of any age or size (e.g., seedling, juvenile plant, or mature plant).

73. The method of any one of claims 50-69 or 72, wherein the plant, plant part or plant cell is from a macroalgae, an angiosperm plant, a gymnosperm plant or a pteridophyte plant.

74. The method of any one of claims 50-69, 72 or 73, wherein the plant cell is from a plant cell culture (callus culture or suspension culture), a protoplast, seedling, explant, embryo, leaf, shoot, stem, branch, kernel, ear, cob, husk, stalk, epidermal tissue, apical meristem tissue, floral tissue (e.g., pollen, pistil, ovule, anther, stamen, corolla, sepal, petal, receptacle, filament, style, stigma, etc), fruit, seed, pod, capsule, cotyledon, hypocotyl, petiole, tuber, corm, root, root tip, symbiont, burl, plant food body, dormatia, extrafloral nectary, nodule, gall or plant neoplasm.

75. The method of any one of claims 50-69 or 72-74, wherein the at least one site on a plant is an explant, embryo, leaf, shoot, stem, branch, kernel, ear, cob, husk, stalk, epidermal tissue, apical meristem tissue, floral tissue (e.g., pollen, pistil, ovule, anther, stamen, corolla, sepal, petal, receptacle, filament, style, stigma, etc), fruit, seed, pod, capsule, cotyledon, hypocotyl, petiole, tuber, corm, root, root tip, symbiont, burl, plant food body, dormatia, extrafloral nectary, nodule, plant neoplasm or gall.

76. The method of any one of claims 50-75, wherein introducing is via bacterial mediated transformation, agroinfiltration, viral-mediated transformation, particle bombardment (biolistics), electroporation, microinjection, lipofection (liposome mediated transformation), sonication, silicon fiber mediated transformation, chemically stimulated DNA uptake (e.g., polyfection; e.g., polyethylene glycol (PEG) mediated transformation), and/or laser microbeam (UV) induced transformation.

77. The method of any one of claims 50-69 or 72-76, wherein (i) the polynucleotide encoding a phytohormone biosynthetic enzyme and a polynucleotide sequence of interest or (ii) the polynucleotide encoding a phytohormone biosynthetic enzyme is comprised in at least one plant cell, and the at least one plant cell is transplanted onto the at least one site on the plant.

78. The method of claim 77 wherein the plant is wounded at the at least one site prior to or during transplanting of the at least one plant cell to the at least one site on the plant.

79. The method of claim 76, wherein the introducing is via bacterial mediated transformation and comprises co-cultivating the plant cell or plant (or a part thereof, e.g., explant) with the cells of at least one bacterial species or strain, the bacterial cells comprising one or more of: the polynucleotide encoding a phytohormone biosynthetic enzyme, the polynucleotide of interest, and/or at least one polynucleotide encoding at least one plast polypeptide.

80. The method of claim 79, wherein the plant (or part thereof; e.g., explant) is wounded at the at least one site prior to or during co-cultivation with the cells of the at least one bacterial strain.

81. The method of claim 79 or claim 80, wherein the cells of the at least one bacterial species or strain comprise cells of at least two bacterial species or strains and the polynucleotide encoding the at least one phytohormone enzyme is comprised in a separate bacterial strain from the bacterial strain comprising the at least one polynucleotide of interest (e.g., dual bacterial transformation).

82. The method of any one of claims 79-81, wherein the at least one bacterial strain or species and/or the at least two bacterial strains or species is/are a bacterial cell comprising a Type IV Secretion System (T4SS, e.g., T4ASS, (e.g., VirB/D4 system), T4BSS) or a Type III Secretion System (T3SS).

83. The method of claim 82, wherein the bacterial cell is an Agrobacterium spp. cell (e.g., A. tumefaciens (e.g., biovar 1), A. rhizogenes (e.g., biovar 2), A. vitis (e.g., biovar 3), A. fabrum (e.g., strain C58), Rhizobium spp. cell, Mesorhizobium spp. cell, Sinorhizobium spp. cell, Bradyrhizobium spp. cell, Pseudomonas spp. cell (e.g., P. savastanoi pv. Savastanoi), Phyllobacterium spp. cell, Ochrobactrum spp. cell, Azobacter spp. cell, Closterium spp. cell, Kiebsiella spp. cell, Rhodospirillum spp. cell, or Xanthomonas spp. cell.

84. The method of any one of claims 48-83, the method further comprising editing of at least one nucleic acid in at least one cell of the symbiont forming inoculum to produce at least one edited nucleic acid, optionally wherein the editing is carried out with a gene editing nuclease.

85. The method of claim 84, wherein the at least one edited nucleic acid has modified expression (e.g., increased or decreased expression as compared to the same nucleic acid not so modified).

86. A symbiont forming inoculum produced by the method of any one of claims 48-85.

87. The symbiont forming inoculum of claim 86, wherein the inoculum comprises a bacterial cell, a bacterial culture, a plant cell, or a plant cell culture (e.g., a callus or a cell suspension).

88. A cell or protoplast from the symbiont forming inoculum of claim 86 or claim 87, wherein the cell or protoplast comprises the polynucleotide encoding a phytohormone biosynthetic enzyme and the polynucleotide of interest.

89. A method of modifying a host plant characteristic without modifying the plant genome, the method comprising
transplanting the symbiont forming inoculum of claim 86 or claim 87 or cell of claim 88, or the symbiont of any one of claims 26-42 to at least one site on a host plant; and
culturing the symbiont forming inoculum or symbiont at the at least one site on the host plant to form a symbiont on the host plant at the at least one site, wherein the polynucleotide of interest is expressed in the symbiont on the host plant and an expression product of the polynucleotide of interest and/or a product made using the expression product of the polynucleotide of interest is transported into the host plant, thereby producing a host plant having a modified characteristic.

90. The method of claim 89, wherein the polynucleotide of interest encodes a biomolecule, a bioactive molecule and/or a polypeptide involved in the biosynthesis of a bioactive molecule.

91. The method of claim 90, wherein expression of the polynucleotide of interest confers increased abiotic stress resistance (e.g., high salt tolerance, high heat tolerance, heavy metals tolerance, cold tolerance, drought tolerance, excessive water tolerance, tolerance to UV radiation), increased resistance or tolerance to a pathogen (e.g., viral, fungal, bacterial) or pest (e.g., insect, nematode), or increased tolerance to an herbicide

92. The method of claim 90 or claim 91, wherein the bioactive molecule is a pharmaceutical (e.g., a therapeutic protein, a therapeutic polynucleotide, a therapeutic chemical; e.g., a vaccine, an antibody, a recombinant antibody, an antibody fragment, a fusion protein, an antibody fusion protein, human serum albumin, gastric lipase, insulin, glucocerebrosidase, growth factor, a cytokine, hepatitis B surface antigen (HBsAg)), Apo-A1, alpha-galactosidase (PRX-102), alpha-galactosidase (PRX-102), acetylcholineesterase (PRX-105), antitumor necrosis factor (Pr-anti-TNF), IgG, interferon-alpha, plasmin, lactoferrin, lysozyme, and collagen), a biostimulant, a biofungicide, a bioherbicide, an insecticidal protein/peptide (e.g., a bioinsecticide; e.g., jaburetox (peptide JBTX; a bioinsecticide derived from Jack beans (Canavalia ensiformis seeds), insecticidal peptides from spider venom (e.g., from Hadronyche versuta); trypsin modulating oostatic factor (TMOF), Bacillus thuringiensis toxins (δ endotoxins, e.g., Cry toxin, Cyt toxin); a vegetative insecticidal protein (Vip), a nutrient (e.g., nitrogen, e.g., a leghemoglobin, a nitrogenase), a plant growth regulator (auxin, cytokinin, gibberellin, ethylene; growth inhibitor/retardant), an RNA (e.g., siRNA, dsRNA, miRNA, shRNA), a plantibody, a plant lipid, a plant fatty acid, a plant oil, a stylet sheath inhibitory protein (e.g., ficin, bromelain), a ribozyme, a bacteriocin, an antimicrobial peptide (e.g., oncocin), an aptamer, a nuclease, a zinc finger nuclease (ZFN), a Transcription Activator-Like Effector Nuclease (TALEN) and/or an engineered meganuclease.

93. The method of claim 89-92, wherein the at least one site on a host plant is on an above ground part of the host plant and/or on a below ground part of the host plant.

94. The method of any one of claims 89-91, wherein the symbiont or the symbiont forming inoculum is transplanted onto the host plant at least two times and/or onto at least two sites on the host plant.

95. The method of any one of claims 89-94, wherein the expression product is a transcription product or a translation product, or a modification thereof.

96. The method of any one of claims 89-95, wherein the product made using the expression product of the polynucleotide is a chemical, a protein (polypeptide/peptide) or a polynucleotide.

97. The method of any one of claims 89-96, wherein the modified host plant characteristic is increased tolerance/resistance to a disease causing organism (e.g., a fungus, a bacteria, a virus).

98. The method of any one of claims 89-97, wherein the modified host plant characteristic is increased (induced) expression of plant defense genes.

99. The method of any one of claims 89-98, wherein the modified host plant characteristic is increased insect tolerance/resistance.

100. The method of any one of claims 89-99, wherein the modified host plant characteristic is increased nematode tolerance/resistance.

101. The method of any one of claims 89-100 wherein the modified host plant characteristic is a modified morphology.

102. The method of claim 101, wherein the modified morphology comprises shortened internodes, increased lateral branching and/or increased flowering.

103. The method of any one of claims 89-102, wherein the modified host plant characteristic is the presence of a biomolecule, a bioactive molecule and/or a polypeptide involved in the biosynthesis of a bioactive molecule.

104. The method of claim 103, wherein the bioactive molecule is a pharmaceutical (e.g., a therapeutic protein, a therapeutic polynucleotide, a therapeutic chemical), a biostimulant, a biofungicide, a bioherbicide, an insecticidal protein/peptide, a trypsin modulating oostatic factor (TMOF); a Bacillus thuringiensis toxin, a vegetative insecticidal protein (Vip), a nutrient, a plant growth regulator, an RNA, a plantibody, a stylet sheath inhibitory protein, a ribozyme, a bacteriocin, an antimicrobial peptide, a plant lipid, a plant fatty acid, a plant oil, an aptamer, a nuclease, a zinc finger nuclease (ZFN), a Transcription Activator-Like Effector Nuclease (TALEN) and/or an engineered meganuclease.

105. The method of claim 103 or 104, wherein the bioactive molecule is jaburetox (peptide JBTX) an 5 endotoxin, a Cry toxin, a Cyt toxin, a leghemoglobin, a nitrogenase, ficin, bromelain a bacteriocin, nisin and/or oncocin).

106. The method of any one of claims 89-105, wherein the modified host plant characteristic is the presence of a bioactive molecule (e.g., biocidal molecule) in the host plant and increased resistance/tolerance to a plant pathogen.

107. The method of 106, wherein the bioactive molecule is a bacteriocin or an antimicrobial peptide and the plant pathogen is a bacterium.

108. The method of claim 107, wherein the bacteriocin or antimicrobial peptide is oncocin and/or nisin.

109. The method of any one of claims 89-105, wherein the modified host plant characteristic is the presence of an insecticidal protein (e.g., a bioinsecticide) and increased insect tolerance or resistance.

110. The method of claim 109, wherein the insecticidal protein is jaburetox, trypsin modulating oostatic factor (TMOF), a Bacillus thuringiensis toxin (e.g., δ endotoxins, e.g., Cry toxin, Cyt toxin); a stylet sheath inhibitory protein (e.g., ficin, bromelain), and/or a vegetative insecticidal protein (Vip).

111. The method of any one of claims 89-104, wherein the modified host plant characteristic is the presence of or increased or decreased production of a plant growth regulator (e.g., auxin, cytokinin, gibberellin, ethylene; a growth inhibitor/retardant) and modified growth.

112. The method of any one of claims 89-104, wherein the modified host plant characteristic is the presence of or increased production of an RNA and increased/decreased production of a polynucleotide, a peptide or a polypeptide.

113. The method of claim 112, wherein the RNA is a siRNA, a dsRNA, a miRNA, or a shRNA, optionally dvsnf7, ccomt, dCS, asn1, phL, RI, PGAS, and/or ppo5.

114. A host plant having a modified characteristic produced by the method of any one of claims 89 to 113.

115. A method of producing a biomolecule and/or bioactive molecule, comprising
providing the symbiont of any one of claims 26-42, wherein the polynucleotide of interest encodes a biomolecule and/or bioactive molecule and collecting the biomolecule and/or bioactive molecule produced in the symbiont; and/or
providing the host plant of any one of claims 43-47, wherein the polynucleotide of interest encodes a biomolecule and/or bioactive molecule and collecting the biomolecule and/or bioactive molecule produced in the symbiont and host plant.

116. A method of delivering a compound of interest to a host plant, comprising
transplanting the symbiont forming inoculum of any one of claims 1-25, 86, or 87, or the cell of claim 88 or the symbiont of any one of claims 26-42 onto at least one site on a host plant; and
culturing the symbiont forming inoculum or symbiont at the at least one site on the host plant to form a symbiont on the host plant at the at least one site, wherein the polynucleotide of interest is expressed in the symbiont and an expression product of the polynucleotide of interest and/or a product made using the expression product of the polynucleotide of interest is transported into the host plant, thereby delivering the compound of interest to a plant.

117. A method of producing a plant comprising a modified characteristic without modifying the plant's genotype, comprising:
transplanting the symbiont forming inoculum of any one of claims 1-25, 86, or 87, or the cell of claim 88, or the symbiont of any one of claims 26-42 onto at least one site on a host plant; and
culturing the symbiont forming inoculum or symbiont at the at least one site on the host plant to form a symbiont on the host plant at the at least one site, wherein the polynucleotide of interest is expressed in the symbiont and an expression product of the polynucleotide of interest and/or a product made using the expression product of the polynucleotide of interest is transported into the host plant, thereby producing the plant comprising a modified characteristic without a modified genotype.

118. A plant produced by the method of claim 117.
